(19) United States
(12) Patent Application Publication
(10) Pub. No.: US 2026/0185120 A1
(43) Pub. Date: Jul. 2, 2026

(54) MODIFIED MAMMALIAN CELLS FOR IMPROVED PRODUCTION

(71) Applicants: GENZYME CORPORATION, Cambridge, MA (US); THE REGENTS OF THE UNIVERSITY OF CALIFORNIA, Oakland, CA (US)

(72) Inventors: Qingliang LI, Lexington, MA (US); Yingchao NIE, Northborough, MA (US); Amit Kumar MATHUR, Lexington, MA (US); Karen Anne VINCENT, Carlisle, MA (US); Huimin NA, Belmont, MA (US); Madhuresh SUMIT, Marlborough, MA (US); Eunil IM, La Jolla, CA (US); Saratram GOPALAKRISHNAN, Athens, GA (US); Leo A. DWORKIN, San Diego, CA (US); Karen Julie la Cour KAROTTKI, Virum (DK); Nathan E. LEWIS, Athens, GA (US)

(21) Appl. No.: 19/366,807

(22) Filed: Oct. 23, 2025

Related U.S. Application Data
(60) Provisional application No. 63/711,825, filed on Oct. 25, 2024.
(60) Provisional application No. 63/711,808, filed on Oct. 25, 2024.
(60) Provisional application No. 63/879,099, filed on Sep. 10, 2025.

(51) Int. Cl. C12N 15/86

(52) U.S. Cl. CPC C12N 15/86; C12N 2710/10044; C12N 2750/14143; C12N 2750/14152

(57) ABSTRACT

Provided herein are engineered or modified cell lines in which the expression of at least one of ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, PARD6G-AS1, ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1 is altered as compared to a control cell line. Also provided are methods of producing recombinant AAV particles in said engineered or modified cell lines.

### CROSS-REFERENCE TO RELATED APPLICATIONS

This application claims priority to U.S. Provisional Patent Application Ser. No. 63/711,825, filed Oct. 25, 2024, 63/879,099, filed Sep. 10, 2025, and 63/711,808, filed Oct. 25, 2024, the contents of which are hereby incorporated by reference in their entireties.

### SEQUENCE LISTING

The instant application contains a Sequence Listing which has been submitted electronically in XML format and is hereby incorporated by reference in its entirety. Said XML file, created on Feb. 3, 2026, is named 770167_SA9-903_ST26.xml and is 9,845 bytes in size.

### BACKGROUND

Adeno-associated virus (AAV) is a DNA parvovirus that infects humans and various other animal species. AAV contains a single-stranded DNA genome encoding sets of replication (Rep) and capsid (Cap) proteins. Flanking the Rep and Cap open reading frames at the 5′ and 3′ ends are inverted terminal repeat sequences (ITRs), which act as origins of nucleic acid replication and as packaging signals for the virus. Recombinant AAV (rAAV)-based vectors are one of the most promising vehicles for human gene therapy. rAAV vectors are under consideration for a wide variety of gene therapy applications. In particular, rAAV vectors can deliver therapeutic genes to dividing and nondividing cells, and these genes can persist for extended periods without integrating into the genome of the target cells.

Although rAAV manufacturing has improved over the years, several issues remain. Development of rAAV-based therapies at the preclinical stage require large amounts of rAAV vectors to complete studies such as toxicology, biodistribution, and expression studies prior to clinical studies in human patients. However, current rAAV production results in lower than desirable titer yields. Therefore, there remains a need for more efficient rAAV production with higher viral titer for preclinical and clinical applications.

### SUMMARY OF THE INVENTION

In one aspect, provided herein is a engineered cell line in which the expression and/or activity of at least one of ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, PARD6G-AS1, RP11-268J15.6, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2 is altered as compared to a control cell line.

In certain embodiments, the expression of at least one of the transcription factor genes listed in Tables 1-4 is altered as compared to a control cell line. In certain embodiments, the expression of at least one of the transcription factor target genes listed in Tables 1-4 is altered as compared to a control cell line.

In certain embodiments, the expression of at least one gene is reduced and/or expression of at least one gene is increased, as compared to the control cell line.

In certain embodiments, the cell line is modified by a modulator that increases or decreases the expression and/or activity of at least one gene.

In certain embodiments, the cell line comprises a genetic modification.

In certain embodiments, the modulator is a nuclease. In certain embodiments, the nuclease is selected from the group consisting of zinc finger nuclease (ZFN), a meganuclease, a transcription activator-like effector nuclease (TALEN), and a clustered regularly interspaced short palindromic repeats (CRISPR) system.

In certain embodiments, the modulator is a double-stranded RNA (dsRNA), a small interfering RNA (siRNA), a small hairpin RNA (shRNA), a microRNA (miRNA), or an antisense RNA oligonucleotide (ASO).

In certain embodiments, the modulator comprises a template for homologous arm mediated recombination.

In certain embodiments, the modulator comprises an inhibitor of at least one gene selected from the group consisting of: KAT5, DBH, ITGAM, HHEX, HDAC5, SS18, FGFR3, RIGI/DDX58, HOXA5, FOXL2, ITGA2B, TNFAIP3, THEM6, HSP90B1, EHHADH, POLN, CHD1, NFKBIA, RELB, RXRA, CDKN1A, NFKB1 and ISYNA1. In certain embodiments, the inhibitor comprises a synthetic small molecule inhibitor.

In certain embodiments, the modulator comprises an agonist of at least one gene selected from the group consisting of: GATA1, LIF, ASNS, RPA1, TCF7, ADM2, NFKB2, BCL2, NME1, NR3C1, NGEF, GJB3, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1. In certain embodiments, the agonist comprises a synthetic small molecule agonist.

In certain embodiments, the cell line is a mammalian cell line or an insect cell line. In certain embodiments, the mammalian cell line is a CHO cell line, Vero cell line, HeLa cell line, MDCK cell line, BHK cell line, A549 cell line, amniocyte cell line, or HEK293 cell line. In certain embodiments, the mammalian cell line is a HeLa cell line or a human embryonic kidney (HEK) 293 cell line. In certain embodiments, the insect cell line is an Sf9 cell line or a Hi5 cell line.

In certain embodiments, the cell line is a modified or engineered host cell line for production of viral particles. In certain embodiments, the cell line is a producer cell line. In certain embodiments, the producer cell line further comprises stably integrated nucleic acid sequences encoding for AAV rep and/or cap, AAV ITR, and a gene of interest.

In certain embodiments, the producer cell line further comprises AAV helper function encoding sequences of an AAV helper virus. In certain embodiments, the AAV helper virus is an adenovirus. In certain embodiments, the adenovirus is Ad5 or Ad2. In certain embodiments, the AAV helper virus is a herpes simplex virus (HSV).

In certain embodiments, the AAV helper function encoding sequences comprise at least one of: E1a, E1b, E2A, L4, E4, and VA.

In certain embodiments, the titer of AAV produced from the engineered or modified cell line is at least about 1.5-fold higher compared to the titer of AAV produced from a cell line in which the expression of the gene is not altered.

In another aspect, provided herein is a method for identifying a gene associated with increasing the production of recombinant AAV (rAAV) particles from a cell, wherein the method comprises culturing the cell in the presence of a modulator that alters the expression or activity of at least one gene selected from the group consisting of: ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, and PARD6G-AS1, a transcription factor gene listed in Tables 1-4, and a transcription factor targeted gene listed in Tables 1-4, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2.

In certain embodiments, the method further comprises determining the titer of rAAV particles expressed from the cell and comparing the titer to a reference rAAV titer, wherein the reference rAAV titer is that of rAAV particles expressed from a cell that has not been cultured in the presence of the modulator but is otherwise identical.

In certain embodiments, if the titer of rAAV is increased compared to the reference rAAV titer, the gene is identified as a target for knock-down or knock-out in the cell. In certain embodiments, if the titer of rAAV particles is decreased compared to the reference rAAV titer, the gene is identified a target for over-expression in the cell.

In an aspect, provided herein is a method of modulating gene expression in a cell line for increasing the production of rAAV particles from a cell, wherein the method comprises culturing the cell in the presence of a modulator that alters the expression or activity of at least one gene selected from the group consisting of: ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, and PARD6G-AS1, a transcription factor gene listed in Tables 1-4, and a transcription factor targeted gene listed in Tables 1-4, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2.

In certain embodiments, the modulator is DMSO, DMF, NMP, or dihydrolevoglucosenone (Cyrene). In certain embodiments, the concentration of DMSO is at least about 0.5%.

In another aspect, provided herein is a method for increasing the production of recombinant AAV particles, wherein the method comprises culturing a cell in the presence of a modulator, wherein the expression or activity of at least one of the following genes: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3 is reduced as compared to a control cell line, and/or in which the expression of at least one of ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1 is increased as compared to a control cell line, under conditions that allow for production and/or secretion of the recombinant viral particles.

In certain embodiments, the method increases recombinant viral titer by at least 1.5-fold compared to a method comprising a control cell.

In certain embodiments, the modulator comprises a synthetic small molecule inhibitor of at least one of: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3.

In certain embodiments, the modulator comprises a synthetic small molecule agonist of at least one gene selected from the group consisting of: ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1.

In certain embodiments, the modulator is DMSO, DMF, NMP, or dihydrolevoglucosenone (Cyrene). In certain embodiments, the concentration of DMSO is at least about 0.5%. In certain embodiments, the modulator is an inhibitor or agonist of a pathway regulated by any one of the genes as disclosed in the present application.

In certain embodiments, the modulator is a nuclease. In certain embodiments, the nuclease is selected from the group consisting of zinc finger nuclease (ZFN), a meganuclease, a transcription activator-like effector nuclease (TALEN), a clustered regularly interspaced short palindromic repeats (CRISPR) system.

In certain embodiments, the modulator is a double stranded RNA (dsRNA), a small interfering RNA (siRNA), a small hairpin RNA (shRNA), a microRNA (miRNA), or an antisense RNA oligonucleotide (ASO).

In certain embodiments, the modulator comprises a template for homologous arm-mediated recombination.

In certain embodiments, the cell line is a mammalian cell line or an insect cell line.

In certain embodiments, the mammalian cell line is a CHO cell line, Vero cell line, HeLa cell line, MDCK cell line, BHK cell line, A549 cell line, amniocyte cell line, or human embryonic kidney (HEK) 293 cell line. In certain embodiments, the mammalian cell line is a HeLa cell line or a HEK293 cell line.

In certain embodiments, the insect cell line is an Sf9 cell line or a Hi5 cell line.

In an aspect, provided herein is an engineered or modified cell line produced by a method comprising knocking down the expression or activity of at least one of the genes selected from the group consisting of: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3.

In certain embodiments, the knockdown is carried out by using at least one of: homology directed recombination, RNA based knock-down, a CRISPR-Cas enzymatic method.

In yet another aspect, provided herein is an engineered or modified cell produced by a method comprising increasing the expression or activity of at least one of the genes selected from the group consisting of: ASNS, BCL2, CDKN1A, COL20A1, FAM46C, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2.

In certain embodiments, increasing the expression or activity is carried out by transient delivery or stable integration of a target expression modulation construct into the host cell line using at least one of: random integration, targeted integration (e.g., targeted integration at a safe harbor locus), homology directed recombination, a transposase-based recombination, a lentiviral based integration, an integrase mediated integration or a CRISPR-Cas activation method, optionally wherein the Cas comprises a dCas, such as dCas9.

In certain embodiments, the cell line further comprises stably integrated nucleic acid sequences encoding AAV Rep and/or cap, AAV ITR, and a gene of interest and optionally AAV helper function encoding sequences of an AAV helper virus.

In certain embodiments, the cell line further comprises transiently transfected nucleic acid sequences encoding for AAV rep and/or cap, AAV ITR, and a gene of interest and optionally AAV helper function encoding sequences of an AAV helper virus.

In certain embodiments, the AAV helper virus is an adenovirus. In certain embodiments, the adenovirus is Ad5 or Ad2. In certain embodiments, the AAV helper virus is a herpes simplex virus (HSV).

In certain embodiments, the AAV helper genes comprise at least one of: E1a, E1b, E2A, L4, E4, and VA.

In certain embodiments, provided herein is a recombinant viral particle obtained from the engineered or modified cell line as provided herein. In certain embodiments, the recombinant viral particle is a recombinant adeno-associated virus (rAAV) particle.

In an aspect, provided herein is a method for producing a recombinant adeno-associated virus (rAAV) particle, the method comprising: transiently transfecting the host cell line with one or more plasmids comprising: (i) nucleic acids encoding AAV rep and/or AAV cap, (ii) nucleic acids encoding AAV ITRs, (iii) nucleic acid encoding a gene of interest.

In certain embodiments, the method further comprises transfecting the host cell line with a plasmid comprising AAV helper function encoding sequences of an AAV helper virus. In certain embodiments, the AAV helper virus is an adenovirus. In some embodiments, the adenovirus is Ad5 or Ad2. In certain embodiments, the AAV helper virus is a herpes simplex virus (HSV).

In another aspect, provided herein is a method for producing a recombinant adeno-associated virus (rAAV) particle, the method comprising: stably transfecting the producer cell line as provided herein with: (i) nucleic acids encoding AAV Rep and/or AAV cap, (ii) nucleic acids encoding AAV ITRs, and (iii) a nucleic acid encoding a gene of interest.

In certain embodiments, the method further comprises transfecting the producer cell line with a plasmid comprising AAV helper function encoding sequences of an AAV helper virus. In certain embodiments, the AAV helper virus is an adenovirus. In certain embodiments, the adenovirus is Ad5 or Ad2. In certain embodiments, the AAV helper virus is a herpes simplex virus (HSV).

In certain embodiments, the rAAV comprises an AAV serotype selected from the group consisting of: AAV1, AAV2, AAV3, AAV4, AAV5, AAV6, AAV7, AAV8, AAVrh8, AAVrh8R, AAV9, AAV10, AAVrh10, AAV11, AAV12, AAV13, AAV DJ, a goat AAV, a bovine AAV, or a mouse AAV, AAV2/207m8, or AAV LK03, or hybrids, or variants or chimeras thereof.

In one aspect, provided herein is an engineered cell line in which the expression of glyceradehyde-3-phosphate dehydrogenase (GAPDH) is downregulated compared to a control cell line, wherein the cell line is a host cell line for the production of viral particles. In certain embodiments, the engineered cell line is an engineered human embryonic kidney (HEK) 293 cell line and the control cell line is a HEK293 cell line.

In one aspect, provided herein is an engineered human embryonic kidney (HEK) 293 cell line in which the expression of glyceradehyde-3-phosphate dehydrogenase (GAPDH) is downregulated compared to a control HEK293 cell line, wherein the HEK293 cell line is a host HEK293 cell line for the production of viral particles.

In certain embodiments, the GAPDH is downregulated by a modulator that decreases the expression and/or activity of the GAPDH. In certain embodiments, the cell line comprises a genetic modification. In certain embodiments, the modulator is a nuclease. In certain embodiments, the nuclease is selected from the group consisting of a zinc finger nuclease (ZFN), a meganuclease, a transcription activator-like effector nuclease (TALEN), and a clustered regularly interspaced short palindromic repeats (CRISPR) system. In certain embodiments, the modulator is a double stranded RNA (dsRNA), a small interfering RNA (siRNA), a small hairpin RNA (shRNA), a microRNA (miRNA), or an antisense RNA oligonucleotide (ASO). In certain embodiments, the modulator comprises a template for homologous arm-mediated recombination. In certain embodiments, the modulator comprises an inhibitor of GAPDH. In certain embodiments, the inhibitor comprises a synthetic small molecule inhibitor.

In one aspect, the HEK293 cell line is a producer HEK293 cell line.

In certain embodiments, the producer cell line further comprises stably integrated nucleic acid sequences encoding for AAV rep and/or cap, AAV ITR, and a gene of interest. In certain embodiments, the producer cell line further comprises AAV helper function encoding sequences of an AAV helper virus. In certain embodiments, the AAV helper virus is an adenovirus. In certain embodiments, the adenovirus is Ad5 or Ad2. In certain embodiments, the AAV helper function encoding sequences comprise at least one of: E1a, E1b, E2A, L4, E4, and VA. In certain embodiments, the titer of rAAV produced from the engineered cell line is at least about 1.5-fold higher compared to the titer of rAAV produced from a cell line in which the expression of the gene is not altered.

In certain aspects, the disclosure here provides a method of downregulating gene expression of glyceraldehyde-3-phosphate dehydrogenase (GAPDH) for increasing the production of rAAV particles from a cell, wherein the method comprises culturing the cell in the presence of a modulator that downregulates the expression or activity of GAPDH.

In certain embodiments, the modulator is dimethylsulfoxide (DMSO), dimethylformamide (DMF), (N-methylpyrrolidone (NMP), or dihydrolevoglucosenone (Cyrene). In certain embodiments, the concentration of DMSO is at least about 0.5%.

In certain aspects, the disclosure herein provides a method for increasing the production of rAAV particles, wherein the method comprises culturing a HEK293 cell in the presence of a modulator, wherein the expression or activity of glyceraldehyde-3-phosphate dehydrogenase (GAPDH) is decreased as compared to a control HEK293 cell line, under conditions that allow for production and/or secretion of the recombinant viral particles.

In certain embodiments, the method increases recombinant viral titer by at least 1.5-fold compared to a method comprising a control HEK293 cell. In certain embodiments, the modulator comprises a synthetic small molecule inhibitor of GAPDH. In some embodiments, the modulator is dimethylsulfoxide (DMSO), dimethylformamide (DMF), (N-methylpyrrolidone (NMP), dihydrolevoglucosenone (Cyrene), DC-5163, 1,2,3,4,6-penta-O-galloyl-β-D-glucopyranose (PGG), or 4-octyl itaconate (4-OI). In certain embodiments, the concentration of DMSO is at least about 0.5%. In certain embodiments, the modulator is an inhibitor of a pathway regulated by GAPDH. In some embodiments, the modulator is a nuclease. In certain embodiments, the nuclease is selected from the group consisting of a zinc finger nuclease (ZFN), a meganuclease, a transcription activator-like effector nuclease (TALEN), and a clustered regularly interspaced short palindromic repeats (CRISPR) system.

In certain embodiments, the modulator is a double stranded RNA (dsRNA), a small interfering RNA (siRNA), a small hairpin RNA (shRNA), a microRNA (miRNA), or an antisense RNA oligonucleotide (ASO). In certain embodiments, the modulator comprises a template for homologous arm-mediated recombination.

In certain aspects, the disclosure herein provides an engineered HEK293 host cell line produced by a method comprising knocking down the expression or activity of glyceraldehyde-3-phosphate dehydrogenase (GAPDH), wherein the engineered HEK293 cell line is for the production of viral particles.

In certain embodiments, the engineered HEK293 host cell line produced by the method of described herein, wherein the knockdown is carried out using at least one of: homology-directed recombination, RNA-based knock-down, and a CRISPR-Cas enzymatic method.

In certain embodiments, the engineered HEK293 host cell line produced by the method disclosed herein, further comprising stably integrated nucleic acid sequences encoding for AAV rep and/or cap, AAV ITR, and a gene of interest and optionally AAV helper function encoding sequences of an AAV helper virus.

In certain embodiments, the engineered HEK293 host cell line produced by the method disclosed herein, wherein the AAV helper virus is an adenovirus.

In certain embodiments, the engineered HEK293 host cell line produced by the method disclosed herein, wherein the adenovirus is Ad5 or Ad2.

In certain embodiments, the engineered HEK293 host cell line produced by the method of claim 100, wherein the AAV helper function encoding sequences comprise at least one of: E1a, E1b, E2A, L4, E4, and VA.

In certain embodiments, a recombinant viral particle obtained from the engineered HEK293 host cell line produced by the method described herein.

In certain embodiments, the recombinant viral particle is a recombinant adeno-associated virus (rAAV) particle.

In one aspect, disclosed herein a method for producing a recombinant adeno-associated virus (rAAV) particle, the method comprising: transiently transfecting the host HEK293 cell line disclosed herein with one or more plasmids comprising: (i) nucleic acids encoding AAV rep and/or AAV cap, (ii) nucleic acids encoding AAV ITRs, and (iii) nucleic acid encoding a gene of interest.

In certain embodiments, the method further comprising transfecting the host HEK293 cell line with a plasmid comprising AAV helper function encoding sequences of an AAV helper virus.

In certain embodiments, the AAV helper virus is an adenovirus. In certain embodiments, the adenovirus is Ad5 or Ad2.

In one aspect, as disclosed herein a method for producing a recombinant adeno-associated virus (rAAV) particle, the method comprising: stably transfecting the producer HEK293 cell line of claim 78 with: (i) nucleic acids encoding AAV rep and/or AAV cap, (ii) nucleic acids encoding AAV ITRs, and (iii) nucleic acid encoding a gene of interest.

In certain embodiments, the method further comprising transfecting the producer HEK293 cell line with a plasmid comprising AAV helper function encoding sequences of an AAV helper virus. In some embodiments, the AAV helper virus is an adenovirus. In certain embodiments, the adenovirus is Ad5 or Ad2. In certain embodiments, the rAAV comprises an AAV serotype selected from the group consisting of: AAV1, AAV2, AAV3, AAV4, AAV5, AAV6, AAV7, AAV8, AAVrh8, AAVrh8R, AAV9, AAV10, AAVrh10, AAV11, AAV12, AAV13, AAV DJ, a goat AAV, a bovine AAV, or a mouse AAV, AAV2/207m8, or AAV LK03, or hybrids, or variants or chimeras thereof.

### BRIEF DESCRIPTION OF THE DRAWINGS

FIGS. 1A-1F demonstrate that dimethyl sulfoxide (DMSO) enhances recombinant AAV (rAAV) production from a model producer cell line. FIG. 1A shows a schematic diagram of AAV producer cell line (PCL) and production. Triple play plasmids (TPP) harboring Rep, Cap and a Gene of Interest (GOI) were stably transfected into parental cells to generate the producer cell line. rAAV production is induced upon wtAd5 infection of PCL. FIG. 1B shows the effect of DMSO which increased AAV production from PCL1 in a dose-dependent manner up to 1%. DMSO concentrations higher than 1% (e.g., 1.5%, 2%, and 3%) resulted in no increase or lower levels of AAV production. AAV (FIG. 1B) and Ad5 (FIG. 1D) replication and production were analyzed from whole cell culture collected at 3 days post infection. AAV (FIG. 1B) and Ad5 (FIG. 1D) viral production was represented by DNase-resistant genome (DRG) copies. AAV (FIG. 1C) and Ad5 (FIG. 1E) viral replication was represented by total genome (TVG) copies. Relative rAAV volumetric productivity is shown as a bar graph and normalized to a non-DMSO control. 1% DMSO impact on AAV production was also assessed at typical infection density (X) as well as a 4-fold higher density (Y). 1% DMSO increased PCL1 production at both infection cell densities (FIG. 1F). P values were calculated using Prism 2-way ANOVA with n=3. Unless noted otherwise, X was the infection cell density for all the PCL culture infected at suspension condition for all studies, results shown in the subsequent figures.

FIGS. 2A-2G demonstrate that DMSO enhanced AAV cap protein expression and capsid production in PCL1. FIG. 2A is a schematic diagram of the experimental set up and sample collection. Two PCLs, PCL1 and PCL2 were infected in 250 mL shake flask with wtAd5 for rAAV production with or without 1% DMSO supplementation. DMSO was either added 3 hours prior infection (−3 h) or at the same time of infection (0 h), as shown in FIG. 2A. Samples were collected at 0 hpi, 24 hpi, 48 hpi and 72 hpi for cell viability % and Rep/Cap protein expression (western blot) detection. RNA-Seq samples were collected at 0 hpi, 24 hpi and 48 hpi from non-treated control flask and −3 h DMSO treated flask of both PCLs. rAAV genome and capsid titer were determined using qPCR and ELISA respectively using 72 hpi collected samples. FIG. 2B shows relative rAAV productivity. FIG. 2C shows AAV replication assessed by total vector genome copies via qPCR.

FIG. 2D shows AAV capsid production (VP) evaluated by ELISA. FIG. 2E shows AAV genome packaging percentage (DRG/TVG). FIG. 2F shows full capsid percentage. The full capsid percentages were estimated using DRG/VP. AAV Cap and Rep expression pattern of PCL1 (AAV2-SEAP) and PCL2 were determined using western blotting, and tubulin expression was used as loading control, as shown in FIG. 2G. All qPCR results were presented as bar graph in relative fold change normalized to non-DMSO control condition. Three independent experiments were conducted and average values were presented. P values were calculated using Prism 2-way ANOVA with n=3.

FIGS. 3A-3E demonstrate that DMSO did not significantly affect Ad5 production nor infection efficiency. FIG. 3A shows PCL1 and FIG. 3B shows PCL2 cell viability at 0 hpi, 24 hpi, 48 hpi and 72 hpi, assessed using the same experimental set up as described in FIG. 2A. In FIG. 3C relative Ad5 productivity is shown, as determined by qPCR, and presented as bar graph normalized to control condition of each PCL. FIG. 3D shows AdE1A protein expression of PCL1 and PCL2 at different time points as determined by western blotting. Tubulin expression was used as loading control. FIG. 3E shows the impact of DMSO on Ad5 infection. This was determined by assaying the infection efficiency of Ad5-GFP in PCL1 and PCL2. GFP positive cell percentages were captured using Celigo at 24 hpi. P values were calculated using Prism 2-way ANOVA with n=3.

FIGS. 4A-4D are provided to illustrate that DMSO treatment improved AAV productivity in Ambr®250 bioreactors with no adverse impact on vector quality. PCL1 cells were seeded into six Ambr®250 bioreactors pre-equilibrated with production medium at set pH, temperature and gas control. 1% DMSO was added to the three reactors at the time of cell seeding. wtAd5 infection was performed by adding wtAd5 at 3 h post cell inoculation. Cultures were harvested at 72 hpi for AAV productivity analysis and vector purification for quality understanding. FIG. 4A shows the relative AAV productivity from Ambr®250 bioreactors harvest. 1% DMSO treated samples were normalized to non-DMSO control. FIG. 1B shows VP ratios of purified vectors, using SDS-PAGE Coomassie blue staining. Equal numbers of purified vector genome copies of each sample were loaded onto SDS-PAGE based on purified vector titer. FIG. 4C shows capsid full percentage, as determined by analytical ultracentrifugation (AUC). FIG. 4D shows vector potency, which was evaluated with an enzyme activity assay specific for the transgene of PCL1. Cells were transduced with purified vectors from control or 1% DMSO treated condition at 1E3vg/cell and 1E4vg/cell. Culture supernatants were collected 6 days post transduction and assayed for the enzyme activity of the transgene. Relative activity from 1e3vg/cell and 1e4vg/cell transduction was shown as bar graph normalize to control condition. P values were calculated using Prism 2-way ANOVA with n=3.

FIG. 5A-5B show that DMSO enhanced rAAV productivity in a HEK293 transient transfection platform. HEK293 cells were transiently transfected to produce two rAAV vectors of different serotypes and transgenes along with mock or transfection only controls. 1% DMSO or culture medium was added at time of infection. Whole cells culture was collected at 3 days post-transfection (dpt) for AAV productivity analysis (FIG. 5A) and cell pellets were collected for KAT5 mRNA analysis (FIG. 5B). Relative rAAV productivity from 1% DMSO treatment is shown in FIG. 5A as bar graph normalized to each serotype non treated condition. Relative KAT5 mRNA expression from HEK293 cells treated with 1% DMSO is shown as a bar graph in FIG. 5B normalized to HEK293 non-transfection control without DMSO treatment. P values were calculated using Prism 2-way ANOVA with n=3.

FIGS. 6A-6C provide results of a screening study using siRNA-based knockdown, to elucidate possible mechanism of action of DMSO. To validate differentially expressed genes identified by RNA-Seq analysis, siRNA knockdown was performed as illustrated in FIG. 6A. AAV productivity upon target knockdown was assessed. Two siRNAs were used for each of the target selected. 20 μM of each siRNA were transfected into PCL1 cells using Lonza 4D nucleofector strip format. Cells were cultured for 2 days after resuspending and seeding into 24 well plates before infection with wtAd5 in 96 well plates with or without 1% DMSO. Whole infected culture was then collected at 48 hours post infection (4 days post siRNA transfection) for AAV productivity analysis via TaqMan qPCR. AAV productivity results are shown in FIG. 6B. Open circle represents AAV titers from control conditions and filled circle represents titers from 1% DMSO treated conditions. The siRNA results summary is shown in FIG. 6C to highlight one target: KAT5, which showed increased AAV productivity after siRNA knockdown but 1% DMSO did not lead to further increase of the productivity. siRNA RNA-Seq sample collection was carried out as described in Example 1 FIG. 2A. RNA-Seq analysis performed was described in Example 2.

FIGS. 7A-7E illustrate that the KAT5 knock down recapitulated the impact of DMSO on AAV Rep/Cap and Adenovirus E1A expression. siRNA transfection and wtAd5 infection were performed as described in FIG. 6A but using nucleocuvette for siRNA transfection instead of strip format. AAV productivity analysis results are shown in FIG. 7A. Consistent with siRNA screening results shown in FIG. 6B, siKAT5 increased AAV production to the similar level as seen in the 1% DMSO treatment. Combination of siKAT5 and DMSO did not show further AAV production improvement. Further analysis was carried out to determine the effect of siKAT5 treatment on AAV replication (FIG. 7B), Rep and Cap expression (FIG. 7C) and Ad5 E1A protein expression by western blot (FIG. 7D) and E1A mRNA expression (FIG. 7E). AAV replication and production were analyzed by qPCR, Rep/Cap and Ad5 E1A protein expression by Western blot, E1A mRNA analysis by RT-qPCR. All quantifications are presented as bar graphs using values from siN.C transfection and infection without DMSO as reference for normalization. Tubulin expression was used as loading control for western blot. P values were calculated using Prism 2-way ANOVA n=3.

FIGS. 8A-8C show the effect of DMSO treatment in additional PCLs. It was observed that DMSO increased AAV production partly via KAT5 in multiple PCLs. The impact of DMSO on additional PCL3 and PCL4 were tested along with PCL1 and PCL2, in deep well plates (FIG. 8A). 1% DMSO was added into the production medium at time of wtAd5 infection and AAV productivity was analyzed at 72 hpi. KAT5 knockdown using siKAT5 was evaluated in PCL1 to PCL6 (FIG. 8B), using experimental conditions similar to FIG. 6A. FIG. 8C shows KAT5 expression down-regulation upon DMSO treatment, as analyzed in PCL1 to PCL5 and parental cells, with or without wtAd5 infection. Producer cells were seeded into spin tubes and wtAd5 were added to one half of the total spin tubes at time of cell seeding for KAT5 expression analysis upon infection. 1% DMSO was added into half of the infected culture and half of the non-infection culture for evaluation of KAT5 expression following DMSO treatment with or without wtAd5 infection. Cell pellets were collected at 72 hpi and KAT5 expression was determined using RT-qPCR.

FIGS. 9A-9C demonstrate that DMSO supplementation affected cytokine production. In FIG. 9A differentially expressed cytokine gene transcripts from RNA-Seq analysis described in Example 2 are shown as a heatmap. FIG. 9B shows cytokine gene mRNA expression of PCL1 as determined by qPCR using a human cytokine array panel. Results were presented as relative log 2 fold change normalized to non-treated condition. Samples used for this analysis were collected 24 hpi. FIG. 9C shows the expression levels of secreted cytokine proteins determined using Luminex assay of cell culture supernatants. Results are shown as relative log 2 foldchange normalized to non-treated condition. Samples used for the analysis were collected at 24 hpi.

FIGS. 10A-10D provide the results of a study that shows DMSO and siKAT5 down-regulated CCND1 expression. To further investigate KAT5 targets identified in RNA-Seq analysis as described in Example 2, siKAT5 knockdown from PCL1 cells was performed as described in FIG. 7. These were analyzed for CCND1 and IL-6 mRNA expression using RT-qPCR. FIG. 10A shows CCND1 level was down-regulated upon DMSO treatment or siKAT5 knockdown alone. siKAT5 knockdown and DMSO together resulted in further reduction in CCND1 expression levels. As shown in FIG. 10B, IL-6 mRNA was only reduced in the DMSO treated condition. siKAT5 treatment did not lead to IL-6 mRNA expression down-regulation. CCND1 protein expression as determined by western blot (FIG. 10C) showed consistent pattern to that of CCND1 mRNA. Both siKAT5 or DMSO treatments, separately, reduced CCND1 protein expression and the two together had the lowest CCDN1 expression. CCND1 siRNA and IL-6 siRNA knockdown had no impact on AAV production under control or DMSO condition (FIG. 10D). siRNA transfection and AAV productivity assessment were performed as previously described in FIG. 6A. AAV productivities were normalized to that of siN.C no DMSO control and relative productivities were presented.

FIG. 11A-11C shows the acquisition and analysis pipeline of the RNA-Seq data to identify candidate regulators to enhance rAAV production. FIG. 11A shows PCL1 and PCL2 were cultured in medium with (treatment) and without (control) the 1% DMSO added. FIG. 11B shows that RNA-Seq data were collected and analyzed at 0, 24 and 48 hours from 36 samples. FIG. 11C shows the analysis workflow using a number of techniques, and identification of candidate target genes that could be associated with an increase in AAV titer.

FIG. 12A-12E shows the higher-level analysis of the RNA-Seq data. FIG. 12A illustrates significantly enriched biological processes (BP) and molecular functions (MF) Gene Ontology (GO) terms. The vertical dotted line represents a false discovery rate (FDR) corrected p-value cutoff of 0.05. GO terms with p-values<0.05 are considered significant. FIG. 12B-FIG. 12E is a series of heatmaps of genes that were differentially expressed for PCL1 and PCL2 cells cultured in control vs. DMSO-supplemented media over time for select GO terms.

FIG. 13A-13B illustrate the detailed immediate gene regulatory network of (A) KAT5 and (B) GATA1 and transcriptional support of their participation in the response to DMSO supplementation and accompanying increase in rAAV titer. FIG. 13A shows the immediate downstream regulatory network of KAT5, which is predicted to show inhibition upon DMSO treatment, based on the activation of downstream regulatory genes it is known to suppress or activate. Gene expression data supporting this and shown here comes from RNA-Seq at 0 hours post Ad5 infection. FIG. 13B shows the regulatory network immediately downstream and immediately upstream of GATA1, based on the expression of downstream targets at 0 hours post Ad5 infection.

FIGS. 14A-14B are heatmaps illustrating KAT5 and GATA1 downstream target gene expression as influenced by Ad5 infection over time. Log₂(Fold Change) expression was calculated at 0, 24, and 48 hours post Ad5 transfection for PCL1 and PCL2. Expression is ordered by time point for each clone and transcription factor targets are hierarchically clustered.

FIG. 15 illustrates an upregulation of CREB3L3 in PCLs cultured in media supplemented with DMSO when compared to cells cultured in control media. Fold-change comparison of DESeq2 RLE normalized counts between PCL1 in supplemented and control media (x-axis) and PCL1 and PCL2 in control media (y-axis) at time of infection (0 hpi). CREB3L3 is specifically indicated. Fold-Change>2 is indicated by grey dashed line in both comparisons. FDR p-adjusted values in either comparison are dark grey circles if <0.05, and dark grey diamonds if <0.05 with a fold-change>2 in both comparisons. Genes with adjusted p-value>0.05 not shown.

FIG. 16 depicts relative AAV titers following siRNA knockdown of select genes.

FIG. 17 depicts clustering of up-regulated genes in the AAV biosynthesis framework at 0 hours post infection (hpi). For each circle node, the radius represents the log 2 fold change.

FIG. 18 depicts clustering of up-regulated genes in the AAV biosynthesis framework at 3 hours post infection (hpi). For each circle node, the radius represents the log 2 fold change.

FIG. 19 is a graph illustrating rAAV productivity of HEK293 cells transfected with siRNA to GAPDH (siGAPDH) or control siRNA (siN.C). The cells were transfected with 100 μM, 200 μM, or 300 μM siRNA.

FIG. 20 is a graph illustrating rAAV productivity of HEK293 cells transfected with 100 μM siGAPDH or siN.C and AAV production transfection constructs with different Cap genes and different transgenes.

### DETAILED DESCRIPTION

Before the subject matter of the present disclosure is described, it is to be understood that the subject matter is not limited to particular methods and experimental conditions described as such methods and conditions can vary. The terminology used herein is for the purpose of describing particular embodiments only and is not intended to be limiting because the scope of the disclosure will be limited only by the appended claims.

Unless defined otherwise, all technical and scientific terms used herein have the same meaning as commonly understood by one of ordinary skill in the art to which this disclosure belongs.

The techniques and procedures described or referenced herein are described in Molecular Cloning: A Laboratory Manual (Sambrook et al., 4th ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6th ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011). All publications mentioned herein are incorporated herein by reference in their entirety.

### Definitions

As used herein, the term “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and non-viral vehicles for introducing the nucleic acid into a cell in vitro, ex vivo or in vivo, such as but not limited to a viral particle (e.g., an adeno-associated virus (AAV)). A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

As used herein, a “control” can refer to cells cultured in reference media without DMSO treatment. In addition, “control,” “control cell,” or “control cell line” when referring to the cells, refers to a cell that provides a reference point for measuring changes in genotype or phenotype of a modified or engineered cell. A control cell may comprise, for example: (a) a wild-type cell, i.e., of the same genotype as the starting material for the genetic alteration that resulted in the modified or engineered cell; (b) a cell of the same genotype as the modified or engineered cell but that has been transformed with a null construct (i.e., with a construct that has no known effect on a target gene of interest); or, (c) a cell genetically identical to the modified or engineered cell but that is not exposed to conditions or stimuli or further genetic modifications that would induce expression of altered genotype or phenotype. The control cell that is cultured in the reference media without DMSO treatment (untreated media) may be a wild-type (e.g., not engineered or modified) cell. In certain embodiments, the engineered or modified cell line has altered activity and/or expression of a target gene at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% compared to the control cell.

The terms “modified” or “engineered” as used herein describe a cell (e.g., a mammalian host cell) wherein expression of a gene is modified from its wild-type level of expression. The expression of a gene may be modified so that it is expressed at lower levels than wild-type levels. The expression of a gene may be modified so that it is expressed at higher levels than wild-type levels. The “modified” or “engineered” cell may be modified or engineered using at least one of a number of known genetic techniques, such as one that transiently or stably introduces an exogenous genetic cassette. The terms may also be used to describe a cell that has been exposed to an exogenous agent, e.g., a nucleic acid or oligonucleotide or a supplement such as DMSO that causes a modification (e.g., an increase or decrease in gene expression) in the cell. In some embodiments, the nucleic acid or oligonucleotide is a double stranded RNA (dsRNA), a small interfering RNA (siRNA), a small hairpin RNA (shRNA), a microRNA (miRNA), or an antisense RNA oligonucleotide (ASO).

In some embodiments, the terms “genetically modified” or “genetically engineered” can describe a cell in which the expression of at least one gene is reduced and/or the expression of at least one gene is increased through genetic modification of the cell's DNA. In some embodiments, the cell's DNA is modified by a modulator that increases or decreases the expression and/or activity of at least one gene. In some embodiments, the modulator is a nuclease. In some embodiments, the nuclease is selected from the group consisting of a zinc finger nuclease (ZFN), a meganuclease, a transcription activator-like effector nuclease (TALEN), and a clustered regularly interspaced short palindromic repeats (CRISPR) system. In some embodiments, the modulator comprises a template for homologous arm mediated recombination. In certain embodiments, the term “treatment cell line” as provided herein can be used interchangeably with a cell that is “modified” or “engineered”.

The term “plasmid” or “plasmid backbone” refers to an extrachromosomal circular DNA capable of autonomous replication in a given cell. A plasmid can include a selection gene in order to select or to identify a cell transfected therewith. Exemplary plasmids include but are not limited to those derived from pBR322, pUC, pUC19, pUC57, pJ241, or pJ247, pBluescript, pREP4, pCEP4, and pCI. Plasmids can also be engineered by standard molecular biology techniques (Sambrook et ah, Laboratory Manual, Cold Spring Harbor Laboratory Press, Cold Spring Harbor (1989), N.Y.).

As used herein, the term “gene” refers to the segment of a DNA molecule that codes for a polypeptide chain (e.g., the coding region). In some embodiments, a gene can include regions involved in producing the polypeptide chain that are positioned immediately preceding, following, and/or intervening the coding region (e.g., regulatory elements such as a promoter, enhancer, polyadenylation sequence, 5′-untranslated region, 3′-untranslated region, or intron).

As used herein, the term “regulatory element” refers to one or more nucleic acid molecules, such as promoters, enhancers, terminators, polyadenylation sequences, introns, and the like, that provide for the expression of a coding nucleic acid molecule in a cell.

As used herein, the term “promoter element” refers to a nucleic acid sequence that assists with controlling expression of a coding nucleic acid molecule. A promoter element can be located 5′ of the translation start site of a gene, or 3′ of the coding nucleic acid molecule. In some embodiments, a promoter useful for gene therapy can be derived from a native gene of a target protein. In some embodiments, a promoter useful for gene therapy can be specific for expression in a particular cell or tissue of the target organism (e.g., a liver-specific promoter or muscle-specific promoter). Non-limiting examples of well-characterized promoter elements include the CMV early promoter, CMV enhancer/chicken β-actin promoter (CGA), the 3-actin promoter, chicken β-actin (CBA) promoters, and the methyl CpG binding protein 2 (MeCP2) promoter. A promoter can be a constitutive promoter or an inducible promoter.

As used herein, the term the term “polynucleotide” or “nucleic acid” or “oligonucleotide” refers to a polymeric molecule of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-a-LNA having a 2′-amino functionalization) or hybrids thereof.

The terms “polypeptide” and “protein” are used interchangeably herein to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” refers to a protein which includes modifications, such as deletions, additions, and substitutions (generally conservative in nature), to the native nucleic acid molecule, as long as the protein maintains the desired activity. These modifications can be deliberate, as through site-directed mutagenesis, or can be accidental, such as through mutations of hosts which produce the proteins or errors due to PCR amplification.

As used herein, the term “naturally-occurring” as applied to an object refers to the fact that an object can be found in nature. For example, a polypeptide or polynucleotide, or components thereof such as amino acids or nucleotides, that is present in an organism (including viruses) that can be isolated from a source in nature and which has not been intentionally modified by man in the laboratory is naturally-occurring.

As used herein, the term “derivative” refers to a nucleic acid, peptide, or protein (e.g., an AAV capsid protein) or a variant or analog thereof comprising one or more mutations and/or chemical modifications as compared to a corresponding full-length wild-type nucleic acid, peptide or protein. Non-limiting examples of chemical modifications involving nucleic acids include, for example, modifications to the base moiety, sugar moiety, phosphate moiety, phosphate-sugar backbone, or a combination thereof. A nucleic acid molecule that encodes mutant gene constructs that can be useful with the plasmid system described herein can be identical to a wild type (i.e., unmutated) nucleic acid molecule or can be a different coding nucleic acid molecule, which nucleic acid molecule, as a result of the redundancy or degeneracy of the genetic code, encodes the same polypeptides as the wild type coding nucleic acid molecule. One of ordinary skill in the art will recognize that each codon in a nucleic acid molecule (except AUG, which is ordinarily the only codon for methionine, and TGG, which is ordinarily the only codon for tryptophan) can be modified to yield a functionally identical molecule. Accordingly, each variation of a nucleic acid which encodes a same polypeptide is implicit in each described sequence with respect to the expression product, but not with respect to actual gene therapy constructs.

One of ordinary skill in the art will recognize that individual substitutions, deletions or additions to a nucleic acid molecule that alters, adds or deletes a single amino acid or a small percentage of amino acids in the encoded nucleic acid molecule is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. Conservative amino acid substitutions providing functionally similar amino acids are well known in the art. Dependent on the functionality of the particular amino acid, e.g., catalytic, structural, or sterically important amino acids, different groupings of amino acid can be considered conservative substitutions for each other.

As used herein, the term “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

As used herein, the term “isolated” when referring to a molecule (e.g., nucleic acid or protein) or cell means the molecule or cell has been identified and separated and/or recovered from a component of its natural environment.

As used herein, the term “helper virus” for AAV refers to a virus that allows AAV to be replicated and packaged by a host cell. A number of helper viruses are known including adenoviruses, herpesviruses, baculovirus, and poxviruses such as vaccinia. Adenoviruses encompass a number of different subgroups. Numerous adenoviruses of human, non-human mammalian and avian origin are known and are readily available (e.g., from ATCC). Viruses of the herpes family include, for example, herpes simplex viruses (HSV), Epstein-Barr viruses (EBV), cytomegaloviruses (CMV), and pseudorabies viruses (PRV).

As used herein, the term “recombinant viral vector” refers to a recombinant polynucleotide vector comprising one or more heterologous nucleic acid molecules (i.e., nucleic acid molecule not of viral origin). In the case of recombinant AAV vectors, the recombinant nucleic acid molecule is flanked by at least one inverted terminal repeat sequence (ITR). In some embodiments, the recombinant nucleic acid molecule is flanked by two ITRs.

As used herein, the term “recombinant AAV vector (rAAV vector)” refers to a polynucleotide vector comprising one or more heterologous nucleic acid molecules (i.e., nucleic acid molecules not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV Rep and Cap gene products (i.e., AAV Rep and Cap proteins). When a rAAV vector is incorporated into a larger polynucleotide (e.g., in a chromosome or in another vector such as a plasmid used for cloning or transfection), then a rAAV vector can be referred to as a “pro-vector” which can be “rescued” by replication. An rAAV vector may be encapsidated in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”. “Recombinant AAV” (rAAV) and “AAV” are used interchangeably throughout the present disclosure.

As used herein, the term “rAAV virus” or “rAAV viral particle” refers to a viral particle composed of at least one AAV capsid protein and an encapsidated rAAV vector genome.

As used herein, the term “heterologous” means derived from a genotypically distinct entity from that of the rest of the entity to which it is compared or into which it is introduced or incorporated. As it relates to nucleic acid molecules, such as coding sequences and/or control sequences, the term denotes nucleic acid molecules that are not normally joined together and/or are not normally associated with a particular cell. A cellular sequence (e.g., a gene or portion thereof) that is incorporated into a viral vector is a heterologous nucleotide acid molecule with respect to the vector. Thus, a “heterologous” nucleic acid molecule can be a nucleic acid molecule from an organism other than AAV or which is synthetically derived.

As used herein, the term “operably-linked” refers to the association of two or more elements of a nucleic acid molecule that are physically linked so that the function of one of the elements is affected by another. For example, a regulatory DNA element is said to be “operably linked to” or “associated with” a DNA element that codes for an RNA or a polypeptide if the two elements are situated such that the regulatory DNA element affects expression of the coding DNA element (i.e., that the coding nucleic acid molecule or functional RNA is under the transcriptional control of the promoter). Coding nucleic acid molecules can be operably-linked to regulatory elements in sense or antisense orientation.

As used herein, the term “transgene” refers to a polynucleotide that is introduced into a cell and is capable of being transcribed into RNA and optionally, translated and/or expressed under appropriate conditions. A transgene can confer a desired property to a cell into which it was introduced, or otherwise leads to a desired therapeutic or diagnostic outcome. In another aspect, it can be transcribed into a molecule that mediates RNA interference, such as miRNA, siRNA, or shRNA. A transgene can be as few as a couple of nucleotides or at least about 50, 100, 150, 200, 250, 300, 350, 400, 500, 1,000, 2,000, 3,000, 4,000, 5,000, 6,000, 7,000, 7,500 nucleotides long or longer. A transgene can be for example, a viral genome. A transgene can be coding or non-coding nucleic acid molecule, or a combination thereof. A transgene can include one or more regulatory elements whereby transgene expression can be controlled.

As used herein, the terms “genome particles (gp)” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

As used herein, the term “vector genome (vg)” can refer to one or more polynucleotides comprising a set of the polynucleotide molecules of a vector, e.g., a viral vector. A vector genome can be encapsidated in a viral particle. Depending on the particular viral vector, a vector genome can comprise single-stranded DNA, double-stranded DNA, or single-stranded RNA, or double-stranded RNA. A vector genome can include endogenous nucleic acid molecules associated with a particular viral vector and/or any heterologous nucleic acid molecules inserted into a particular viral vector through recombinant techniques. For example, a recombinant AAV vector genome can include at least one ITR sequence flanking a promoter, a stuffer, a nucleic acid molecule of interest (e.g., an RNAi), and a polyadenylation sequence. A complete vector genome can include a complete set of the polynucleotide molecules of a vector. In some embodiments, the nucleic acid titer of a viral vector can be measured in terms of vector genome (vg/mL). Methods suitable for measuring this titer are known in the art (e.g., quantitative PCR).

As used herein, the term “inverted terminal repeat” or “ITR” sequence refers to relatively short sequences found at the termini of viral genomes which are in opposite orientation.

As used herein, the term “AAV inverted terminal repeat (ITR)” sequence refers to an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A, B, B′, C, C and D regions), allowing intra-strand base-pairing to occur within this portion of the ITR.

As used herein, the term “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

As used herein, the terms “subject”, “patient”, “individual”, and “animal” are used interchangeably and refer to mammals, including, without limitation, human and veterinary animals (e.g., cats, dogs, cows, horses, sheep, pigs, etc.) and experimental animal models. In certain embodiments, the individual or subject is a human.

As used herein, the term “treatment” refers to an approach for obtaining beneficial or desired clinical results. Beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment. The term “treat” is the verb form of “treatment”. “Treatment” can also refer to addition of a substance to a biological or non-biological sample in an attempt to cause a physical or chemical change in the substance.

As used herein, a “therapeutic” agent (e.g., a therapeutic polypeptide, nucleic acid, or transgene) is one that provides a beneficial or desired clinical result. As such, a therapeutic agent can be used in a treatment as described above.

Ranges can be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +10%, up to +5%, and up to +1% of a given value. For example, as used herein, the expression “about 100” includes 99 and 101 and all values in between (e.g., 99.1, 99.2, 99.3, 99.4, etc.) Alternatively, particularly with respect to biological systems or processes, the term can mean within an order of magnitude, or within 2-fold of a value. Where particular values are described in the application and claims, unless otherwise stated, the term “about” is implicit and in this context means within an acceptable error range for the particular value.

### Recombinant AAV Producer Cell Lines and Methods Thereof

In certain embodiments, the present disclosure provides compositions and methods for generating a producer cell line used for rAAV production. In certain embodiments, the compositions and methods comprise alteration of the expression of at least one of ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, and PARD6G-AS1 in a host cell line to generate an engineered or modified producer cell line. In certain embodiments, the expression of at least one of the transcription factor genes listed in Tables 1-4 is altered as compared to a control cell line. In certain embodiments, the expression of at least one of the transcription factor target genes is listed in Tables 1-4 is altered as compared to a control cell line.

In certain embodiments, provided herein is an engineered or modified host cell line produced by a method comprising knocking down the expression or activity of at least one of the genes selected from the group consisting of: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3. In certain embodiments, provided herein is an engineered or modified host cell line produced by a method comprising increasing the expression or activity of at least one of the genes selected from the group consisting of: ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, and PLEKHO2.

TABLE 1
TFs and all targets associated with increase
in viral titer as predicted by IPA for PCL1.

| | Transcription | |
|---|---|---|
| Transcription | Factor | |
| Factor | Target | Target Name |
| GATA1 | ALAS2 | 5′-aminolevulinate synthase 2 |
| | BCL2L1 | BCL2 like 1 |
| | GATA2 | GATA binding protein 2 |
| | GP9 | glycoprotein IX platelet |
| | IL13 | interleukin 13 |
| | ITGA2B | integrin subunit alpha 2b |
| | ITGAX | integrin subunit alpha X |
| | KIT | |
| | MBP | myelin basic protein |
| | PIM2 | Pim-2 proto-oncogene, serine/threonine |
| | | kinase |
| | PTGDR2 | prostaglandin D2 receptor 2 |
| | SKI | SKI proto-oncogene |
| | SLC19A1 | solute carrier family 19 member 1 |
| | SPTB | spectrin beta, erythrocytic |
| HOXA5 | BGLAP | bone gamma-carboxyglutamate protein |
| | CDKN1A | cyclin dependent kinase inhibitor 1A |
| | EGR1 | |
| | GADD45B | growth arrest and DNA damage inducible |
| | | beta |
| | LIF | LIF interleukin 6 family cytokine |
| | PTN | |
| | RUNX2 | RUNX family transcription factor 2 |
| | SAT1 | spermidine/spermine N1-acetyltransferase 1 |
| NGEF | BCL11B | BCL11 transcription factor B |
| | CCNJL | cyclin J like |
| | COL2A1 | collagen type II alpha 1 chain |
| | CXCR4 | C-X-C motif chemokine receptor 4 |
| | FGFR3 | fibroblast growth factor receptor 3 |
| | FLT4 | |
| | HEY1 | hes related family bHLH transcription factor |
| | | with YRPW (SEQ ID NO: 10) motif 1 |
| | IL12RB2 | interleukin 12 receptor subunit beta 2 |
| | LINC01140 | long intergenic non-protein coding RNA |
| | | 1140 |
| | MAFB | MAF bZIP transcription factor B |
| | SLC6A9 | solute carrier family 6 member 9 |
| | TAGLN | transgelin |
| | TERT | telomerase reverse transcriptase |
| | TNFSF10 | TNF superfamily member 10 |
| PELP1 | ADRB2 | adrenoceptor beta 2 |
| | ASNS | asparagine synthetase (glutamine- |
| | | hydrolyzing) |
| | BCL2L1 | BCL2 like 1 |
| | CCNG2 | cyclin G2 |
| | CDKN1A | cyclin dependent kinase inhibitor 1A |
| | CTSH | cathepsin H |
| | CXXC5 | CXXC finger protein 5 |
| | DGKA | diacylglycerol kinase alpha |
| | FGFR3 | fibroblast growth factor receptor 3 |
| | FOXQ1 | forkhead box Q1 |
| | LEP | leptin |
| | MAP1B | |
| | MRAS | muscle RAS oncogene homolog |
| | MRC1 | mannose receptor C-type 1 |
| | PRMT6 | protein arginine methyltransferase 6 |
| | SESN2 | sestrin 2 |
| | TAGLN | transgelin |
| SS18 | CABP7 | calcium binding protein 7 |
| | CACNA1G | calcium voltage-gated channel subunit |
| | | alphal G |
| | CBX4 | chromobox 4 |
| | GADD45B | growth arrest and DNA damage inducible |
| | | beta |
| | GCH1 | GTP cyclohydrolase 1 |
| | JAG2 | jagged canonical Notch ligand 2 |
| | NGFR | nerve growth factor receptor |
| | TENT5C | terminal nucleotidyltransferase 5C |
| RRAS2 | ADAMTS5 | ADAM metallopeptidase with |
| | | thrombospondin type 1 motif 5 |
| | CAV1 | caveolin 1 |
| | COL6A3 | |
| | FGB | fibrinogen beta chain |
| | FOSL1 | FOS like 1, AP-1 transcription factor subunit |
| | NID1 | |
| | PGF | placental growth factor |
| | TNFAIP3 | TNF alpha induced protein 3 |
| SORL1 | ABCA1 | ATP binding cassette subfamily A member 1 |
| | BMP2 | bone morphogenetic protein 2 |
| | CACHD1 | |
| | CAV1 | caveolin 1 |
| | CDH7 | |
| | COL5A2 | |
| | FGFR3 | fibroblast growth factor receptor 3 |
| | FGG | fibrinogen gamma chain |
| | KCND1 | potassium voltage-gated channel subfamily |
| | | D member 1 |
| | NID1 | |
| | NRP2 | neuropilin 2 |
| | PGF | placental growth factor |
| CHD1 | BMP2 | bone morphogenetic protein 2 |
| | CXCL2 | |
| | ICAM1 | intercellular adhesion molecule 1 |
| | IER3 | immediate early response 3 |
| | LIF | LIF interleukin 6 family cytokine |
| FOXL2 | CXCL2 | |
| | FAS | Fas cell surface death receptor |
| | ICAM1 | intercellular adhesion molecule 1 |
| | IER3 | immediate early response 3 |
| | LIF | LIF interleukin 6 family cytokine |
| | SPRY1 | sprouty RTK signaling antagonist 1 |
| | ZNF165 | zinc finger protein 165 |
| JAK1 | ICAM1 | intercellular adhesion molecule 1 |
| | JUNB | JunB proto-oncogene, AP-1 transcription |
| | | factor subunit |
| | LIF | LIF interleukin 6 family cytokine |
| | SMAD7 | |
| | TNS4 | |
| NPM1 | BMP2 | bone morphogenetic protein 2 |
| | COL6A2 | collagen type VI alpha 2 chain |
| | CXCL2 | |
| | DNAJB2 | DnaJ heat shock protein family (Hsp40) |
| | | member B2 |
| | FOXC2 | |
| | HILPDA | hypoxia inducible lipid droplet associated |
| | ICAM1 | intercellular adhesion molecule 1 |
| | JAG1 | jagged canonical Notch ligand 1 |
| | JUNB | JunB proto-oncogene, AP-1 transcription |
| | | factor subunit |
| | LIF | LIF interleukin 6 family cytokine |
| | MYO16 | |
| | NUAK1 | |
| | PGF | |
| | PMEPA1 | |
| | TGFBI | transforming growth factor beta induced |
| | TNFRSF25 | TNF receptor superfamily member 25 |
| | WNT5A | Wnt family member 5A |
| | ZC3H12A | zinc finger CCCH-type containing 12A |
| SORL1 | BMP2 | bone morphogenetic protein 2 |
| | EFNA1 | ephrin A1 |
| | EPHB2 | EPH receptor B2 |
| | FGF7 | fibroblast growth factor 7 |
| | FZD4 | frizzled class receptor 4 |
| | ICAM1 | intercellular adhesion molecule 1 |
| | NRP2 | neuropilin 2 |
| | PGF | |
| | RTN4R | |
| | SSTR1 | somatostatin receptor 1 |
| | WNT5A | Wnt family member 5A |

TABLE 2
TFs and all targets associated with increase
in viral titer as predicted by IPA for PCL2.

| | IPA TF | |
|---|---|---|
| IPA TF | target | Target Name |
| ANLN | ACER2 | alkaline ceramidase 2 |
| | AEN | apoptosis enhancing nuclease |
| | BTG2 | BTG anti-proliferation factor 2 |
| | CDH10 | cadherin 10 |
| | CDKN1A | cyclin dependent kinase inhibitor 1A |
| | CRACR2B | calcium release activated channel regulator |
| | | 2B |
| | DPYSL4 | dihydropyrimidinase like 4 |
| | GAL3ST4 | galactose-3-O-sulfotransferase 4 |
| | INKA2 | inka box actin regulator 2 |
| | MYH16 | myosin heavy chain 16 pseudogene |
| | PDE2A | phosphodiesterase 2A |
| | SESN2 | sestrin 2 |
| | SPATA18 | spermatogenesis associated 18 |
| | SULF2 | sulfatase 2 |
| | TIGAR | TP53 induced glycolysis regulatory |
| | | phosphatase |
| | TNFRSF10A | TNF receptor superfamily member 10a |
| | VWCE | von Willebrand factor C and EGF domains |
| BMP2 | BCL2L1 | BCL2 like 1 |
| | BGLAP | bone gamma-carboxyglutamate protein |
| | CDKN1A | cyclin dependent kinase inhibitor 1A |
| | COL1A2 | |
| | COL2A1 | collagen type II alpha 1 chain |
| | CTSK | cathepsin K |
| | DBH | dopamine beta-hydroxylase |
| | DLX2 | distal-less homeobox 2 |
| | F3 | coagulation factor III, tissue factor |
| | FGF2 | fibroblast growth factor 2 |
| | FST | follistatin |
| | GADD45B | growth arrest and DNA damage inducible |
| | | beta |
| | GREM1 | gremlin 1, DAN family BMP antagonist |
| | HAS2 | hyaluronan synthase 2 |
| | ID2 | inhibitor of DNA binding 2 |
| | IGFBP3 | insulin like growth factor binding protein 3 |
| | KLF10 | KLF transcription factor 10 |
| | KLF9 | KLF transcription factor 9 |
| | MRAS | muscle RAS oncogene homolog |
| | MSX1 | msh homeobox 1 |
| | MSX2 | msh homeobox 2 |
| | NOG | noggin |
| | PHOX2A | paired like homeobox 2A |
| | PLXNA2 | plexin A2 |
| | RUNX3 | RUNX family transcription factor 3 |
| | SMAD1 | SMAD family member 1 |
| | SMAD6 | SMAD family member 6 |
| | SMAD7 | SMAD family member 7 |
| | SOST | sclerostin |
| | SPARC | |
| | STAR | steroidogenic acute regulatory protein |
| | TAGLN | transgelin |
| | TIMP3 | |
| | TNFSF11 | TNF superfamily member 11 |
| | VDR | vitamin D receptor |
| | ZBTB2 | zinc finger and BTB domain containing 2 |
| NGEF | ART1 | ADP-ribosyltransferase 1 |
| | COL2A1 | collagen type II alpha 1 chain |
| | CXCR4 | C-X-C motif chemokine receptor 4 |
| | FGFR3 | fibroblast growth factor receptor 3 |
| | FLT4 | |
| | H2BC17 | H2B clustered histone 17 |
| | IL12RB2 | interleukin 12 receptor subunit beta 2 |
| | KIF5C | |
| | LILRA6 | leukocyte immunoglobulin like receptor A6 |
| | MAFB | MAF bZIP transcription factor B |
| | METTL7A | thiol methyltransferase 1A |
| | RYR1 | ryanodine receptor 1 |
| | SCARF1 | scavenger receptor class F member 1 |
| | SLC6A9 | solute carrier family 6 member 9 |
| | TAGLN | transgelin |
| | TAS2R4 | taste 2 receptor member 4 |
| | TERT | telomerase reverse transcriptase |
| | TNFSF10 | TNF superfamily member 10 |
| | UPK3B | uroplakin 3B |
| RASSF6 | BMI1 | BMI1 proto-oncogene, polycomb ring finger |
| | BTG2 | BTG anti-proliferation factor 2 |
| | CDKN1A | cyclin dependent kinase inhibitor 1A |
| | TP73 | tumor protein p73 |
| SS18 | CABP7 | calcium binding protein 7 |
| | CACNA1G | calcium voltage-gated channel subunit |
| | | alphal G |
| | CBX4 | chromobox 4 |
| | GADD45B | growth arrest and DNA damage inducible |
| | | beta |
| | GCH1 | GTP cyclohydrolase 1 |
| | MSX1 | msh homeobox 1 |
| | NFATC1 | nuclear factor of activated T cells 1 |
| | NGFR | nerve growth factor receptor |
| | PDGFB | platelet derived growth factor subunit B |
| | SLC34A2 | solute carrier family 34 member 2 |
| | TENT5C | terminal nucleotidyltransferase 5C |
| TFRC | ANKRD22 | ankyrin repeat domain 22 |
| | CDKN1A | cyclin dependent kinase inhibitor 1A |
| | FAM20C | FAM20C golgi associated secretory pathway |
| | | kinase |
| | ID2 | inhibitor of DNA binding 2 |
| | ITGAM | integrin subunit alpha M |
| | PLD4 | phospholipase D family member 4 |
| | PRTN3 | proteinase 3 |
| | SLC26A11 | solute carrier family 26 member 11 |
| | SLC2A14 | solute carrier family 2 member 14 |
| | SULF2 | sulfatase 2 |
| | TNFSF10 | TNF superfamily member 10 |
| KAT5 | ARC | activity regulated cytoskeleton associated |
| | | protein |
| | CCND1 | cyclin D1 |
| | IL6 | |
| | PTPRZ1 | |
| | VGF | VGF nerve growth factor inducible |
| BCOR | ADAMTS1 | ADAM metallopeptidase with |
| | | thrombospondin type 1 motif 1 |
| | ADM | adrenomedullin |
| | FOXQ1 | |
| | GAS1 | |
| CBX5 | CPA4 | carboxypeptidase A4 |
| | CYP1B1 | cytochrome P450 family 1 subfamily B |
| | | member 1 |
| | FGFBP1 | fibroblast growth factor binding protein 1 |
| | FOXA1 | forkhead box A1 |
| | FOXQ1 | |
| | IGFL2-AS1 | IGFL2 antisense RNA 1 |
| | KIT | |
| | MUC13 | mucin 13, cell surface associated |
| | OLR1 | oxidized low density lipoprotein receptor 1 |
| | RUNX2 | |
| | TIMP4 | TIMP metallopeptidase inhibitor 4 |
| | TM4SF1 | transmembrane 4 L six family member 1 |
| | TXNIP | thioredoxin interacting protein |
| CHD1 | BMP2 | bone morphogenetic protein 2 |
| | CXCL2 | C-X-C motif chemokine ligand 2 |
| | CXCL3 | C-X-C motif chemokine ligand 3 |
| | IER3 | immediate early response 3 |
| | IL6 | |
| | LIF | LIF interleukin 6 family cytokine |
| | PTGS2 | prostaglandin-endoperoxide synthase 2 |
| ECSIT | CXCL8 | C-X-C motif chemokine ligand 8 |
| | IER3 | immediate early response 3 |
| | IL6 | |
| | IRF7 | |
| | NFKBIA | NFKB inhibitor alpha |
| | PTGS2 | prostaglandin-endoperoxide synthase 2 |
| | TNFAIP3 | TNF alpha induced protein 3 |
| SCD | CEBPB | CCAAT enhancer binding protein beta |
| | CXCL3 | C-X-C motif chemokine ligand 3 |
| | CXCL8 | C-X-C motif chemokine ligand 8 |
| | HERPUD1 | homocysteine inducible ER protein with |
| | | ubiquitin like domain 1 |
| | HSPA5 | heat shock protein family A (Hsp70) member |
| | | 5 |
| THEM6 | CYP51A1 | |
| | HERPUD1 | homocysteine inducible ER protein with |
| | | ubiquitin like domain 1 |
| | HMGCS1 | 3-hydroxy-3-methylglutaryl-CoA synthase 1 |
| | HSP90B1 | heat shock protein 90 beta family member 1 |
| | HSPA5 | heat shock protein family A (Hsp70) member |
| | | 5 |
| | MSMO1 | methylsterol monooxygenase 1 |
| WWTR1 | BMP2 | bone morphogenetic protein 2 |
| | CXCL2 | C-X-C motif chemokine ligand 2 |
| | CXCL3 | C-X-C motif chemokine ligand 3 |
| | CXCL8 | C-X-C motif chemokine ligand 8 |
| | EFNA3 | |
| | FOXA1 | forkhead box A1 |
| | IL6 | |
| | IRF7 | |
| | IRX3 | iroquois homeobox 3 |
| | ITGB8 | integrin subunit beta 8 |
| | PLIN4 | |
| | THY1 | |
| | TNFSF9 | TNF superfamily member 9 |

TABLE 3
Additional TFs and targets associated with increase
in viral titer as predicted by IPA for PCL1.

| IPA | IPA TF | |
|---|---|---|
| TF | target | Target Name |
| ADM2 | IGFBP3 | insulin like growth factor binding protein 3 |
| | LEP | leptin |
| | MTUS1 | microtubule associated scaffold protein 1 |
| | TNFRSF9 | |
| BCL2 | BCL2L1 | BCL2 like 1 |
| | CCNE1 | cyclin E1 |
| | CDKN1A | cyclin dependent kinase inhibitor 1A |
| | NES | nestin |
| | PRDM1 | PR/SET domain 1 |
| CCND1 | AREG | amphiregulin |
| | CCNE1 | cyclin E1 |
| | CDCA2 | cell division cycle associated 2 |
| | CDK6 | cyclin dependent kinase 6 |
| | CDKN1A | cyclin dependent kinase inhibitor 1A |
| | CPED1 | cadherin like and PC-esterase domain |
| | | containing 1 |
| | FZD7 | frizzled class receptor 7 |
| | GNMT | glycine N-methyltransferase |
| | H2BC21 | H2B clustered histone 21 |
| | H4C8 | H4 clustered histone 8 |
| | IER3 | immediate early response 3 |
| | ITGB3 | integrin subunit beta 3 |
| | ITIH2 | inter-alpha-trypsin inhibitor heavy chain 2 |
| | LAMB2 | laminin subunit beta 2 |
| | MAP3K5 | mitogen-activated protein kinase kinase |
| | | kinase 5 |
| | MYBL1 | MYB proto-oncogene like 1 |
| | MYO7A | myosin VIIA |
| | MYT1 | myelin transcription factor 1 |
| | RGS2 | regulator of G protein signaling 2 |
| | SATB1 | |
| | SCG5 | secretogranin V |
| | SI | sucrase-isomaltase |
| | TM7SF2 | transmembrane 7 superfamily member 2 |
| | TMEM204 | transmembrane protein 204 |
| | TP53INP2 | tumor protein p53 inducible nuclear |
| | | protein 2 |
| | UAP1 | UDP-N-acetylglucosamine |
| | | pyrophosphorylase 1 |
| FGFR3 | BCL2L1 | BCL2 like 1 |
| | CDKN1A | cyclin dependent kinase inhibitor 1A |
| | CLDN5 | claudin 5 |
| | FRMD3 | FERM domain containing 3 |
| | ZNF589 | zinc finger protein 589 |
| GATA1 | ITGA2B | integrin subunit alpha 2b |
| HDAC5 | ACTA1 | actin alpha 1, skeletal muscle |
| | CDKN1A | cyclin dependent kinase inhibitor 1A |
| | CKM | creatine kinase, M-type |
| | DAPK1 | death associated protein kinase 1 |
| | NR4A1 | nuclear receptor subfamily 4 group |
| | | A member 1 |
| | TAGLN | transgelin |
| | TNFRSF1A | TNF receptor superfamily member 1A |
| | TUBB3 | tubulin beta 3 class III |
| HHEX | CCN1 | cellular communication network factor 1 |
| | CCN2 | cellular communication network factor 2 |
| | FGF2 | fibroblast growth factor 2 |
| | IL12A | interleukin 12A |
| | IL15 | interleukin 15 |
| | IL17F | interleukin 17F |
| | PPBP | pro-platelet basic protein |
| | SLC10A1 | solute carrier family 10 member 1 |
| | TNFSF10 | TNF superfamily member 10 |
| | TNFSF14 | TNF superfamily member 14 |
| HNF4A | TCF7 | transcription factor 7 |
| HOXA5 | LIF | LIF interleukin 6 family cytokine |
| LAS1L | CCNG2 | cyclin G2 |
| | CDKN1A | cyclin dependent kinase inhibitor 1A |
| | CHST6 | carbohydrate sulfotransferase 6 |
| | CPT1C | carnitine palmitoyltransferase 1C |
| | CRABP2 | cellular retinoic acid binding protein 2 |
| | DGKA | diacylglycerol kinase alpha |
| | SESN2 | sestrin 2 |
| | SUSD2 | sushi domain containing 2 |
| NGEF | BCL11B | BCL11 transcription factor B |
| | CCNJL | cyclin J like |
| | COL2A1 | collagen type II alpha 1 chain |
| | CXCR4 | C-X-C motif chemokine receptor 4 |
| | FGFR3 | fibroblast growth factor receptor 3 |
| | FLT4 | |
| | HEY1 | hes related family bHLH transcription |
| | | factor with YRPW (SEQ ID NO: 10) motif 1 |
| | IL12RB2 | interleukin 12 receptor subunit beta 2 |
| | LINC01140 | long intergenic non-protein coding |
| | | RNA 1140 |
| | MAFB | MAF bZIP transcription factor B |
| | SLC6A9 | solute carrier family 6 member 9 |
| | TAGLN | transgelin |
| | TERT | telomerase reverse transcriptase |
| | TNFSF10 | TNF superfamily member 10 |
| NME1 | CCN2 | cellular communication network factor 2 |
| | CDKN1A | cyclin dependent kinase inhibitor 1A |
| | FZD1 | frizzled class receptor 1 |
| | GEMIN5 | gem nuclear organelle associated |
| | | protein 5 |
| | MMP14 | matrix metallopeptidase 14 |
| | PTN | |
| NR3C1 | LIF | LIF interleukin 6 family cytokine |
| PELP1 | FGFR3 | fibroblast growth factor receptor 3 |
| PTEN | AKAP1 | A-kinase anchoring protein 1 |
| | AKR1C3 | aldo-keto reductase family 1 |
| | | member C3 |
| | ARID2 | AT-rich interaction domain 2 |
| | BCL3 | BCL3 transcription coactivator |
| | BPGM | bisphosphoglycerate mutase |
| | CBX4 | chromobox 4 |
| | CDKN1A | cyclin dependent kinase inhibitor 1A |
| | CXCL11 | C-X-C motif chemokine ligand 11 |
| | CXCR4 | C-X-C motif chemokine receptor 4 |
| | CYP27A1 | |
| | DUSP1 | dual specificity phosphatase 1 |
| | F3 | coagulation factor III, tissue factor |
| | FGFR3 | fibroblast growth factor receptor 3 |
| | GATA2 | GATA binding protein 2 |
| | GGT1 | gamma-glutamyltransferase 1 |
| | HCLS1 | hematopoietic cell-specific Lyn substrate 1 |
| | HCN2 | hyperpolarization activated cyclic |
| | | nucleotide gated potassium and sodium |
| | | channel 2 |
| | HGFAC | HGF activator |
| | IDI1 | isopentenyl-diphosphate delta isomerase 1 |
| | IFNGR2 | interferon gamma receptor 2 |
| | IGFBP3 | insulin like growth factor binding protein 3 |
| | IL4R | interleukin 4 receptor |
| | JUND | JunD proto-oncogene, AP-1 transcription |
| | | factor subunit |
| | NFIL3 | nuclear factor, interleukin 3 regulated |
| | NR3C1 | nuclear receptor subfamily 3 group C |
| | | member 1 |
| | PLAU | plasminogen activator, urokinase |
| | PRDM1 | PR/SET domain 1 |
| | SAT1 | spermidine/spermine N1-acetyltransferase 1 |
| | SH2B2 | SH2B adaptor protein 2 |
| | THBS1 | thrombospondin 1 |
| | TNFRSF10D | TNF receptor superfamily member 10d |
| | TNFRSF14 | TNF receptor superfamily member 14 |
| | TNFRSF9 | |
| | TNFSF10 | TNF superfamily member 10 |
| RXRA | TCF7 | transcription factor 7 |
| SMAD4 | ACVR1C | activin A receptor type 1C |
| | BGLAP | bone gamma-carboxyglutamate protein |
| | CCN2 | cellular communication network factor 2 |
| | CCNE1 | cyclin E1 |
| | CCNG2 | cyclin G2 |
| | CDK17 | cyclin dependent kinase 17 |
| | CDK6 | cyclin dependent kinase 6 |
| | CDKN1A | cyclin dependent kinase inhibitor 1A |
| | COL1A2 | |
| | CSF1 | colony stimulating factor 1 |
| | DAPK1 | death associated protein kinase 1 |
| | EPB41L5 | erythrocyte membrane protein band 4.1 |
| | | like 5 |
| | FST | follistatin |
| | FSTL3 | follistatin like 3 |
| | GADD45B | growth arrest and DNA damage inducible |
| | | beta |
| | GUCY2F | guanylate cyclase 2F, retinal |
| | HMCN1 | hemicentin 1 |
| | HMGA2 | high mobility group AT-hook 2 |
| | HMOX1 | heme oxygenase 1 |
| | ID2 | inhibitor of DNA binding 2 |
| | IER3 | immediate early response 3 |
| | ITGB7 | integrin subunit beta 7 |
| | JAG2 | jagged canonical Notch ligand 2 |
| | MUC4 | mucin 4, cell surface associated |
| | PILRA | paired immunoglobin like type 2 receptor |
| | | alpha |
| | PLAU | plasminogen activator, urokinase |
| | SERTAD1 | SERTA domain containing 1 |
| | SPI1 | Spi-1 proto-oncogene |
| | SSTR2 | somatostatin receptor 2 |
| | TERT | telomerase reverse transcriptase |
| | THBS1 | thrombospondin 1 |
| | TIMP3 | TIMP metallopeptidase inhibitor 3 |
| | TNFRSF10A | TNF receptor superfamily member 10a |
| SS18 | CABP7 | calcium binding protein 7 |
| | CACNA1G | calcium voltage-gated channel subunit |
| | | alpha1 G |
| | CBX4 | chromobox 4 |
| | GADD45B | growth arrest and DNA damage inducible |
| | | beta |
| | GCH1 | GTP cyclohydrolase 1 |
| | JAG2 | jagged canonical Notch ligand 2 |
| | NGFR | nerve growth factor receptor |
| | TENT5C | terminal nucleotidyltransferase 5C |
| TCF7 | AXIN2 | axin 2 |
| | BCL6 | BCL6 transcription repressor |
| | MMP14 | matrix metallopeptidase 14 |
| | NES | nestin |
| | PRDM1 | PR/SET domain 1 |
| | RORC | RAR related orphan receptor C |
| | SPI1 | Spi-1 proto-oncogene |
| | TCF7 | transcription factor 7 |
| TGM2 | BCL6 | BCL6 transcription repressor |
| | CCRL2 | C-C motif chemokine receptor like 2 |
| | CDK6 | cyclin dependent kinase 6 |
| | CFP | complement factor properdin |
| | CSRNP1 | cysteine and serine rich nuclear |
| | | protein 1 |
| | E2F5 | E2F transcription factor 5 |
| | ICAM3 | intercellular adhesion molecule 3 |
| | ITGAM | integrin subunit alpha M |
| | ITGAX | integrin subunit alpha X |
| | ITGB3 | integrin subunit beta 3 |
| | LGALS9 | galectin 9 |
| | MAFB | MAF bZIP transcription factor B |
| | NLRC4 | NLR family CARD domain containing 4 |
| | PLEKHO2 | pleckstrin homology domain containing O2 |
| | RFLNB | refilin B |
| | SEMA7A | semaphorin 7A (John Milton Hagen blood |
| | | group) |
| | SERPINB2 | serpin family B member 2 |
| | SIGLEC12 | sialic acid binding Ig like lectin 12 |
| | SLFN5 | schlafen family member 5 |
| | THBS1 | thrombospondin 1 |
| | UBA7 | ubiquitin like modifier activating |
| | | enzyme 7 |
| TP53 | LIF | LIF interleukin 6 family cytokine |
| TRPC1 | APAF1 | apoptotic peptidase activating factor 1 |
| | CDK6 | cyclin dependent kinase 6 |
| | CDKN1A | cyclin dependent kinase inhibitor 1A |
| | TRPC1 | transient receptor potential cation |
| | | channel subfamily C member 1 |
| IL6 | ABCA1 | ATP binding cassette subfamily A member 1 |
| | ADAMTS5 | ADAM metallopeptidase with thrombospondin |
| | | type 1 motif 5 |
| | CCND1 | cyclin D1 |
| | CYBB | |
| | FGA | fibrinogen alpha chain |
| | FGB | fibrinogen beta chain |
| | FGG | fibrinogen gamma chain |
| | FLI1 | |
| | GP1BA | |
| | IFIT1 | interferon induced protein with |
| | | tetratricopeptide repeats 1 |
| | IL1RL1 | |
| | ITGB3 | |
| | KIT | |
| | MAP2 | |
| | NES | nestin |
| | PCSK1 | |
| | PGF | placental growth factor |
| | PLEK | |
| | PPBP | pro-platelet basic protein |
| | RUNX2 | RUNX family transcription |
| | | factor 2 |
| | TERT | |
| | THBS1 | thrombospondin 1 |
| | TLR3 | |
| MAP2K1 | CCND1 | cyclin D1 |
| | ITGA2B | |
| PRKCA | CCND1 | cyclin D1 |
| | TRPC1 | |
| SALL4 | CCND1 | cyclin D1 |
| SORL1 | FGFR3 | fibroblast growth factor |
| | | receptor 3 |
| SP1 | CCND1 | cyclin D1 |
| STAT3 | CCND1 | cyclin D1 |
| SYVN1 | CCND1 | cyclin D1 |
| BCL2L1 | FAS | Fas cell surface death receptor |
| BRD4 | CCND1 | cyclin D1 |
| CHD1 | LIF | LIF interleukin 6 family cytokine |
| EGFR | CCND1 | cyclin D1 |
| ESR2 | CCND1 | cyclin D1 |
| FOXL2 | FAS | Fas cell surface death receptor |
| | LIF | LIF interleukin 6 family cytokine |
| HGF | CCND1 | cyclin D1 |
| JAK1 | LIF | LIF interleukin 6 family cytokine |
| JAK2 | CCND1 | cyclin D1 |
| NPM1 | LIF | LIF interleukin 6 family cytokine |
| RAF1 | CCND1 | cyclin D1 |
| | FAS | Fas cell surface death receptor |
| | LIF | LIF interleukin 6 family cytokine |
| SIX2 | FAS | Fas cell surface death receptor |
| STAT3 | CCND1 | cyclin D1 |
| | FAS | Fas cell surface death receptor |
| | LIF | LIF interleukin 6 family cytokine |
| SYVN1 | CCND1 | cyclin D1 |
| TGFA | CCND1 | cyclin D1 |
| TGM2 | C5AR1 | complement C5a receptor 1 |
| | CCND1 | cyclin D1 |
| | CCRL2 | |
| | DAPK2 | death associated protein kinase 2 |
| | E2F5 | E2F transcription factor 5 |
| | GCA | |
| | IFI35 | interferon induced protein 35 |
| | ITGB3 | |
| | MAFB | |
| | MDN1 | midasin AAA ATPase 1 |
| | SMPD1 | sphingomyelin phosphodiesterase 1 |
| | THBS1 | |

TABLE 4
Additional TFs and targets associated with increase
in viral titer as predicted by IPA for PCL2

| IPA | IPA TF | |
|---|---|---|
| TF | target | Target Name |
| APP | NFKB1 | nuclear factor kappa B subunit 1 |
| BCL2 | BCL2L1 | BCL2 like 1 |
| | CCNE1 | cyclin E1 |
| | CDKN1A | cyclin dependent kinase inhibitor 1A |
| | NES | nestin |
| | PRDM1 | PR/SET domain 1 |
| CCND1 | AR | androgen receptor |
| | AREG | amphiregulin |
| | CCNE1 | cyclin E1 |
| | CDK6 | cyclin dependent kinase 6 |
| | CDKN1A | cyclin dependent kinase inhibitor 1A |
| | CPED1 | cadherin like and PC-esterase domain |
| | | containing 1 |
| | E2F8 | E2F transcription factor 8 |
| | EPHA2 | EPH receptor A2 |
| | ERRFI1 | ERBB receptor feedback inhibitor 1 |
| | FZD7 | frizzled class receptor 7 |
| | H2BC21 | H2B clustered histone 21 |
| | H4C8 | H4 clustered histone 8 |
| | HOMER2 | homer scaffold protein 2 |
| | IER3 | immediate early response 3 |
| | ITGB3 | |
| | ITIH2 | inter-alpha-trypsin inhibitor heavy |
| | | chain 2 |
| | KDM6B | lysine demethylase 6B |
| | KRT4 | keratin 4 |
| | LAMB2 | laminin subunit beta 2 |
| | MAGI2 | membrane associated guanylate kinase, |
| | | WW and PDZ domain containing 2 |
| | MARCKS | |
| | MTFR2 | mitochondrial fission regulator 2 |
| | MYBL1 | MYB proto-oncogene like 1 |
| | MYO7A | myosin VIIA |
| | MYT1 | myelin transcription factor 1 |
| | RGS2 | regulator of G protein signaling 2 |
| | SCG5 | secretogranin V |
| | SI | sucrase-isomaltase |
| | TMEM204 | transmembrane protein 204 |
| | TP53INP2 | tumor protein p53 inducible nuclear |
| | | protein 2 |
| | UAP1 | UDP-N-acetylglucosamine |
| | | pyrophosphorylase 1 |
| CTNNB1 | LIF | LIF interleukin 6 family cytokine |
| | TCF7 | transcription factor 7 |
| F2 | TRPC1 | transient receptor potential cation |
| | | channel subfamily C member 1 |
| HDAC5 | ACTA1 | actin alpha 1, skeletal muscle |
| | CD27 | CD27 molecule |
| | CDKN1A | cyclin dependent kinase inhibitor 1A |
| | CKM | creatine kinase, M-type |
| | DAPK1 | death associated protein kinase 1 |
| | LMOD2 | leiomodin 2 |
| | MEG3 | |
| | NR4A1 | nuclear receptor subfamily 4 group A |
| | | member 1 |
| | SMAD7 | SMAD family member 7 |
| | TAGLN | transgelin |
| | TNFRSF1A | TNF receptor superfamily member 1A |
| | TUBB3 | tubulin beta 3 class III |
| HHEX | CCN1 | cellular communication network factor 1 |
| | CCN2 | cellular communication network factor 2 |
| | FGF2 | fibroblast growth factor 2 |
| | IL17F | interleukin 17F |
| | PPBP | |
| | SLC10A1 | solute carrier family 10 member 1 |
| | TNFSF10 | TNF superfamily member 10 |
| | TNFSF14 | TNF superfamily member 14 |
| HIF1A | ITGA2B | integrin subunit alpha 2b |
| HIVEP1 | NFKB1 | nuclear factor kappa B subunit 1 |
| IL13 | FGFR3 | fibroblast growth factor receptor 3 |
| LAS1L | BTG2 | BTG anti-proliferation factor 2 |
| | CCNG2 | cyclin G2 |
| | CDKN1A | cyclin dependent kinase inhibitor 1A |
| | CEMIP | cell migration inducing hyaluronidase 1 |
| | CHST6 | carbohydrate sulfotransferase 6 |
| | CPT1C | carnitine palmitoyltransferase 1C |
| | SESN2 | sestrin 2 |
| | SPACA6 | sperm acrosome associated 6 |
| | SUSD2 | sushi domain containing 2 |
| NGEF | ART1 | ADP-ribosyltransferase 1 |
| | COL2A1 | collagen type II alpha 1 chain |
| | CXCR4 | C-X-C motif chemokine receptor 4 |
| | FGFR3 | fibroblast growth factor receptor 3 |
| | FLT4 | |
| | H2BC17 | H2B clustered histone 17 |
| | IL12RB2 | interleukin 12 receptor subunit beta 2 |
| | KIF5C | |
| | LILRA6 | leukocyte immunoglobulin like receptor A6 |
| | MAFB | MAF bZIP transcription factor B |
| | METTL7A | thiol methyltransferase 1A |
| | RYR1 | ryanodine receptor 1 |
| | SCARF1 | scavenger receptor class F member 1 |
| | SLC6A9 | solute carrier family 6 member 9 |
| | TAGLN | transgelin |
| | TAS2R4 | taste 2 receptor member 4 |
| | TERT | telomerase reverse transcriptase |
| | TNFSF10 | TNF superfamily member 10 |
| | UPK3B | uroplakin 3B |
| NME1 | CCN2 | cellular communication network factor 2 |
| | CDKN1A | cyclin dependent kinase inhibitor 1A |
| | FZD1 | frizzled class receptor 1 |
| | GEMIN5 | gem nuclear organelle associated protein 5 |
| | METTL1 | methyltransferase 1, tRNA methylguanosine |
| | MMP14 | matrix metallopeptidase 14 |
| | PTN | |
| | SMAD7 | SMAD family member 7 |
| NR3C1 | LIF | LIF interleukin 6 family cytokine |
| | NFKB1 | nuclear factor kappa B subunit 1 |
| PAF1 | DDX58 | RNA sensor RIG-I |
| PRKCD | LIF | LIF interleukin 6 family cytokine |
| | TRPC1 | transient receptor potential cation |
| | | channel subfamily C member 1 |
| PTEN | ADM | adrenomedullin |
| | AKAP1 | A-kinase anchoring protein 1 |
| | AKR1C3 | aldo-keto reductase family 1 member C3 |
| | AR | androgen receptor |
| | BCL3 | BCL3 transcription coactivator |
| | BMI1 | BMI1 proto-oncogene, polycomb ring finger |
| | CBX4 | chromobox 4 |
| | CDKN1A | cyclin dependent kinase inhibitor 1A |
| | CXCL11 | C-X-C motif chemokine ligand 11 |
| | CXCR4 | C-X-C motif chemokine receptor 4 |
| | CYP27A1 | |
| | DTX1 | deltex E3 ubiquitin ligase 1 |
| | DUSP1 | dual specificity phosphatase 1 |
| | ERRFI1 | ERBB receptor feedback inhibitor 1 |
| | F3 | coagulation factor III, tissue factor |
| | FGFR3 | fibroblast growth factor receptor 3 |
| | GATA2 | GATA binding protein 2 |
| | GGT1 | gamma-glutamyltransferase 1 |
| | HAVCR1 | hepatitis A virus cellular receptor 1 |
| | HCLS1 | hematopoietic cell-specific Lyn substrate 1 |
| | HGFAC | HGF activator |
| | IDI1 | isopentenyl-diphosphate delta isomerase 1 |
| | IGFBP3 | insulin like growth factor binding protein 3 |
| | INPPL1 | inositol polyphosphate phosphatase like 1 |
| | JUND | JunD proto-oncogene, AP-1 transcription |
| | | factor subunit |
| | MAFK | MAF bZIP transcription factor K |
| | NEU1 | neuraminidase 1 |
| | NFIL3 | nuclear factor, interleukin 3 regulated |
| | NFKB1 | nuclear factor kappa B subunit 1 |
| | NKX3-1 | NK3 homeobox 1 |
| | NR3C1 | nuclear receptor subfamily 3 group C |
| | | member 1 |
| | PLEC | plectin |
| | PRDM1 | PR/SET domain 1 |
| | SAT1 | spermidine/spermine N1-acetyltransferase 1 |
| | SMAD6 | SMAD family member 6 |
| | THBS1 | |
| | TNFRSF14 | TNF receptor superfamily member 14 |
| | TNFSF10 | TNF superfamily member 10 |
| | VEGFC | vascular endothelial growth factor C |
| RELA | NFKB1 | nuclear factor kappa B subunit 1 |
| RNASEH2B | DDX58 | RNA sensor RIG-I |
| RXRA | TCF7 | transcription factor 7 |
| SMAD4 | ACVR1C | activin A receptor type 1C |
| | BGLAP | bone gamma-carboxyglutamate protein |
| | BMI1 | BMI1 proto-oncogene, polycomb ring finger |
| | CCN2 | cellular communication network factor 2 |
| | CCNE1 | cyclin E1 |
| | CCNG2 | cyclin G2 |
| | CDK6 | cyclin dependent kinase 6 |
| | CDKN1A | cyclin dependent kinase inhibitor 1A |
| | COL1A2 | |
| | CSF1 | colony stimulating factor 1 |
| | CXCL3 | C-X-C motif chemokine ligand 3 |
| | DAPK1 | death associated protein kinase 1 |
| | EPB41L5 | erythrocyte membrane protein band 4.1 |
| | | like 5 |
| | FOSL1 | FOS like 1, AP-1 transcription factor |
| | | subunit |
| | FST | follistatin |
| | FSTL3 | follistatin like 3 |
| | GADD45B | growth arrest and DNA damage inducible |
| | | beta |
| | GAST | gastrin |
| | GUCY2F | guanylate cyclase 2F, retinal |
| | HMCN1 | hemicentin 1 |
| | HMOX1 | heme oxygenase 1 |
| | ID2 | inhibitor of DNA binding 2 |
| | IER3 | immediate early response 3 |
| | IL31 | interleukin 31 |
| | ITGB7 | integrin subunit beta 7 |
| | KITLG | KIT ligand |
| | MUC4 | mucin 4, cell surface associated |
| | NUP153 | nucleoporin 153 |
| | PDGFB | platelet derived growth factor subunit B |
| | PILRA | paired immunoglobin like type 2 receptor |
| | | alpha |
| | SERTAD1 | SERTA domain containing 1 |
| | SMAD3 | SMAD family member 3 |
| | SMAD7 | SMAD family member 7 |
| | SPI1 | Spi-1 proto-oncogene |
| | SSTR2 | somatostatin receptor 2 |
| | STAR | steroidogenic acute regulatory protein |
| | TERT | telomerase reverse transcriptase |
| | THBS1 | |
| | TIMP3 | |
| | TNFRSF10A | TNF receptor superfamily member 10a |
| | ZFP36 | ZFP36 ring finger protein |
| SS18 | CABP7 | calcium binding protein 7 |
| | CACNA1G | calcium voltage-gated channel subunit |
| | | alpha1 G |
| | CBX4 | chromobox 4 |
| | GADD45B | growth arrest and DNA damage inducible |
| | | beta |
| | GCH1 | GTP cyclohydrolase 1 |
| | MSX1 | msh homeobox 1 |
| | NFATC1 | nuclear factor of activated T cells 1 |
| | NGFR | nerve growth factor receptor |
| | PDGFB | platelet derived growth factor subunit B |
| | SLC34A2 | solute carrier family 34 member 2 |
| | TENT5C | terminal nucleotidyltransferase 5C |
| TGFB1 | LIF | LIF interleukin 6 family cytokine |
| TGM2 | BCL6 | BCL6 transcription repressor |
| | BTG2 | BTG anti-proliferation factor 2 |
| | CCRL2 | C-C motif chemokine receptor like 2 |
| | CDK6 | cyclin dependent kinase 6 |
| | CEACAM1 | CEA cell adhesion molecule 1 |
| | CFP | complement factor properdin |
| | CSRNP1 | cysteine and serine rich nuclear |
| | | protein 1 |
| | DAPK2 | death associated protein kinase 2 |
| | DNAAF2 | dynein axonemal assembly factor 2 |
| | E2F5 | E2F transcription factor 5 |
| | HK3 | hexokinase 3 |
| | ITGAM | integrin subunit alpha M |
| | ITGAX | integrin subunit alpha X |
| | ITGB3 | |
| | LGALS9 | galectin 9 |
| | MAFB | MAF bZIP transcription factor B |
| | MDN1 | midasin AAA ATPase 1 |
| | MORC2 | MORC family CW-type zinc finger 2 |
| | NLRC4 | NLR family CARD domain containing 4 |
| | OAS2 | 2′-5′-oligoadenylate synthetase 2 |
| | PLEKHO2 | pleckstrin homology domain containing O2 |
| | RFLNB | refilin B |
| | SEMA6B | |
| | SEMA7A | semaphorin 7A (John Milton Hagen blood |
| | | group) |
| | SERPINB2 | serpin family B member 2 |
| | SIGLEC12 | sialic acid binding Ig like lectin 12 |
| | SLFN5 | schlafen family member 5 |
| | THBS1 | |
| | TNFAIP2 | TNF alpha induced protein 2 |
| | TP63 | tumor protein p63 |
| | UBA7 | ubiquitin like modifier activating |
| | | enzyme 7 |
| | WDR3 | WD repeat domain 3 |
| TP53 | LIF | LIF interleukin 6 family cytokine |
| | NFKB1 | nuclear factor kappa B subunit 1 |
| TP73 | FGFR3 | fibroblast growth factor receptor 3 |
| TRPC1 | APAF1 | apoptotic peptidase activating factor 1 |
| | CDK6 | cyclin dependent kinase 6 |
| | CDKN1A | cyclin dependent kinase inhibitor 1A |
| | TRPC1 | transient receptor potential cation |
| | | channel subfamily C member 1 |
| IKBKB | CCND1 | cyclin D1 |
| KAT5 | CCND1 | cyclin D1 |
| SYVN1 | CCND1 | cyclin D1 |
| ATF4 | HSP90B1 | heat shock protein 90 beta family |
| | | member 1 |
| C5 | NFKBIA | NFKB inhibitor alpha |
| CD36 | LIF | LIF interleukin 6 family cytokine |
| CDK9 | NFKBIA | NFKB inhibitor alpha |
| CHD1 | LIF | LIF interleukin 6 family cytokine |
| CSF1 | HSP90B1 | heat shock protein 90 beta family |
| | | member 1 |
| ECSIT | NFKBIA | NFKB inhibitor alpha |
| FN1 | NFKBIA | NFKB inhibitor alpha |
| FOXL2 | LIF | LIF interleukin 6 family cytokine |
| GPER1 | NFKBIA | NFKB inhibitor alpha |
| IL18 | LIF | LIF interleukin 6 family cytokine |
| IL6 | ADAMTS1 | ADAM metallopeptidase with |
| | | thrombospondin type 1 motif 1 |
| | ADAMTS5 | ADAM metallopeptidase with |
| | | thrombospondin type 1 motif 5 |
| | CEBPB | CCAAT enhancer binding protein |
| | | beta |
| | CEBPD | |
| | CISH | |
| | CXCL8 | C-X-C motif chemokine ligand 8 |
| | CYP1B1 | cytochrome P450 family 1 subfamily B |
| | | member 1 |
| | FGB | fibrinogen beta chain |
| | FLI1 | |
| | GLI1 | |
| | HGF | |
| | HPGD | |
| | HSPA5 | heat shock protein family A (Hsp70) |
| | | member 5 |
| | IL1RL1 | |
| | IL6 | |
| | ITGB3 | |
| | KIT | |
| | LAG3 | |
| | LDLR | low density lipoprotein receptor |
| | MAP2 | |
| | NFKBIA | NFKB inhibitor alpha |
| | PGF | |
| | PLEK | |
| | PPBP | |
| | PTGS2 | prostaglandin-endoperoxide synthase 2 |
| | RORC | |
| | RUNX2 | |
| | S1PR3 | |
| | STAP2 | signal transducing adaptor family |
| | | member 2 |
| | TAL1 | |
| | TBC1D9 | TBC1 domain family member 9 |
| | THBS1 | |
| | TNFRSF25 | TNF receptor superfamily member 25 |
| JUN | NFKBIA | NFKB inhibitor alpha |
| KLF2 | NFKBIA | NFKB inhibitor alpha |
| KLF6 | NFKBIA | NFKB inhibitor alpha |
| NPM1 | LIF | LIF interleukin 6 family cytokine |
| PRKCD | LIF | LIF interleukin 6 family cytokine |
| | NFKBIA | NFKB inhibitor alpha |
| | TRPC1 | |
| RAF1 | LIF | LIF interleukin 6 family cytokine |
| RELA | HSP90B1 | heat shock protein 90 beta family |
| | | member 1 |
| | NFKBIA | NFKB inhibitor alpha |
| RIPK2 | NFKBIA | NFKB inhibitor alpha |
| SP110 | TCF7 | |
| STAT3 | LIF | LIF interleukin 6 family cytokine |
| STAT6 | LIF | LIF interleukin 6 family cytokine |
| | TCF7 | |
| TGFB1 | HSP90B1 | heat shock protein 90 beta family |
| | | member 1 |
| | LIF | LIF interleukin 6 family cytokine |
| | NFKBIA | NFKB inhibitor alpha |
| THEM6 | HSP90B1 | heat shock protein 90 beta family |
| | | member 1 |
| TP53 | LIF | LIF interleukin 6 family cytokine |
| | NFKBIA | NFKB inhibitor alpha |
| XBP1 | HSP90B1 | heat shock protein 90 beta family |
| | | member 1 |

In certain embodiments, the expression of at least one gene is reduced and/or expression of at least one gene is increased in the treatment cell line or treatment culture condition, as compared to the control cell line or control culture condition, to generate an engineered or modified cell line as disclosed herein.

In certain embodiments, the engineered or modified cell line or the treatment cell line is a mammalian cell line or an insect cell line. In certain embodiments, the mammalian cell line is a CHO cell line. In certain embodiments, the mammalian cell line is a Vero cell line. In certain embodiments, the mammalian cell line is a HeLa cell line. In certain embodiments, the mammalian cell line is a MDCK cell line. In certain embodiments, the mammalian cell line is a BHK cell line. In certain embodiments, the mammalian cell line is a A549 cell line. In certain embodiments, the mammalian cell line is an amniocyte cell line. In certain embodiments, the mammalian cell line is a HEK293 cell line. In certain embodiments, the insect cell line is an Sf9 cell line or a Hi5 cell line.

In certain embodiments, the cell line is an engineered or modified host cell line for the production of viral particles. In certain embodiments, the cell line is a producer cell line.

In certain embodiments, the engineered or modified cell line is stably transfected with nucleic acid sequences encoding AAV Rep and/or Cap and a gene of interest (e.g., a transgene) flanked by inverted terminal repeat (ITR) sequences, to generate an engineered or modified producer cell line. As such, in certain embodiments, the engineered or modified producer cell line comprises stably integrated nucleic acid sequences encoding AAV Rep and/or Cap and a gene of interest (e.g., a transgene) flanked by inverted terminal repeat (ITR) sequences. In certain embodiments, the engineered or modified cell line is transiently transfected with nucleic acid sequences encoding AAV Rep and/or Cap and a gene of interest (e.g., a transgene) flanked by inverted terminal repeat (ITR) sequences, to generate an engineered or modified producer cell line. As such, in certain embodiments, the engineered or modified cell line comprises transient (e.g., not stably integrated) nucleic acid sequences encoding AAV Rep and/or Cap and a gene of interest (e.g., a transgene) flanked by inverted terminal repeat (ITR) sequences. In certain embodiments, the sequences are encoded on one plasmid. In certain embodiments, the sequences are on more than one plasmid.

### Recombinant HEK293 AAV Producer Cell Lines and Methods Thereof

In certain embodiments, the present disclosure provides compositions and methods for generating a HEK293 producer cell line used for rAAV production. In certain embodiments, the compositions and methods comprise alteration of the expression of glyceraldehyde-3-phosphate dehydrogenase (GAPDH) in a host cell line to generate an engineered or modified producer HEK293 cell line.

As used herein, the term “glyceraldehyde-3-phosphate dehydrogenase” or “GAPDH” refers to the gene encoding the protein glyceraldehyde-3-phosphate dehydrogenase. The human GAPDH gene corresponds to NCBI gene accession number NG_007073.2. Glyceraldehyde-3-phosphate dehydrogenase catalyzes the reaction of glyceraldehyde-3-phosphate to D-glycerate 1,3-bisphosphate.

In certain embodiments, provided herein is an engineered or modified HEK293 producer cell line produced by a method comprising knocking down the expression or activity of GAPDH.

In certain embodiments, the expression of GAPDH is reduced in the treatment HEK293 cell line or treatment culture condition, as compared to the control HEK293 cell line or control culture condition.

In certain embodiments, the HEK293 cell line is a host cell line for the production of viral particles. In certain embodiments, the cell line is a HEK293 producer cell line.

In certain embodiments, the engineered or modified HEK293 host cell line is stably transfected with nucleic acid sequences encoding AAV Rep and/or Cap and a gene of interest (e.g., a transgene) flanked by inverted terminal repeat (ITR) sequences, to generate an engineered or modified HEK293 producer cell line. As such, in certain embodiments, the engineered or modified HEK293 producer cell line comprises stably integrated nucleic acid sequences encoding AAV Rep and/or Cap and a gene of interest (e.g., a transgene) flanked by inverted terminal repeat (ITR sequences. In certain embodiments, the engineered or modified HEK293 host cell line is transiently transfected with nucleic acid sequences encoding AAV Rep and/or Cap and a gene of interest (e.g., a transgene) flanked by inverted terminal repeat (ITR) sequences, to generate an engineered or modified HEK293 host cell line comprises transient (e.g., not stably integrated) nucleic acid sequences encoding AAV Rep and/or Cap and a gene of interest (e.g., a transgene) flanked by inverted terminal repeat (ITR) sequences. In certain embodiments, the sequences are encoded on one plasmid. In certain embodiments, the sequences are on more than one plasmid.

### Helper Nucleic Acid Molecules

In certain embodiments, the producer cell line further comprises AAV helper function encoding sequences of an AAV helper virus. These sequences include helper virus sequences necessary for AAV replication, such as an adenovirus (Ad) or herpesvirus. In certain embodiments, the AAV helper virus is an adenovirus. In certain embodiments, the adenovirus is Ad5 or Ad2. In certain embodiments, the adenovirus is wild type Ad5 or wild type Ad2. In certain embodiments, the AAV helper virus is a herpes simplex virus (HSV).

Adenovirus (Ad) helper viruses are small, non-enveloped viruses. The Ad genome encodes about 39 genes, which are classified as either early or late depending on whether they are expressed before or after DNA replication. Helper functions for AAV expression are provided by early Ad transcription units encoding proteins E1a, E1b, E2a, E4, and the transcription unit VARNA. Major late gene products are encoded in the transcription units L1 to L5. The L4 region encodes: L4-100K, a translation enhancer protein that targets specific late mRNAs, protein pVIII, a structural protein of the viral capsid, L4-22K, required for viral DNA packaging into the empty capsid, and splicing factor L4-33K.

In certain embodiments, the helper genes comprise at least one of: E1a, E1b, E2a, L4, E4, and VA. Helper genes can be present in a vector, in an adenovirus, or integrated into a cell genome.

In certain embodiments, Ad nucleic acid molecules encoding helper genes can be present in an Ad genome and/or encapsidated in an Ad vector. In certain embodiments, the helper genes can be an isolated molecule or inserted into one or more expression cassettes, vectors or plasmids.

### Nucleic Acid Molecules Encoding AAV Rep/Cap Proteins

A Rep coding nucleic acid molecule can encode one or more AAV Rep proteins. In certain embodiments, a Rep coding nucleic acid molecule encodes Rep proteins that are necessary for viral genome replication and packaging into new virions. In certain embodiments, a Rep coding nucleic acid molecule can encode one or more of AAV Rep78, Rep68, Rep52, and Rep40. In certain embodiments, a Rep coding nucleic acid molecule can encode at least Rep78 or Rep68 and Rep52 or Rep40. In certain embodiments, a Rep coding nucleic acid molecule can encode Rep78 and Rep52 and/or Rep40 protein or Rep68 and Rep52 and/or Rep40. In certain embodiments, a Rep coding nucleic acid molecule can encode Rep78, Rep68, Rep52 and Rep40 proteins.

A Rep coding nucleic acid molecule can include a wild type or synthetic nucleic acid sequence encoding one or more AAV Rep proteins. A Rep coding nucleic acid molecule can be derived from any AAV, including but not limited to serotypes AAV1, AAV2, AAV3a, AAV3b, AAV4, AAV5, AAV6, AAV7, AAV8, AAV9, AAV10, AAV11, or AAV13, or any other AAV, such as a chimeric AAV or a variant AAV. A Rep molecule can include a wild-type sequence modified by insertion, deletion, truncation and/or missense mutations.

In certain embodiments, a Rep coding nucleic acid molecule can be inserted into one or more expression cassettes, vectors or plasmids as described above. In certain embodiments, Ad nucleic acid molecules and a Rep coding nucleic acid molecule can be present on a single construct (e.g., plasmid backbone). In certain embodiments, a Rep coding nucleic acid molecule can be stably integrated into a genome of a cell line for AAV production.

A Cap coding nucleic acid molecule can encode one or more AAV Cap proteins. A Cap coding nucleic acid molecule can encode one or more AAV capsid subunits. In certain embodiments, a Cap coding nucleic acid molecule encodes a sufficient number of capsid subunits for producing a functional AAV capsid. AAV capsid structure is described in BERNARD N. FIELDS et al., VIROLOGY, volume 2, chapters 69 & 70 (4th ed., Lippincott-Raven Publishers).

A Cap coding nucleic acid molecule can include a wild-type or synthetic nucleic acid molecule encoding one or more AAV Cap proteins. In certain embodiments, a Cap coding nucleic acid molecule can include one or more of VP1, VP2, and VP3. A Cap coding nucleic acid molecule can be derived from any AAV, including but not limited to serotypes AAV1, AAV2, AAV3a, AAV3b, AAV4, AAV5, AAV6, AAV7, AAV8, AAV9, AAV10, AAV11, or AAV13, or any other AAV, such as a chimeric AAV or a variant AAV. A Cap coding nucleic acid molecule can include a wild type sequence modified by insertion, deletion, truncation and/or missense mutations. A Cap coding nucleic acid molecule can be modified to provide an altered tropism. In certain embodiments, a Cap coding nucleic acid molecule encodes a capsid protein for transducing a specific cell, e.g., CNS, heart, liver, lung, pancreas, photoreceptor cells, retinal pigment epithelium, and/or skeletal muscle.

In certain embodiments, a Cap coding nucleic acid molecule can be present in a helper Ad genome and/or an Ad vector. In certain embodiments, a Cap coding nucleic acid molecule can be encapsidated in an Ad vector. In certain embodiments, a Rep coding nucleic acid molecule and a Cap coding nucleic acid molecule can be present on a single construct (e.g., plasmid backbone). In certain embodiments, a Rep coding nucleic acid molecule and a Cap coding nucleic acid molecule can be encapsidated in an Ad vector. A Cap coding nucleic acid molecule and a Rep coding nucleic acid molecule can encode proteins from the same AAV type, or from different AAV types. In certain embodiments, a Rep coding molecule, a Cap coding nucleic acid molecule and Ad nucleic acid molecules can be present on a single vector or a single expression cassette. In certain embodiments, a Cap coding nucleic acid molecule can be stably integrated into a genome of a cell line for AAV production.

In certain embodiments, a Rep coding nucleic acid molecule and a Cap coding nucleic acid molecule can be present on a single Rep-Cap plasmid. A Rep-Cap plasmid can include an AAV promoter to control expression of AAV Rep and Cap proteins described above. A promoter can be any desired promoter, selected based on known considerations, such as the level of expression of a nucleic acid molecule functionally linked to the promoter and the cell type in which the vector is to be used. A promoter can be tissue/cell-specific. A promoter can be a prokaryotic, eukaryotic, fungal, nuclear, mitochondrial, viral, or plant promoter. A promoter can be exogenous or endogenous to the cell type being transduced by a vector. Promoters can be selected from, for example, bacterial promoters, known strong promoters such as SV40 or the inducible metallothionein promoter. A promoter can be a promoter of any AAV serotypes (e.g., an AAV P5 promoter). A promoter can be a chimeric regulatory promoter for targeted gene expression or promoter derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), and the like. Non-limiting examples of suitable plasmid backbones for a Rep-Cap plasmid can include pHLP19, pETC18, pETC19, and pAAV-RC2.

In certain embodiments, the engineered or modified cell line produces a titer of rAAV higher than a wild-type parental cell line. In certain embodiments, the titer of rAAV is about 1.1-, about 1.2-, about 1.3-, about 1.4-, about 1.5-, about 1.6-, about 1.7-, about 1.8-, about 1.9-, about 2-, about 2.5-, about 3-, about 3.5-, about 4-, about 4.5-, about 5-, about 5.5-, about 6-, about 6.5-, about 7-, about 7.5-, about 8-, about 8.5-, about 9-, about 9.5-, about 10-, about 10.5-, about 11-, about 11.5-, about 12-, about 12.5-, about 13-, about 13.5-, about 14-, about 14.5-, about 15-, about 20-, about 30-, about 40-, about 50-, about 60-, about 70-, about 80-, about 90-, about 100-, about 200-, about 500-, about 1000-fold higher compared to the tier of rAAV produced from a cell line in which the expression of the gene is not altered. In certain embodiments, the engineered or modified cell line (e.g., producer cell line) produces a titer of rAAV at least about 1.5-fold higher compared to the titer of rAAV produced from a cell line in which the expression of the gene is not altered (e.g., a wild-type parental cell line).

In certain embodiments, as provided herein, is a method for identifying a gene associated with increasing the production of rAAV particles from a cell as described herein, wherein the method comprises culturing the cell in the presence of a modulator that alters the expression or activity of at least one gene selected from the group consisting of: ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, and PARD6G-AS1, a transcription factor gene listed in Tables 1-4, and a transcription factor targeted gene listed in Tables 1-4, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2.

In certain embodiments, as provided herein, is a method of modulating gene expression in a cell line as provided herein, wherein the method comprises culturing the cell in the presence of a modulator that alters the expression or activity of at least one gene selected from the group consisting of: ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, and PARD6G-AS1, a transcription factor gene listed in Tables 1-4, and a transcription factor targeted gene listed in Tables 1-4, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2.

In certain embodiments, as provided herein, is a method for increasing the production of rAAV particles, wherein the method comprises culturing the cells as provided herein, in the presence of a modulator, wherein the expression or activity of at least one of the following genes: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, TNFAIP3, EGFR, IGF1R, INSR, and GRB2 is reduced as compared to a control cell line, and/or in which the expression of at least one of ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1 is increased as compared to a control cell line, under conditions that allow for production and/or secretion of the recombinant viral particles.

In certain embodiments, as provided herein, is a method of modulating gene expression in a cell line as provided herein for increasing the production of rAAV particles from the cell, wherein the method comprises culturing the cell in the presence of a modulator that alters the expression or activity of at least one gene selected from the group consisting of: ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, FAM46C, HHEX, TXNIP, ASNS, PLEKH02, CDKN1A, C5orf64, COL2A1, GJB3, ISYNA1, PARD6G-AS1, RP11-268J15.6, a transcription factor gene listed in Tables 1-4, and a transcription factor targeted gene listed in Tables 1-4, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2.

In certain embodiments, as provided herein, is a method for increasing the production of rAAV particles, wherein the method comprises culturing a cell as provided herein, in the presence of a modulator, wherein the expression or activity of at least one of the following genes: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3 is reduced as compared to a control cell line, and/or in which the expression of at least one of ASNS, BCL2, CDKN1A, COL20A1, FAM46C, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1 is increased as compared to a control cell line, under conditions that allow for production and/or secretion of the recombinant viral particles.

In certain embodiments, as provided herein, is a method for identifying a gene associated with increasing the production of rAAV particles from a HEK293 cell as described herein, wherein the method comprises culturing the HEK293 cell in the presence of a modulator that alters the expression or activity of glyceraldehyde-3-phosphate dehydrogenase (GAPDH).

In certain embodiments, as provided herein, is a method of modulating gene expression in the HEK293 cell line as provided herein, wherein method comprises culturing the HEK293 cell in the presence of a modulator that alters the expression or activity of glyceraldehyde-3-phosphate dehydrogenase (GAPDH).

In certain embodiments, as provided herein, is a method of modulating gene expression in a HEK293 cell line as provided herein for increasing the production of rAAV particles from the HEK293 cell, wherein the method comprises culturing the HEK293 cell in the presence of a modulator that alters the expression or activity of glyceraldehyde-3-phosphate dehydrogenase (GAPDH).

In certain embodiments, as provided herein, is a method for increasing the production of rAAV particles, wherein the method comprises culturing a HEK293 cell as provided herein, in the presence of a modulator, wherein the expression or activity of glyceraldehyde-3-phosphate dehydrogenase (GAPDH), under conditions that allow for production and/or secretion of the recombinant viral particles.

### AAV Constructs

Adeno-associated viruses (AAVs) are small, non-pathogenic viruses of the Parvoviridae family. To date, numerous serologically distinct AAVs have been identified, and more than a dozen have been isolated from humans or primates. AAV is distinct from other members of this family by its dependence upon a helper virus for replication.

AAV genomes possess a broad host range, transduce both dividing and non-dividing cells in vitro and in vivo, and maintain high levels of expression of the transduced genes. AAV particles are generally heat stable. AAV particles are also resistant to solvents, detergents, changes in pH, and temperature. They may also be column purified and/or concentrated on gradients (e.g., CsCl gradients) or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short, inverted terminal repeats (ITRs) that can fold into hairpin structures and serve as the origin of viral DNA replication.

The AAV virion or particle is a non-enveloped, icosahedral particle approximately 25 nm in diameter that comprises an AAV capsid. The AAV particle comprises an icosahedral symmetry comprised of three related capsid proteins-VP1, VP2, and VP3-which together form the capsid. The genome of most native AAVs often contain two open reading frames (ORFs), sometimes referred to as a left ORF and a right ORF. The right ORF often encodes the capsid proteins VP1, VP2, and VP3. The VP1, VP2, and VP3 capsid proteins differ from each other by the use of alternative splicing and an unusual start codon. The left ORF often encodes the non-structural Rep proteins which are involved in regulation of replication and transcription in addition to the often encodes the non-structural Rep proteins, Rep 40, Rep 52, Rep 68, and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. The genome of an AAV (e.g., an rAAV) may encode some or all of the Rep proteins. The genome of an AAV (e.g., an rAAV) may not encode the Rep proteins. One or more of the Rep proteins can be delivered in trans and are therefore not included in an AAV particle comprising a nucleic acid encoding a recombinant polypeptide.

### AAV Constructs

In some embodiments, the AAV vectors or expression cassettes of the present disclosure are obtained by cloning at least one heterologous nucleic acid molecule of interest (i.e., a transgene) into an AAV construct. For example, an AAV vector or expression cassette can include recombinant DNA containing a heterologous nucleic acid molecule, such as a transgene, flanked by ITR sequences for packaging into an AAV capsid. An AAV vector or expression cassette can be introduced into a eukaryotic cell line for producing AAV.

An AAV vector or expression cassette can be constructed using techniques for providing operatively linked components in the direction of transcription, control elements including a transcriptional initiation region, the DNA of interest, and a transcriptional termination region. The control elements can be selected to be functional in a mammalian cell. Termination signals, such as polyadenylation sites, can be included in the plasmid.

In certain embodiments, the nucleic acid sequences as disclosed herein, comprise AAV ITR sequences. These ITR sequences are located 5′ and 3′ to a polynucleotide sequence. The AAV ITR sequences can include any cis elements required for packaging allowing for production of recombinant AAV.

The nucleic acid can include ITR sequences from any AAV serotype. Nucleic acid molecules of AAV ITR regions are known. An ITR can have a wild type nucleic acid sequence, or a modified sequence, e.g., by the insertion, deletion or substitution of nucleotides. An AAV ITR can be derived from any AAV serotype, including without limitation, AAV1, AAV2, AAV3a, AAV3b, AAV4, AAV5, AAV6, AAV7, AAV8, AAV9, AAV10, AAV11, or AAV13, and 5′ and 3′ ITRs in an AAV vector can be independently selected therefrom.

The nucleic acid can include a polynucleotide of any length positioned within two AAV ITR sequences (i.e., a polynucleotide can be flanked by ITR sequences). A polynucleotide can be a transgene of at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000 or at least 10000 nucleotides. In certain embodiments, an ITR flanked polynucleotide sequence does not exhibit biological activity. For example, enhancers, promoters, splicing regulators, noncoding RNAs, antisense sequences, and/or coding sequences can be absent from a polynucleotide sequence positioned within two AAV ITR sequences. In certain embodiments, each of enhancers, promoters, splicing regulators, noncoding RNAs, antisense sequences, and coding sequences are absent. In certain embodiments, a polynucleotide sequence positioned within two AAV ITR sequences does not include an open reading frame.

The AAV vector or expression cassettes can include recombinant DNA containing a heterologous nucleic acid molecule, such as a transgene, flanked by ITR sequences for packaging into an AAV capsid. An AAV vector or expression cassette can be introduced into a eukaryotic cell line for producing AAV.

An AAV vector or expression cassette can be constructed using techniques for providing operatively linked components in the direction of transcription, control elements including a transcriptional initiation region, the DNA of interest, and a transcriptional termination region. The control elements can be selected to be functional in a mammalian cell. Termination signals, such as polyadenylation sites, can be included in the plasmid.

A heterologous nucleic acid molecule of an AAV vector or expression cassette can include a polynucleotide with biological activity of an anti-sense RNA molecule, shRNA, miRNA, a ribozyme, or a gene encoding a polypeptide of interest, and optionally one or more a nucleic acid sequences capable of directing expression of a particular heterologous nucleic acid sequence in an appropriate host cell (e.g., mammal). Non-limiting of nucleic acid sequences capable of directing expression include a promoter and termination signal. In certain embodiments, a heterologous nucleic acid molecule can be included in a chimeric expression cassette. In certain embodiments, an expression cassette can be naturally occurring, optionally obtained in a recombinant form for heterologous expression.

In certain embodiments, a heterologous nucleic acid molecule can be at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000 nucleotides, at least 5000 nucleotides, at least 6000 nucleotides, at least 7000 nucleotides, at least 7500 nucleotides, or at least 8000 nucleotides.

In certain embodiments, a heterologous nucleic acid molecule (e.g., a transgene) can encode a polypeptide such as, but not limited to, a clotting factor, an enzyme, an antibody or other polypeptide of interest. In certain embodiments, a heterologous nucleic acid molecule can encode an RNA having a structural or therapeutic function such as, but not limited to, an antisense, siRNA, shRNA, miRNA, EGSs, gRNA, sgRNA, ribozyme, or aptamer.

In certain embodiments, a heterologous nucleic acid molecule can encode a peptide, polypeptide, or protein that binds to a specific target of interest, which can be useful for the treatment or prevention of disease in a subject. Examples of such heterologous nucleic acid molecules and associated peptides, polypeptides, or proteins include, but are not limited to, a gene encoding antibodies, MHC molecules, T-cell receptors, B-cell receptors, aptamers, avimers, receptor-binding ligands, or targeting peptides. Antibodies useful in the present disclosure can be monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion, humanized antibodies, and any other modified configuration of a immunoglobulin molecule including an antigen recognition site of the required specificity, including glycosylation variants of antibodies, amino acid sequence variants of antibodies, and covalently modified antibodies. Antibodies and specific binding fragments thereof can be murine, rat, human, or of any other origin (including chimeric or humanized antibodies). An antibody can include an antibody of any class, such as IgG, IgA, or IgM (or sub-class thereof), or an unspecified class.

A heterologous nucleic acid molecule comprising a gene of interest (e.g., a transgene) can encode a peptide, polypeptide, or protein that can be useful for the treatment or prevention of disease in a subject.

A heterologous nucleic acid molecule comprising a gene of interest (e.g., a transgene) can be a gene editing molecule used for modifying a genomic locus of interest (i.e., target) in a cell. A modification can include a disruption, deletion, repair, mutation, addition, alteration, or modification of a gene sequence at a target locus in a gene. Examples of gene-editing molecules include, but are not limited to, endonucleases such as zinc finger nucleases (ZFNs), transcription activator-like effector nucleases (TALENs), meganucleases, restriction endonucleases, recombinases, and Clustered Regularly Interspersed Short Palindromic Repeats (CRISPR)/CRISPR-associated (Cas) proteins.

In certain embodiments, the heterologous nucleic acid molecule comprising a gene of interest can be a double stranded RNA (dsRNA), a small interfering RNA (siRNA), a small hairpin RNA (shRNA), a microRNA (miRNA), or an antisense RNA oligonucleotide (ASO). In certain embodiments, the heterologous nucleic acid molecule comprising a gene of interest comprises a template for homologous arm mediated recombination.

In certain embodiments, an AAV vector or expression cassette can be multi-cistronic. A multi-cistronic vector or expression cassette can simultaneously express two or more (e.g., 2, 3, 4, 5, 6, or more) separate proteins from the same mRNA. In such cases, the multiple heterologous nucleic acid molecules can be separated by an element that allows for separate translation for each gene.

In certain embodiments, a large heterologous nucleic acid molecule can be split into more than one construct, whereby multiple constructs can express two or more fragments for assembly into a protein of interest in vivo.

In certain embodiments, AAV vectors or expression cassettes do not include a reporter gene or a selection marker. In certain embodiments, the transgene-containing vector or expression cassette does not comprise an antibiotic resistance gene. An AAV vector or expression cassette can include a stuffer sequence as described above.

### Genetic Modulators

In an aspect of the present disclosure, expression of at least one gene is reduced and/or expression of at least one gene is increased in an engineered or modified cell line, as compared to a control cell line. In certain embodiments, the cell line is modified by a modulator that increases or decreases the expression and/or activity of at least one gene. In certain embodiments, the cell line comprises a genetic modification. In certain embodiments, the modulator is a nuclease.

### Zinc Finger Nucleases

In certain embodiments, the nuclease is a zinc finger nuclease (ZFN). Targeted genetic engineering in many model systems and in human tissue culture cells has historically been challenging. The development of ZFNs dramatically changed the field. ZFNs are artificial endonucleases that consist of a designed zinc finger protein (ZFP) fused to the cleavage domain of the FokI restriction enzyme.

The DNA-binding domains of individual ZFNs typically contain between three and six individual zinc finger repeats and can each recognize between 9 and 18 basepairs. If the zinc finger domains perfectly recognize a 3 basepair DNA sequence, a 3-finger array may be generated that can recognize a 9 basepair target site.

The non-specific cleavage domain from the type II restriction endonuclease FokI is typically used as the cleavage domain in ZFNs. This cleavage domain must dimerize in order to cleave DNA, and thus a pair of ZFNs are required to target non-palindromic DNA sites. Standard ZFNs fuse the cleavage domain to the C-terminus of each zinc finger domain. To let the two cleavage domains dimerize and cleave DNA, the two individual ZFNs must bind opposite strands of DNA with their C-termini a certain distance apart. The most commonly used linker sequences between the zinc finger domain and the cleavage domain requires the 5′ edge of each binding site to be separated by 5 to 7 bp.

A ZFN may be redesigned to cleave new targets by developing ZFPs with new sequence specificities. For genome engineering, a ZFN is targeted to cleave a chosen genomic sequence. The cleavage event induced by the ZEN provokes a cellular repair process that in turn mediates efficient modification of the targeted genomic locus. The modification is dependent on how the cleavage event is repaired. For instance, if the ZFN-induced cleavage event is resolved via non-homologous end joining, this can result in small deletions or insertions, effectively leading to gene knockout. This approach has been used to establish easy and efficient reverse genetics that does not require selection in a number of model organisms. However, if the break is resolved via a homology-based process in the presence of an investigator-provided donor, small changes or entire transgenes can be transferred, often without selection, into a chromosome. This process is often referred to as a “gene correction” and “gene addition”. Using ZFNs in therapeutic applications require the engineering of ZFNs that are highly specific.

### Meganucleases

In certain embodiments, the nuclease is a meganuclease. Meganucleases are endodeoxyribonucleases characterized by a large recognition site (double-stranded DNA sequences of 12 to 40 base pairs). As a result, the recognition site generally occurs only once in any given genome. Among the meganucleases, the LAGLIDADG family of homing endonucleases has become a valuable tool for the study of genomes and genome engineering over the years. Meganucleases are thought of as “molecular DNA scissors” that can be used to replace, eliminate, or modify sequences in a highly targeted way. By modifying their recognition sequence through protein engineering, the targeted sequence can be changed. Meganucleases are used to modify all genome types, whether bacterial, plant or animal.

Meganucleases are mainly represented by two enzyme families collectively known as homing endonucleases: intron endonucleases and intein endonucleases. Introns propagate by intervening at a precise location in the DNA, where the expression of the meganuclease produces a break in the complementary intron- or intein-free allele.

The high specificity of meganucleases gives them a high degree of precision and lower cell toxicity than other naturally occurring restriction enzymes. Since being identified in the 1990s, subsequent work has shown that these enzymes are promising tools for genome engineering and gene editing, as they are able to efficiently induce homologous recombination, generate mutations, and alter reading frames.

### TALENS

In certain embodiments, the nuclease is a transcription activator-like effector nuclease (TALEN). TALENs are restriction enzymes that can be engineered to cut specific sequences of DNA. They are generally made by fusing a TAL effector DNA-binding domain to a DNA cleavage domain. Transcription activator-like effectors (TALEs) can be engineered to bind to practically any desired DNA sequence, so when combined with a nuclease (typically FokI), DNA can be cut at specific locations.

TAL effectors are proteins that are secreted by Xanthomonas bacteria via their type III secretion system when they infect plants. The DNA binding domain contains a repeated highly conserved 33-34 amino acid sequence with divergent 12^{th} and 13^{th} amino acids. These positions, referred to as the Repeat Variable Diresidue (RVD), are highly variable and show a strong correlation with specific nucleotide recognition. This relatively straightforward relationship between amino acid sequence and DNA recognition has allowed for the engineering of specific DNA-binding domains by selecting a combination of repeat segments containing the appropriate RVDs.

The DNA sequences encoding TALEN constructs can be inserted into plasmids. The cells targeted for genetic engineering are then transfected with the plasmids, and the gene products are expressed and enter the nucleus to access the genome. Alternatively, TALEN constructs can be delivered to the cells as mRNAs, thereby avoiding any potential genomic integration of the TALEN-expressing protein.

### CRISPR/Cas9

CRISPR RNA-guided genome engineering has revolutionized research into human genetic disease and many other aspects of biology. The most widely used genome editing tool is the type II-A Cas9 from Streptococcus pyogenes strain SF370 (SpCas9). Cas9 forms a ribonucleoprotein (RNP) complex with a CRISPR RNA (crRNA) and a trans-activating crRNA (tracrRNA) for efficient DNA cleavage both in bacteria and eukaryotes. The crRNA contains a guide sequence that directs the Cas9 RNP to a specific locus via base-pairing with the target DNA to form an R-loop. This process requires the prior recognition of a protospacer adjacent motif (PAM). R-loop formation activates the His-Asn-His (HNH) and RuvC-like endonuclease domains that cleave the target strand and the non-target strand of the DNA, respectively, resulting in a double-strand break (DSB). In some embodiments, the crRNA and tracrRNA are linked to form a single guide RNA (sgRNA). A CRISPR system described herein is composed of the CRISPR nuclease, or a polynucleotide encoding the nuclease, and a guide RNA, such as an sgRNA. In some embodiments, the Cas9 nuclease is a deactivated or dead Cas9 (dCas9). A dCas9 nuclease comprises one or more mutations that reduce or eliminate the nuclease activity of Cas9.

For mammalian applications, Cas9 and its guide RNAs can be expressed from DNA (e.g., a viral vector), RNA (e.g., Cas9 mRNA plus guide RNAs in a lipid nanoparticle), or introduced as a ribonucleoprotein (RNP). Viral delivery of Cas9 results and its guides can result in off-target editing, and viral vectors can elicit strong host immune responses (Mingozzi et al. Blood 122, 23-36 (2013)). RNA and RNP delivery platforms of Cas9 are suitable alternatives to viral vectors for many applications and have recently been shown to be effective genome editing tools in vivo (Yin et al. Nature Biotechnology 35, 1179 (2017); Lee et al. eLife 6, e25312 (2017)). RNP delivery of Cas9 also bypasses the requirement for Cas9 expression, leading to faster editing. Furthermore, Cas9 delivered as mRNA or RNP exists only transiently in cells and therefore exhibits reduced off-target editing. For instance, Cas9 RNPs were successfully used to correct hypertrophic cardiomyopathy (HCM) in human embryos without measurable off-target effects (Ma et al. Nature 548, 413 (2017).

### Small Molecules

In certain embodiments, the engineered or modified cell line or methods as provided herein comprise a modulator that is a small molecule. In certain embodiments, the modulator is DMSO. In certain embodiments, the modulator is DMF. In certain embodiments, the modulator is NMP. In certain embodiments, the modulator is dihydrolevoglucosenone (Cyrene). In certain embodiments, the DMSO used is at a concentration of at least 0.5%. In certain embodiments, the DMSO used is at a concentration of at least 1%. In certain embodiments, the engineered or modified cell line is cultured in the presence of an inhibitor of a gene as disclosed herein. In certain embodiments, the inhibitor inhibits at least one gene selected from the group consisting of: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3. In certain embodiments, the inhibitor comprises a synthetic small molecule inhibitor.

In certain embodiments, the engineered or modified cell line is cultured in the presence of an agonist of a gene as disclosed herein. In certain embodiments, the agonist is an agonist of at least one gene selected from the group consisting of: ASNS, BCL2, CDKN1A, COL20A1, FAM46C, GATA1, GJB3, LAS1L, LIF, NFKB2, NGEF, NME1, NR3C1, RPA1, SMAD4, TRPC1, TXNIP, PARD6G-AS1, PLEKHO2, ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1. In certain embodiments, the agonist comprises a synthetic small molecule agonist.

In certain embodiments, the engineered or modified HEK293 cell line or methods as provided herein comprise a modulator that is a small molecule. In certain embodiments, the modulator is DMSO. In certain embodiments, the modulator is DMF. In certain embodiments, the modulator is NMP. In certain embodiments, the modulator is dihydrolevoglucosenone (Cyrene). In certain embodiments, the DMSO used is at a concentration of at least 0.5%. In certain embodiments, the DMSO used is at a concentration of at least 1%. In certain embodiments, the engineered or modified HEK293 cell line is cultured in the presence of an inhibitor of a gene as disclosed herein. In certain embodiments, the inhibitor inhibits GAPDH. In certain embodiments, the inhibitor comprises a synthetic small molecule inhibitor. In certain embodiments, the inhibitor is DC-5163, 1,2,3,4,6-penta-O-galloyl-β-D-glucopyranose (PGG), or 4-octyl itaconate (4-OI). The inhibitor DC-5163 corresponds to CAS number 897771-47-0 and is described in further detail in Li et al. (Biorg Chem. 96:103620.2020). incorporated herein by reference.

### RNA-Based Modulators

In certain embodiments, the modulator is an RNA-based modulator. In certain embodiments, the modulator is a double stranded RNA (dsRNA). In certain embodiments, the modulator is a small interfering RNA (siRNA). In certain embodiments, the modulator is a small hairpin RNA (shRNA). In certain embodiments, the modulator is an antisense RNA oligonucleotide. In certain embodiments, the modulator is a micro RNA (miRNA).

In certain embodiments, the RNA-based modulator may comprise a substitution. In certain embodiments, the RNA-based modulator may comprise chemically modified nucleotides. In certain embodiments, the RNA-based modulator may comprise non-nucleotides (e.g., substitutions or modifications in the backbone, sugar chains, bases or nucleosides). These modifications may allow the RNA-based modulator to have an increased half-life in a subject or biological sample. In certain embodiments, the modifications improve the cellular uptake of the RNA-based modulator.

### DNA Integration of Expression Construct

In certain embodiments, increasing the expression or activity is carried out by transient delivery or stable integration of a target expression modulation construct into the host cell line.

In certain embodiments, increasing the expression or activity is carried out by introduction of an exogenous gene or expression cassette into a cell's DNA via homology-directed repair (HDR), through homologous recombination. HDR is a precise DNA repair process that uses a template to repair double-strand breaks (DSBs) in DNA. Supplementation of homologous DNA templates with site specific nucleases shifts the innate DNA repair pathway to HDR attaining targeted nucleotide changes. All HDR donor templates contain 5′ and 3′ homology sequences flanking the cut site, to exploit the HDR pathway. A wide range of viral derived or synthetically generated double-stranded and single-stranded HDR donor templates are currently tested for single nucleotide changes to large transgene insertion. Viral-derived HDR donors include integrase defective lentiviral vectors (IDLVs), adenovirus 5/35 serotype (AdVs), and adeno-associated vector (AAV) and single-stranded oligo deoxynucleotides (ssODNs) is a non-viral option.

In certain embodiments, the increase in expression or activity comprises a template for homologous arm mediated recombination. In certain embodiments, the modulator affects expression and/or activity of the target gene alone. In certain embodiments, the modulator affects expression and/or activity of the target gene in combination with another modulator as disclosed herein (e.g., CRISPR). In certain embodiments, the homologous arm mediated recombination results in gene knock-in. In certain embodiments, the homologous arm mediated recombination results in gene correction.

Homologous arm mediated recombination is one of the most commonly used methods to introduce a genetic mutation into the genome (e.g., gene knock-in). It allows desired and controlled genetic modifications in the genome. In certain embodiments, the homologous arm mediated recombination comprises a DNA repair template. In certain embodiments, the DNA repair template is in the form of a plasmid. In certain embodiments, the genetic modification is in an intron. In certain embodiments, the genetic modification is in an exon. In certain embodiments, the genetic modification is found in the promoter. In certain embodiments, the genetic modification is in an untranslated region (e.g., 5′ or 3′ UTR).

In certain embodiments, the increase in expression or activity comprises targeted integration, e.g., integration at a safe harbor locus. A number of safe harbor loci are known, and such a locus can be selected from the group consisting of but not limited to: AAVS1, ALB, Angptl3, ApoC3, ASGR2, CCR5, Rosa26, FIX, G6PC, Gys2, HGD, Lp(a), Pcsk9, Serpina1, TF, and TTR. Generally, disruption of these loci does not have an adverse effect on the cell, and robust transcription can be used to maintain the expression of an exogenously inserted gene. Insertion of the gene or gene modulation cassettes can be targeted to these safe harbor loci via homology-directed repair and/or CRISPR/Cas9.

In certain embodiments, the increase in expression or activity is due to random integration of a target expression modulation construct into the DNA of the host cell.

In certain embodiments, the increase in expression or activity is due to transposase-based recombination. Transposons are genetic elements that generally have modest target site selectivity and can thus insert themselves into many different DNA sites. In transposition, a specific enzyme known as transposase, is usually encoded by the transposon itself, and acts on a specific DNA sequence at each end of the transposon. It first disconnects it from the flanking DNA and then inserts it into a new target DNA site. There is no requirement for homology between the ends of the element and the insertion site.

In certain embodiments, the increase in expression or activity is due to an integrase mediated integration or a CRISPR-Cas activation method. In certain embodiments, the Cas comprises a dCas, such as dCas9.

### AAV Compositions and Methods of Use

AAV produced according to one or more embodiments of the present disclosure can be used for the delivery of nucleic acids to cells in vitro, ex vivo, and in vivo. AAV can be advantageously employed to deliver or transfer nucleic acids to animal cells, including mammalian cells.

In certain embodiments, a heterologous nucleic acid molecule can encode any polypeptide or RNA for production in a cell in vitro, ex vivo, or in vivo. For example, nucleic acids of interest include nucleic acids, such as genes, encoding polypeptides or RNAs. In certain embodiments, an AAV can be used to deliver a reporter, therapeutic (e.g., for medical or veterinary uses), immunogenic (e.g., for vaccines), or diagnostic polypeptide or RNA. In certain embodiments, an AAV can be used to introduce a heterologous nucleic acid sequence into cultured cells and the expressed gene product isolated therefrom.

An AAV produced according to the compositions and methods of the present disclosure can be used for delivering heterologous nucleic acids into a broad range of cells, including dividing and non-dividing cells. An AAV can be employed to deliver a nucleic acid of interest to a cell in vitro, e.g., to produce a polypeptide in vitro or for ex vivo gene therapy. An AAV can be useful in a method of delivering a nucleic acid to a subject in need thereof, e.g., to express an immunogenic or therapeutic polypeptide or a functional RNA in a subject. A subject can be in need of a beneficial effect provided by expression of an AAV delivered immunogenic or therapeutic polypeptide or a functional RNA. In certain embodiments, AAV can be used to produce a polypeptide of interest or functional RNA in cultured cells or in a subject to observe the effects of a polypeptide or functional RNA on a subject, for example, in connection with screening methods.

### EXAMPLES

The present disclosure is described and demonstrated by way of the following examples. However, the use of these and other examples anywhere in the specification is illustrative only and is not limiting.

### Example 1. DMSO Supplementation Enhances rAAV Production in Multiple Stable Producer Cells as Well as in Transiently Transfected Mammalian Host Cells

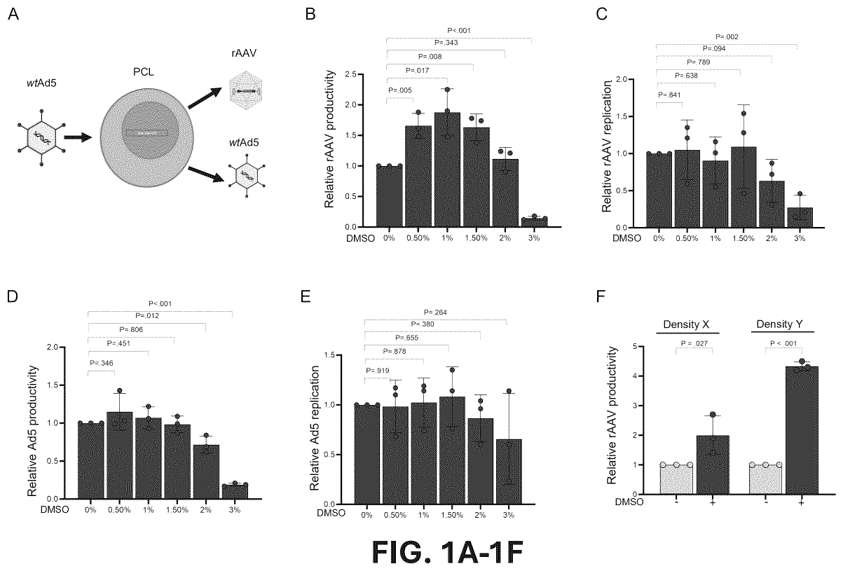

To assess the effect of DMSO on recombinant AAV (rAAV) vector production, experiments were performed in the context of both stable producer cell lines (PCL) and transiently transfected mammalian host cells. Production of rAAV in the PCLs was initiated by infection with a helper virus, such as wild type adenovirus type 5 (wtAd5) as shown in the schematic (FIG. 1A). For routine PCL infection, cells were seeded at the target cell density in a proprietary production media and infected with wtAd5 at a multiplicity of 100 DNase-resistant genomes (DRG) per cell. For infection in the presence of DMSO, producer cells were treated with 1% DMSO either three hours prior to wtAd5 infection (−3 h) or at the time of infection (0 h). Infections were performed in culture formats ranging from static 6-well plates, 96-well plates, suspension 24-deep well plates, suspension spin tubes or shake flasks and cultured at 37° C., 10% CO₂ for 48 or 72 hours before harvesting the culture (cells plus media) for analysis of AAV production.

In an experiment to determine the optimal concentration of DMSO for enhancement of rAAV production in the PCLs, DMSO was supplemented into production media at various concentrations (0, 0.5, 1, 1.5, 2, and 3%) at the time of infection (0 h). AAV vector production (FIG. 1B) and rAAV DNA replication (FIG. 1C) in PCL1 were analyzed in whole cell lysates at three days post infection. The impact of DMSO on wtAd5 virus production and adenoviral DNA replication were also assessed following supplementation, as shown in FIGS. 1D and 1E, respectively. The results of these experiments demonstrate that peak enhancement of rAAV production is achieved at the concentration of 1% DMSO. There is no significant increase in rAAV DNA replication at any of the DMSO concentrations tested. Likewise, DMSO supplementation did not result in an increase of either wtAd5 production or adenoviral DNA replication at any concentration.

The impact of DMSO on AAV productivity was subsequently assessed for wtAd5 infections performed at a higher cell density. PCL1 was infected with wtAd5 at two different cell densities X and Y (about four-fold higher density than X; X is the standard cell density) and rAAV productivity was assayed by qPCR. Titer values were normalized to the non-treated control for each infection cell density. The results showed that 1% DMSO supplementation at the time of infection increased AAV production for both the standard infection cell density X and four-fold higher cell infection density Y (FIG. 1F).

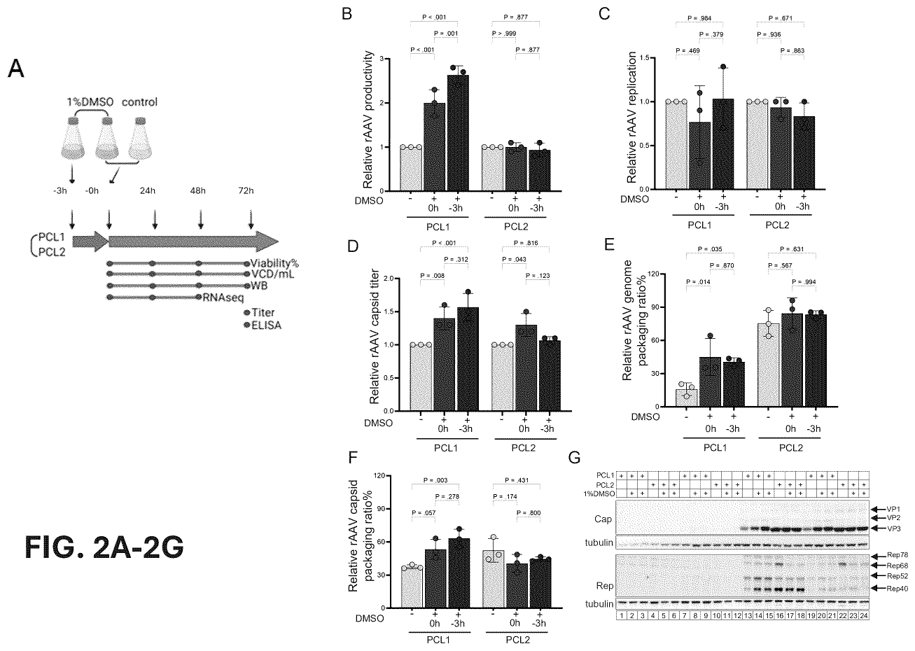

Supplementation of production medium with 1% DMSO and the impact on rAAV DNA replication and vector production was assessed in two different PCLs (PCL1 and PCL2) as shown in FIG. 2A. Addition of 1% DMSO at both 3 hr prior to infection (−3 h) and at the time of infection (0 h) increased AAV production from PCL1 but not PCL2 (FIG. 2B). To assess the biological factors contributing to the productivity increase, total vector genomes, total capsids, percent full capsids and rAAV genome packaging efficiency were analyzed in samples collected at 72 hr post-infection. rAAV genome packaging efficiency (FIG. 2E) represents the ratio of rAAV vector titer (i.e., packaged vector genomes) to total rAAV vector genomes (FIG. 2C) (tvg; i.e., total vector DNA copies) as assessed via qPCR. Total capsids were estimated via ELISA (FIG. 2D). Capsid packaging efficiency (% full capsids) was estimated as the ratio of rAAV vector titer to total capsids (FIG. 2F). DMSO treatment increased AAV capsid production, AAV vector genome packaging and capsid packaging efficiency in PCL1 but not PCL2, consistent with the effect on rAAV productivity in the former cell line.

Western blot analysis (FIG. 2G) was performed to evaluate the effect of DMSO on AAV Rep and Cap protein expression. Cell pellets collected at 0, 24, 48 and 72 hours post-infection (hpi) were lysed and subjected to electrophoresis and then transferred to a nitrocellulose membrane. Analysis of AAV Rep and Cap protein expression (FIG. 2G) showed that DMSO increased Cap protein and expression of Rep52/Rep40 in PCL1 but not in PCL2, consistent with AAV capsid production, capsid packaging efficiency and genome packaging results in PCL1. These results suggest that DMSO increases AAV production by improving both capsid production as well as the packaging of rAAV genomes into the capsid.

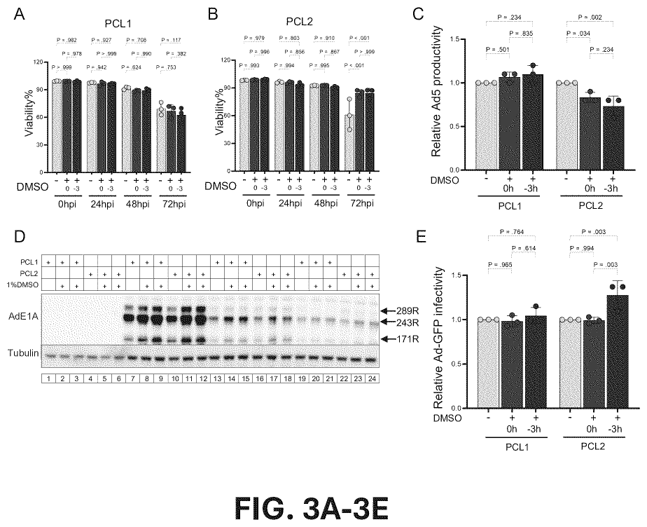

Additional parameters were evaluated following treatment of PCL1 and PCL2 with DMSO at both timepoints, 3 hours prior to infection (3 h) and at the time of infection (0 h). Cell viability was analyzed at 0 hpi, 24 hpi, 48 hpi, and 72 hpi. The results (FIGS. 3A and 3B) show that DMSO had no significant impact on percent viability in either PCL1 or PCL2; one exception is a trend toward an increase in viability at the 72 hpi timepoint in PCL2.

Because DMSO is known to increase cell permeability, DMSO might exert its effect on rAAV productivity by increasing the efficiency of infection of PCLs with wtAd5. To test this hypothesis, the impact of DMSO treatment on wtAd5 production (FIG. 3C) was assessed following infection of PCL1 and PCL2. Relative Ad5 productivity was determined by qPCR in whole cell lysates and the results were normalized to the control condition (no DMSO) for each PCL. Similar levels of wtAd5 production were observed in PCL1 in DMSO treated and control cultures but a trend towards reduced Ad5 production was observed in PCL2 with DMSO treatment (FIG. 3C). In addition, AdE1A protein expression was evaluated as a measure of wtAd5 infectivity. E1A protein levels were assessed via Western analysis in PCL1 and PCL2 at 0, 24, 48 and 72 hpi (FIG. 3D). DMSO treatment increased E1A expression in both PCL1 and PCL2. Infection of PCL1 and PCL2 with Ad5-GFP was also carried out to assess the impact of DMSO on infection efficiency (FIG. 3E). The percentage of cells expressing GFP was analyzed via Celigo at 24 hpi. The results suggested that DMSO had no impact on Ad5-GFP infection in PCL1 regardless of the time of treatment; however, DMSO appeared to increase Ad5-GFP infection in PCL2 when added at 3 hpi.

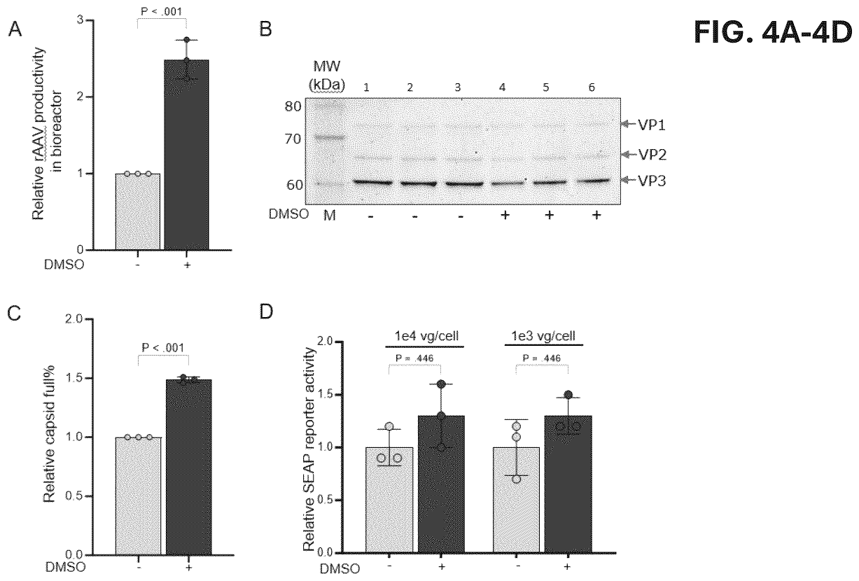

The effect of DMSO on vector quality was analyzed on rAAV produced following wtAd5 infection of PCL1 in ambr250 bioreactors. 1% DMSO was added at time of cell inoculation into the ambr250 modular bioreactor and wtAd5 infection was performed at 3 hours post cell seeding. Analysis of relative AAV productivity showed a two-fold increase in 1% DMSO treated samples (DMSO was added at the time of ambr250 inoculation, which was 3 hours prior to wtAd5 infection) compared to the no-DMSO control. This result is consistent with data generated in smaller scale infections (FIG. 1A and FIG. 2B). FIG. 4B shows SDS PAGE analysis of VP1:VP2:VP3 ratios in purified vector following detection of the Cap proteins by Coomassie blue staining. An equal number of purified vector genome copies of each sample was loaded onto the SDS-PAGE. The results show that the capsid protein ratios in vector produced in the presence and absence of DMSO are similar. Full capsid percentage (FIG. 4C) as determined by analytical ultracentrifugation (AUC) was slightly (1.5-fold) higher for vector produced in the presence of DMSO. Vector potency was evaluated using an enzyme activity assay for the transgene in PCL1 (FIG. 4D). Cells were transduced with vector purified from control or DMSO-treated samples at MOIs of 1E3 vg/cell and 1E4 vg/cell. Culture supernatants were collected 6 days post-infection and assayed for enzyme activity. Relative enzyme activity as shown in the graph was normalized to the control condition (FIG. 4D). The results show no significant difference in potency between the two vectors produced in the presence and absence of DMSO.

Additional experiments were carried out to understand whether the effect of DMSO on rAAV productivity would extend to transiently transfected mammalian cells. For HEK293 cell transfection, Polyplus FectoVIR was used to transfect a serum-free suspension culture of HEK293 (0.5 μg pDNA per 1E6 viable cells). Two or three plasmid DNAs (e.g., pAdhelper as one plasmid and pAAV-Rep/Cap/ITR/transgene as a second plasmid, or pAdhelper, pAAV-RepCap and pITR-transgene as two separate plasmids) were used for the transfection. The transfection complex was generated using a plasmid DNA:FectoVIR ratio of 1:1 at 5% complexing volume. The transfection complex was then added into HEK293 cells within 25 min of initiation of the complex formation. Either 1% DMSO or an equal volume of culture medium was added at the time of transfection. Transfected cultures were collected at 72 hr post-transfection (hpt) for AAV replication and production analysis. In addition, cell pellets were collected for analysis of KAT5 mRNA via qRT-PCR.

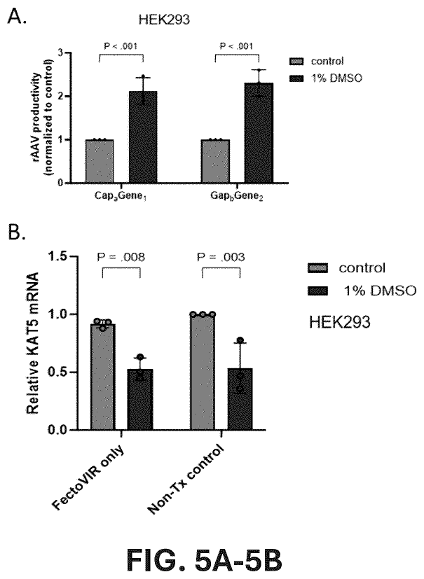

HEK293 cells were transfected to produce two rAAV vectors of different serotypes and transgenes (CapₐGene₁ and Cap_{b}Gene₂) along with transfection reagent only and untransfected controls. Assessment of relative rAAV productivity following DMSO treatment shows about a 2-fold enhancement of rAAV titer with DMSO, regardless of the capsid serotype or transgene. In addition, KAT5 mRNA expression in HEK293 cells treated with 1% DMSO was reduced approximately 2-fold (FIG. 5B). These results suggest that DMSO treatment exerts a positive effect on rAAV production in transiently transfected HEK293 cells as observed in the PCLs. The reduction of KAT5 mRNA expression in DMSO-treated HEK293 cells points toward a possible shared mechanism (see Examples below).

### Example 2. RNA-Seq Analysis Elucidates Regulators of rAAV Production Induced by DMSO

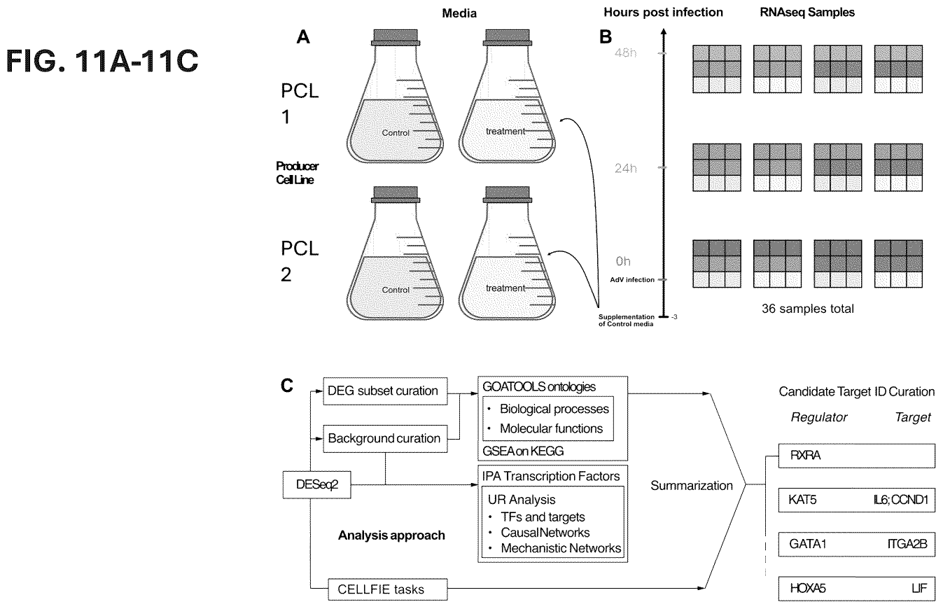

To understand the impact of DMSO on host cell gene expression, infected cell pellets were collected for poly-A enriched RNA-Seq analysis. Briefly, AAV producer cell lines were infected with wtAd5 in the presence or absence of DMSO. For control samples (infection in the absence of DMSO), infection was performed as described above. For DMSO supplemented samples, 1% DMSO were added into growth medium 3 hrs prior to wtAd5 infection (−3 h) or added at the time of infection (0 h) and kept in the medium throughout the infection. Cell pellets were collected and snap frozen at 0 h, 24 h and 48 h post-infection (FIG. 11A-11B). Whole cultures were collected at 48 hpi/72 hpi for AAV titer analysis to confirm the DMSO response. Total RNA extraction, library preparation and Illumina sequencing was performed at Genewiz.

Following the addition of DMSO, there was several fold-increase in AAV transcriptional expression at 48 hours post wtAd5 infection (as described in previous example). Thus, it was clear that the medium supplementation aided in AAV biosynthesis. However, it was unclear what exact changes in cell state favored this increase in AAV expression. Furthermore, the two producer cell lines responded to DMSO to different degrees, and it was unclear how the compositional differences between cell lines could explain the discrepancy in AAV expression. An initial descriptive summary of these changes as induced by DMSO was pursued by a Gene Ontology (GO) Over Representation Analysis (ORA) on the available RNA-Seq data (FIG. 11C).

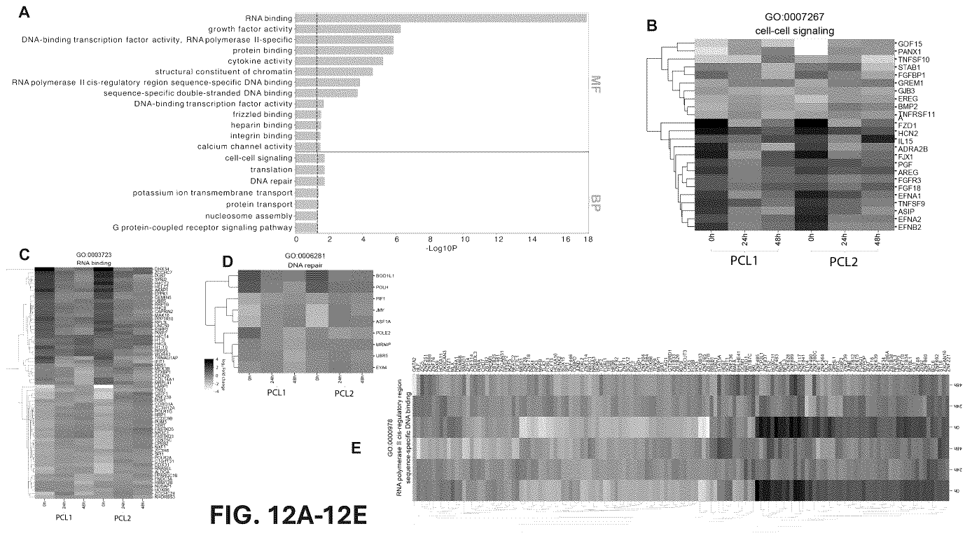

To facilitate this analysis mRNA abundance was quantified in both PCLs to test which GO terms were overrepresented. Specifically, following analysis of differential gene expression in PCL1 in control or supplemented media at each time point, a total of 3327 differentially expressed genes (DEGs) with an absolute log 2 fold-change greater than 1 and a Benjamini Hochberg corrected p-value less than 0.05 were retained for the GO ORA. We found that among a set of curated GO terms, the sets of significantly enriched Biological Process (BP; n=8) and Molecular Function (MF; n=13) GO terms associated with the change in media are supported by literature on AAV PCLs (FIG. 12A). Significantly enriched pathways associated with differentially expressed genes were measured from the comparison of control and DMSO supplemented media in PCL1 and PCL2 and over representation analysis was conducted using GO terms. For example, the top 3 BPs of cell-cell signaling, translation, and DNA repair are all functions known to be targeted directly or through an intermediary by AAV and Ad5 machinery (FIG. 12A).

Upon further analysis, these enriched terms were manually categorized in the context of their biological function, indicated that host transport/localization, translation/transcription, signaling/cell communication, and binding machinery were likely to be affected by DMSO supplementation. Of these, the translation/transcription subsystems retained the greatest number of enriched BP (n=3) and MF (n=6) GO terms with greatest significance (1.306E-18<p<0.043) indicating that supplement-induced changes in upstream transcription factor (TF) expression could be responsible for corresponding increases in rAAV vector titer (Tables 1-4). Correlative support for this observation is drawn from the DE heatmaps of genes as subset by participation in RNA polymerase II cis-regulatory region sequence-specific DNA binding, DNA repair and RNA binding ontologies, where media supplementation of DMSO to PCL1 appears to decrease expression before 24 hours (FIG. 12E). A similar trend is observed for DEGs participating in cell-cell signaling, suggesting an additional role (albeit with unknown impact on viral expression) of media supplementation in signaling and cell communication subsystems (FIG. 12B).

Differentially expressed genes (DEGs) from the comparison of producer cells infected with wtAd5 in the presence or absence of DMSO were used to generate significantly enriched pathways via GO ORA. The FDR corrected p-value cutoff was 0.05 and is represented by vertical dotted black line. GO terms with a p-value<0.05 were considered as significant (FIG. 12A). DEGs were shown as log 2 Fold Change in heatmaps for example GO terms (FIG. 12B). FIG. 12C is a table of transcription factors and unique targets associated with an increase in viral titer predicted by Ingenuity Pathway Analysis (IPA).

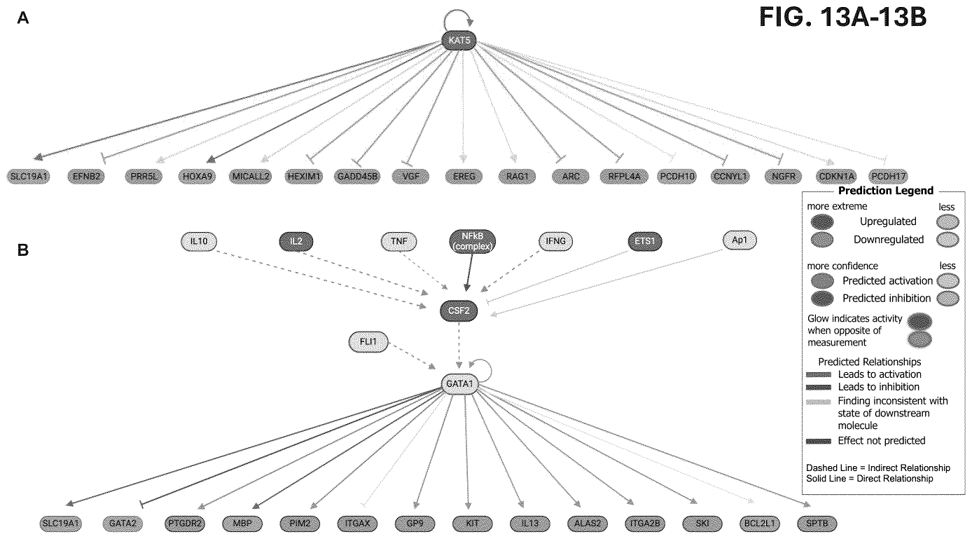

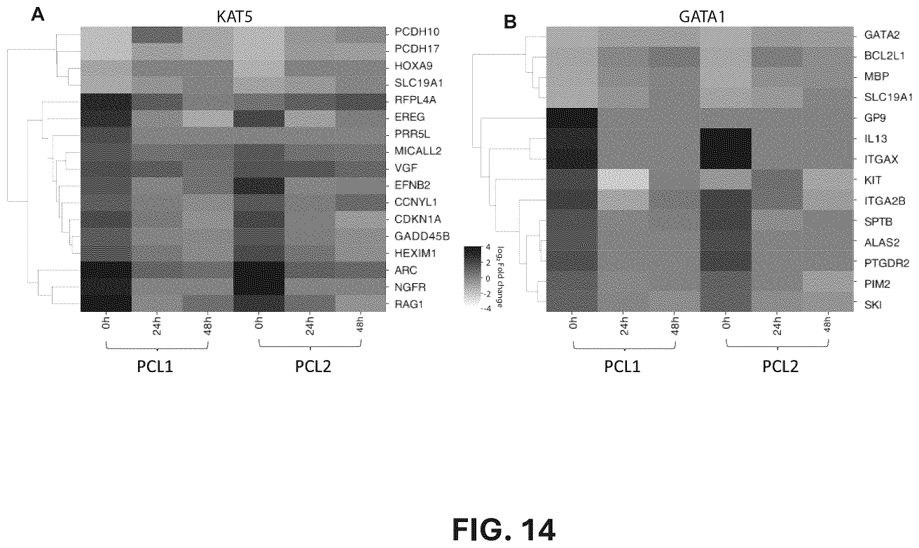

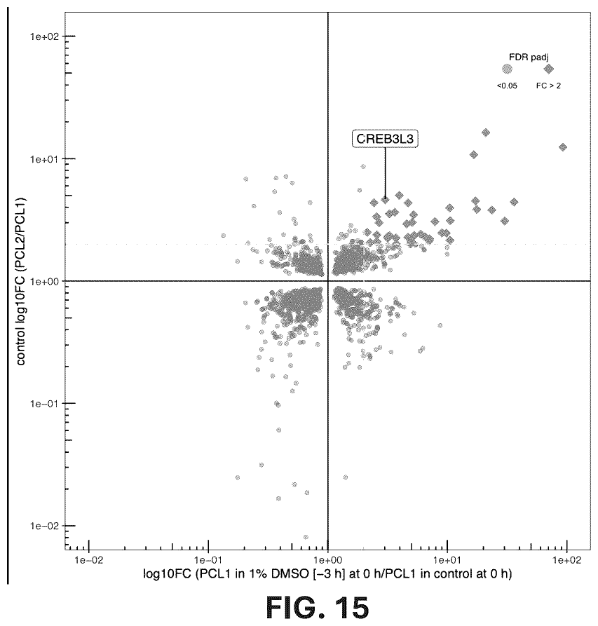

To identify potential cell engineering targets, GO ORA results were leveraged, along with an analysis of upstream regulators of differential gene expression induced by DMSO. The rationale for this was the hypothesis that upstream transcription factors (TFs) of differentially expressed genes could be responsible for the corresponding increases in AAV viral expression observed following DMSO treatment. Emulation of the rAAV titer response by knock down (KD), knock out (KO), or overexpression of participating TFs can facilitate both a deeper understanding of the mechanistic networks driving rAAV biosynthesis and can eliminate the costs associated with DMSO supplementation. Several TFs and their targets were identified using IPA through use of the mechanistic and causal network features in the upstream regulator analysis (URA). Multiple candidate TFs and corresponding targets unique to each TF were identified as potential upstream regulators of overall rAAV viral expression (Tables 1-4; FIG. 13-14). Further quantitative information on each identified upstream regulator UR and their comprehensive set of downstream targets as identified from IPA URA that pass both mechanistic and causal network filters are listed in Table 3 and Table 4. Additional upstream regulators and targets of interest identified solely from the URA include CCND1, SS18, HDAC5, SMAD4, NME1, PTEN, BCL2, las1L, TGM2, NGEF, TRPC1, HHEX, LIF, IL6, FAS, ITGA2B, NFKBIA, HSP90B1, TCF7, RIGI, FGFR3, ADM2, and NFKB1 and can be found in Tables 1-4. Finally, additional genes of interest with known response to viral infection identified solely from differential expression testing analyses were identified. Brief descriptions of example candidate TFs are as follows:

Chromodomain-Helicase DNA-binding 1 (CHD1) is a chromatin remodeling protein that is highly conserved across species and is named after the functional capacity of their chromo, helicase-like and DNA-binding domains that bind to lysine residues, nucleic acids, and methylated histone H3K4 typically associated with the 5′ end of actively transcribed genes.

Evolutionarily conserved signaling intermediate in toll pathway (ECSIT) is a protein with at least 2 functional isozymes participating in viral infection pathology, alongside typical cellular physiological processes of redox metabolism, innate immunity, and development.

Forkhead box like 2 (FOXL2) is a member of the functionally diverse FOX family of TFs, all of which are identifiable by a 100-residue forkhead DNA binding domain; FOXL2 functional capacity is pivotal for ovarian development.

Homeobox A5 (HOXA5) is a member of the HOX family of TFs associated with pivotal roles in embryonic development; HOXA5 is positively regulated by another TF, Yin Yang 1 (YY1) and its expression can upregulate regulatory tumor protein p53 expression.

Retinoid X receptor alpha (RXRα), in response to retinoic acid (RA) as ligand, participates in either homo- or hetero-dimer complexes with RA receptors to bind the RA response element of target gene promoters in order to regulate their expression.

Thioesterase superfamily member 6 (THEM6) is a member of the ER-associated THEs participating in the regulation of fatty acid intracellular trafficking, lipogenesis and β-oxidation; overexpression of THEM6 has been shown to increase de novo sterol synthesis. GATA-binding factor 1 (GATA1) is one of 6 members within the family so called for the recognition and binding of the DNA consensus sequence GATA and maintains essential functions in both normal erythropoiesis and regulation of the G1/S cell cycle progression. FIG. 14B shows GATA1 downstream target gene expression as influenced by Ad5 infection over time.

Histone acetyltransferase KAT5 is a member of the MYST family of acetyltransferases that can acetylate histones in the nucleosome allowing for the interaction of other TFs. FIG. 14A shows KAT5 downstream target gene expression as influenced by Ad5 infection over time.

### Example 3. SiRNA Knockdown and AAV Productivity Impact Assessment

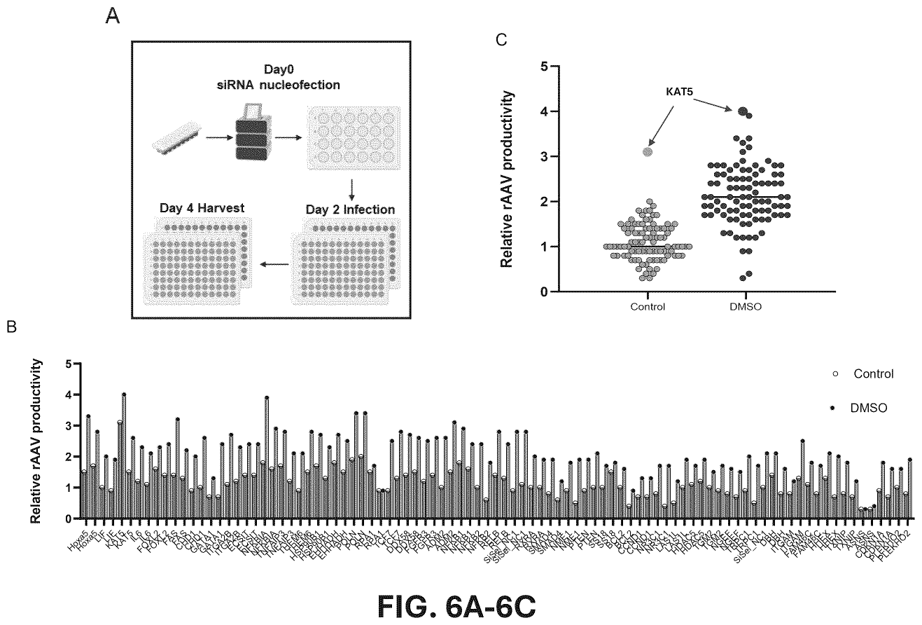

To assess the impact of target gene knockdown on AAV production in the PCLs, cells were transfected with siRNA followed by wtAd5 infection in the presence or absence of 1% DMSO treatment at the time of infection (schematic of experimental design provided in FIG. 6A). siRNAs against several target genes of interest identified from the RNA-Seq analysis were resuspended with nuclease-free sterile water to create a 10 μM stock solution (Table 5). 20 μmol siRNA against each target were transfected into 4E5 total viable producer cells in Nucleocuvette Strip format using Lonza 4D nucleofector and SE Cell Line 4D-Nucleofector X kit S (Lonza) following the manufacturer's protocol. Cells were resuspended and cultured in 24-well plates for 48 hours before infection with wtAd5 and then harvested for assessment of AAV production at 48 hpi (corresponds to 96 hours post-transfection (hpt) with the siRNA). The results of the siRNA screen (bar graph, FIG. 6B; scatterplot, FIG. 6C) identify KAT5 as a promising target. Knockdown of KAT5 increases rAAV productivity about 2-fold while no further increase is observed following addition of DMSO. These results suggest that KAT5 might be involved in the mechanism of action of DMSO.

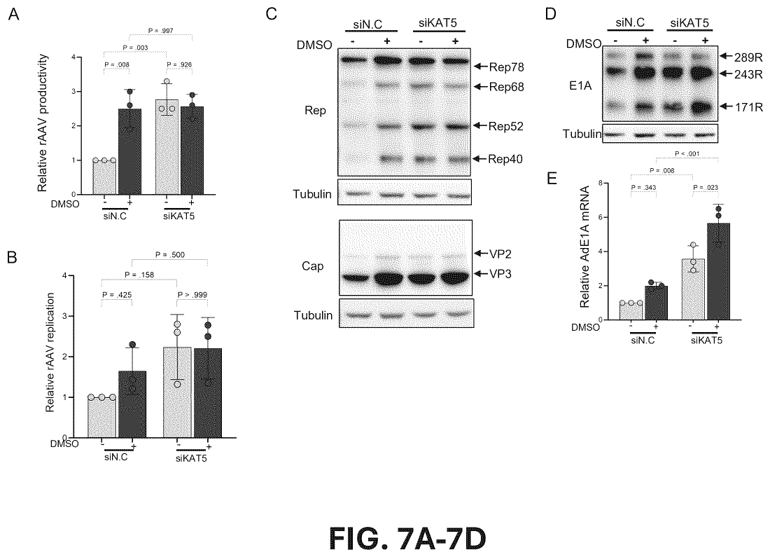

PCL1 was transfected with either a negative control siRNA (siN.C) or siKAT5 followed by Ad5 infection as described above using the Lonza 4D nucleocuvette with 4E6 total viable cells. Cell cultures were collected for evaluation of rAAV DNA replication and rAAV production; cell pellets were collected for gene expression analysis via RT-qPCR and assessment of protein expression via Western Blot. rAAV productivity was evaluated following knockdown of the KAT5 gene (FIG. 7A). Values shown in the graph were normalized to the PCL1 transfection with siN.C in the absence of DMSO. In cells transfected with siN.C, supplementation with 1% DMSO increased rAAV productivity approximately 2.5-fold. Cells transfected with siKAT5 showed a similar 2.5-fold increase in rAAV production in both the absence or presence of DMSO. rAAV DNA replication was assessed similarly (FIG. 7B). Replication was not significantly increased with DMSO treatment in control cells or with knockdown of KAT5. The results suggest that knockdown of KAT5 simulates the effect of DMSO on rAAV production in PCL1.

Expression of the AAV Rep and Cap (FIG. 7C) and Adenoviral E1A (FIG. 7D) proteins were evaluated by Western blot. In cells transfected with the control siRNA, an increase in expression of all four AAV Rep proteins (Rep78, 68, 52 and 40) as well as the AAV Cap proteins was observed with DMSO treatment. Knockdown of KAT5 also increases expression of AAV Rep and Cap proteins relative to the control (siN.C, -DMSO) both in the presence and absence of DMSO. Likewise, AdE1A protein expression is enhanced by DMSO treatment in control cells and by KAT5 knockdown independent of DMSO treatment (FIG. 7D). Overall, the analysis of AAV Rep, Cap and AdE1A expression via Western suggests that KAT5 knockdown recapitulates the effect of DMSO on expression of these viral proteins.

To assess whether the increase in AdE1A protein expression results from enhanced E1A gene transcription, mRNA levels were analyzed via qRT-PCR (FIG. 7E). Values shown in the graph were normalized to cells transfected with siN.C in the absence of DMSO. Though a trend toward an increase was observed, AdE1A mRNA was not significantly increased in control cells treated with DMSO, while in cells transfected with siKAT5, AdE1A mRNA was increased approximately 3.5-fold and 6-fold in the absence and presence of DMSO, respectively.

TABLE 5
siRNA Target List

| | RefSeq | | | | |
|---|---|---|---|---|---|
| | Accession | Gene | | | siRNA |
| Numbers | Number | Symbol | Full Gene Name | Gene ID | ID |
| 1 | NM_024866 | ADM2 | adrenomedullin 2 | 79924 | s36652 |
| 2 | NM_024866 | ADM2 | adrenomedullin 2 | 79924 | s36653 |
| 3 | NM_000657 | BCL2 | B-cell CLL/lymphoma 2 | 596 | s1915 |
| 4 | NM_000657 | BCL2 | B-cell CLL/lymphoma 2 | 596 | s1916 |
| 5 | NM_053056 | CCND1 | cyclin D1 | 595 | s229 |
| 6 | NM_053056 | CCND1 | cyclin D1 | 595 | s230 |
| 7 | NM_001270 | CHD1 | chromodomain helicase | 1105 | s2974 |
| | | | DNA binding protein 1 | | |
| 8 | NM_001270 | CHD1 | chromodomain helicase | 1105 | s2975 |
| | | | DNA binding protein 1 | | |
| 9 | NM_014314 | DDX58 | DEAD (Asp-Glu-Ala- | 23586 | s24143 |
| | | | Asp) box polypeptide 58 | | |
| 10 | NM_014314 | DDX58 | DEAD (Asp-Glu-Ala- | 23586 | s24144 |
| | | | Asp) box polypeptide 58 | | |
| 11 | NM_016581 | ECSIT | ECSIT homolog | 51295 | s27873 |
| | | | (Drosophila) | | |
| 12 | NM_016581 | ECSIT | ECSIT homolog | 51295 | s27874 |
| | | | (Drosophila) | | |
| 13 | NM_001966 | EHHADH | enoyl-Coenzyme A, | 1962 | s4549 |
| | | | hydratase/3-hydroxyacyl | | |
| | | | Coenzyme A | | |
| | | | dehydrogenase | | |
| 14 | NM_001966 | EHHADH | enoyl-Coenzyme A, | 1962 | s4550 |
| | | | hydratase/3-hydroxyacyl | | |
| | | | Coenzyme A | | |
| | | | dehydrogenase | | |
| 15 | NM_000043 | FAS | Fas (TNF receptor | 355 | s1506 |
| | | | superfamily, member 6) | | |
| 16 | NM_000043 | FAS | Fas (TNF receptor | 355 | s1507 |
| | | | superfamily, member 6) | | |
| 17 | NM_000142 | FGFR3 | fibroblast growth factor | 2261 | s5167 |
| | | | receptor 3 | | |
| 18 | NM_000142 | FGFR3 | fibroblast growth factor | 2261 | s5168 |
| | | | receptor 3 | | |
| 19 | XM_001131060 | FOXL2 | forkhead box L2 | 668 | s2068 |
| 20 | XM_001131060 | FOXL2 | forkhead box L2 | 668 | s2069 |
| 21 | NM_002049 | GATA1 | GATA binding protein 1 | 2623 | s5593 |
| | | | (globin transcription | | |
| | | | factor 1) | | |
| 22 | NM_002049 | GATA1 | GATA binding protein 1 | 2623 | s5594 |
| | | | (globin transcription | | |
| | | | factor 1) | | |
| 23 | NM_005474 | HDAC5 | histone deacetylase 5 | 10014 | s19462 |
| 24 | NM_005474 | HDAC5 | histone deacetylase 5 | 10014 | s19463 |
| 25 | NM_010453 | Hoxa5 | homeobox A5 | 15402 | s67675 |
| 26 | NM_010453 | Hoxa5 | homeobox A5 | 15402 | s67676 |
| 27 | NM_003299 | HSP90B1 | heat shock protein | 7184 | s14373 |
| | | | 90 kDa beta (Grp94), | | |
| | | | member 1 | | |
| 28 | NM_003299 | HSP90B1 | heat shock protein | 7184 | s14374 |
| | | | 90 kDa beta (Grp94), | | |
| | | | member 1 | | |
| 29 | NM_000600 | IL6 | interleukin 6 | 3569 | s7311 |
| | | | (interferon, beta 2) | | |
| 30 | NM_000600 | IL6 | interleukin 6 | 3569 | s7312 |
| | | | (interferon, beta 2) | | |
| 31 | NM_000419 | ITGA2B | integrin, alpha 2b | 3674 | s7538 |
| | | | (platelet glycoprotein | | |
| | | | IIb of IIb/IIIa | | |
| | | | complex, antigen | | |
| | | | CD41) | | |
| 32 | NM_000419 | ITGA2B | integrin, alpha 2b | 3674 | s7539 |
| | | | (platelet glycoprotein | | |
| | | | IIb of IIb/IIIa | | |
| | | | complex, antigen | | |
| | | | CD41) | | |
| 33 | NM_006388 | KAT5 | K(lysine) | 10524 | s20629 |
| | | | acetyltransferase 5 | | |
| 34 | NM_006388 | KAT5 | K(lysine) | 10524 | s20630 |
| | | | acetyltransferase 5 | | |
| 35 | NM_031206 | LAS1L | LAS1-like (S. cerevisiae) | 81887 | s37860 |
| 36 | NM_031206 | LAS1L | LAS1-like (S. cerevisiae) | 81887 | s37861 |
| 37 | NM_002309 | LIF | leukemia inhibitory | 3976 | s8168 |
| | | | factor (cholinergic | | |
| | | | differentiation | | |
| | | | factor) | | |
| 38 | NM_002309 | LIF | leukemia inhibitory | 3976 | s8169 |
| | | | factor (cholinergic | | |
| | | | differentiation | | |
| | | | factor) | | |
| 39 | NM_003998 | NFKB1 | nuclear factor | 4790 | s9504 |
| | | | of kappa light | | |
| | | | polypeptide gene | | |
| | | | enhancer in | | |
| | | | B-cells 1 | | |
| 40 | NM_003998 | NFKB1 | nuclear factor | 4790 | s9505 |
| | | | of kappa light | | |
| | | | polypeptide gene | | |
| | | | enhancer in | | |
| | | | B-cells 1 | | |
| 41 | NM_001077493 | NFKB2 | nuclear factor | 4791 | s9507 |
| | | | of kappa light | | |
| | | | polypeptide gene | | |
| | | | enhancer in | | |
| | | | B-cells 2 | | |
| | | | (p49/p100) | | |
| 42 | NM_001077493 | NFKB2 | nuclear factor | 4791 | s9508 |
| | | | of kappa light | | |
| | | | polypeptide gene | | |
| | | | enhancer in | | |
| | | | B-cells 2 | | |
| | | | (p49/p100) | | |
| 43 | NM_020529 | NFKBIA | nuclear factor | 4792 | s9510 |
| | | | of kappa light | | |
| | | | polypeptide gene | | |
| | | | enhancer in | | |
| | | | B-cells | | |
| | | | inhibitor, | | |
| | | | alpha | | |
| 44 | NM_020529 | NFKBIA | nuclear factor | 4792 | s9511 |
| | | | of kappa light | | |
| | | | polypeptide | | |
| | | | gene enhancer | | |
| | | | in B-cells | | |
| | | | inhibitor, | | |
| | | | alpha | | |
| 45 | NM_019850 | NGEF | neuronal | 25791 | s24487 |
| | | | guanine | | |
| | | | nucleotide | | |
| | | | exchange | | |
| | | | factor | | |
| 46 | NM_019850 | NGEF | neuronal | 25791 | s230253 |
| | | | guanine | | |
| | | | nucleotide | | |
| | | | exchange | | |
| | | | factor | | |
| 47 | NM_198175 | NME1 | NME/NM23 | 4830 | s9588 |
| | | | nucleoside | | |
| | | | diphosphate | | |
| | | | kinase 1 | | |
| 48 | NM_198175 | NME1 | NME/NM23 | 4830 | s9590 |
| | | | nucleoside | | |
| | | | diphosphate | | |
| | | | kinase 1 | | |
| 49 | NM_001024094 | NR3C1 | nuclear | 2908 | s6186 |
| | | | receptor | | |
| | | | subfamily | | |
| | | | 3, group C, | | |
| | | | member 1 | | |
| | | | (glucocorticoid | | |
| | | | receptor) | | |
| 50 | NM_001024094 | NR3C1 | nuclear | 2908 | s6187 |
| | | | receptor | | |
| | | | subfamily | | |
| | | | 3, group C, | | |
| | | | member 1 | | |
| | | | (glucocorticoid | | |
| | | | receptor) | | |
| 51 | NM_181808 | POLN | polymerase | 353497 | s51479 |
| | | | (DNA directed) | | |
| | | | nu | | |
| 52 | NM_181808 | POLN | polymerase | 353497 | s51480 |
| | | | (DNA directed) | | |
| | | | nu | | |
| 53 | NM_000314 | PTEN | phosphatase and | 5728 | s325 |
| | | | tensin homolog | | |
| 54 | NM_000314 | PTEN | phosphatase and | 5728 | s326 |
| | | | tensin homolog | | |
| 55 | NM_006509 | RELB | v-rel | 5971 | s11917 |
| | | | reticuloendotheliosis | | |
| | | | viral oncogene | | |
| | | | homolog B | | |
| 56 | NM_006509 | RELB | v-rel | 5971 | s11918 |
| | | | reticuloendotheliosis | | |
| | | | viral oncogene | | |
| | | | homolog B | | |
| 57 | NM_002945 | RPA1 | replication | 6117 | s12127 |
| | | | protein A1, 70 | | |
| | | | kDa | | |
| 58 | NM_002945 | RPA1 | replication | 6117 | s12128 |
| | | | protein A1, 70 | | |
| | | | kDa | | |
| 59 | NM_002957 | RXRA | retinoid X receptor, alpha | 6256 | s12384 |
| 60 | NM_002957 | RXRA | retinoid X receptor, alpha | 6256 | s12385 |
| 61 | NM_005359 | SMAD4 | SMAD family member 4 | 4089 | s8403 |
| 62 | NM_005359 | SMAD4 | SMAD family member 4 | 4089 | s8405 |
| 63 | NM_005637 | SS18 | synovial sarcoma | 6760 | s13510 |
| | | | translocation, | | |
| | | | chromosome 18 | | |
| 64 | NM_005637 | SS18 | synovial sarcoma | 6760 | s13511 |
| | | | translocation, | | |
| | | | chromosome 18 | | |
| 65 | NM_003202 | TCF7 | transcription factor 7 (T- | 6932 | s13877 |
| | | | cell specific, HMG-box) | | |
| 66 | NM_003202 | TCF7 | transcription factor 7 (T- | 6932 | s13878 |
| | | | cell specific, HMG-box) | | |
| 67 | NM_198951 | TGM2 | transglutaminase 2 | 7052 | s14087 |
| 68 | NM_198951 | TGM2 | transglutaminase 2 | 7052 | s14088 |
| 69 | NM_016647 | THEM6 | thioesterase superfamily | 51337 | s27976 |
| | | | member 6 | | |
| 70 | NM_016647 | THEM6 | thioesterase superfamily | 51337 | s27977 |
| | | | member 6 | | |
| 71 | NM_006290 | TNFAIP3 | tumor necrosis factor, | 7128 | s14259 |
| | | | alpha-induced protein 3 | | |
| 72 | NM_006290 | TNFAIP3 | tumor necrosis factor, | 7128 | s14260 |
| | | | alpha-induced protein 3 | | |
| 73 | NM_003304 | TRPC1 | transient receptor | 7220 | s14409 |
| | | | potential cation channel, | | |
| | | | subfamily C, member 1 | | |
| 74 | NM_003304 | TRPC1 | transient receptor | 7220 | $14410 |
| | | | potential cation channel, | | |
| | | | subfamily C, member 1 | | |
| 76 | NM_000787 | DBH | dopamine beta- | 1621 | s3946 |
| | | | hydroxylase (dopamine | | |
| | | | beta-monooxygenase) | | |
| 77 | NM_000632 | ITGAM | integrin, alpha M | 3684 | s7565 |
| | | | (complement component | | |
| | | | 3 receptor 3 subunit) | | |
| 78 | NM_000632 | ITGAM | integrin, alpha M | 3684 | s7566 |
| | | | (complement component | | |
| | | | 3 receptor 3 subunit) | | |
| 79 | NM_017709 | FAM46C | family with sequence | 54855 | s29596 |
| | | | similarity 46, member C | | |
| 80 | NM_017709 | FAM46C | family with sequence | 54855 | s29597 |
| | | | similarity 46, member C | | |
| 81 | NM_002729 | HHEX | hematopoietically | 3087 | s6534 |
| | | | expressed homeobox | | |
| 82 | NM_002729 | HHEX | hematopoietically | 3087 | s6535 |
| | | | expressed homeobox | | |
| 83 | NM_006472 | TXNIP | thioredoxin interacting | 10628 | s20878 |
| | | | protein | | |
| 84 | NM_006472 | TXNIP | thioredoxin interacting | 10628 | s20879 |
| | | | protein | | |
| 85 | NM_133436 | ASNS | asparagine synthetase | 440 | s1678 |
| 86 | NM_133436 | ASNS | asparagine synthetase | 440 | s1679 |
| 87 | NM_078467 | CDKN1A | cyclin-dependent kinase | 1026 | s415 |
| | | | inhibitor 1A (p21, Cip1) | | |
| 88 | NM_078467 | CDKN1A | cyclin-dependent kinase | 1026 | s416 |
| | | | inhibitor 1A (p21, Cip1) | | |
| 89 | NM_025201 | PLEKHO2 | pleckstrin homology | 80301 | s37165 |
| | | | domain containing, family | | |
| | | | O member 2 | | |
| 90 | NM_025201 | PLEKHO2 | pleckstrin homology | 80301 | s37166 |
| | | | domain containing, family | | |
| | | | O member 2 | | |
| 91 | NM_173667 | C5ORF64 | hypothetical protein | 285668 | s49998 |
| | | | FLJ37543 | | |
| 92 | NM_173667 | C5ORF64 | hypothetical protein | 285668 | s49999 |
| | | | FLJ37543 | | |
| 93 | NM_020882 | COL20A1 | collagen, type XX, alpha | 57642 | s33496 |
| | | | 1 | | |
| 94 | NM_020882 | COL20A1 | collagen, type XX, alpha | 57642 | s33497 |
| | | | 1 | | |
| 95 | NM_024009 | GJB3 | gap junction protein, beta | 2707 | s5778 |
| | | | 3, 31 kDa | | |
| 96 | NM_024009 | GJB3 | gap junction protein, beta | 2707 | s5779 |
| | | | 3, 31 kDa | | |
| 97 | NM_016368 | ISYNA1 | inositol-3-phosphate | 51477 | s28195 |
| | | | synthase 1 | | |
| 98 | NM_016368 | ISYNA1 | inositol-3-phosphate | 51477 | s28196 |
| | | | synthase 1 | | |
| 99 | NR_028339 | PARD6G- | PARD6G antisense RNA | 100130522 | n514828 |
| | | AS1 | 1 | | |
| 100 | NR_028339 | PARD6G- | PARD6G antisense RNA | 100130522 | n514829 |
| | | AS1 | 1 | | |

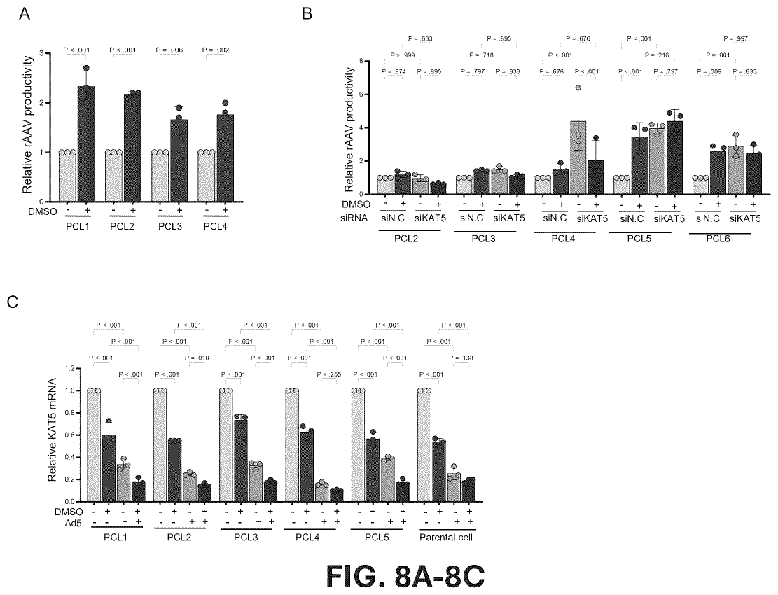

The universality of the DMSO treatment effect and KAT5 knockdown was assessed in additional cell lines (PCL1-6) which produce five different vectors composed of four different capsids and expressing five transgenes. Relative rAAV productivity was examined in PCL1, PCL2, PCL3, and PCL4, in deep well plates with or without (+/−) 1% DMSO treatment at the time of infection (0 h) (FIG. 8A). The results show that DMSO enhances rAAV productivity 2-3 fold in all four PCLs in the deep well culture condition, including PCL2 which showed no AAV productivity response to DMSO in shake flasks in three independent runs shown in FIG. 2B.

KAT5 knockdown was evaluated in five different PCLs (FIG. 8B) as previously described in FIG. 6A. Relative rAAV productivity was analyzed following transfection with KAT5 siRNA (s20629) or scramble control siRNA (siN.C) with (+) or without (−) DMSO supplementation and normalized to transfection with the control siRNA in the absence of DMSO treatment. siKAT5 knockdown increased AAV production from PCL4, PCL5, and PCL6 in the absence of DMSO, but not from PCL2 and PCL3, which differs from the observations made upon DMSO treatment in deep well plates as shown in FIG. 8A. However, this was consistent with the results shown in FIG. 2B, showing no effect of DMSO on PCL2 in three independent shake flask tests. In PCL5 and PCL6, siKAT5 or DMSO treatment alone each increased AAV production to a similar level but DMSO treatment in addition to KAT5 knockdown did not lead to a further increase in productivity. In contrast, knockdown of KAT5 in PCL4 in the absence of DMSO increased productivity 4-fold while no significant effect was observed following DMSO treatment. These results suggest DMSO increases AAV production partly via down-regulation of KAT5 at least from PCL1, PCL5 and PCL6.

To further evaluate if the downregulation of KAT5 mRNA expression is a general cellular response to DMSO treatment, five different PCLs as well as the parental host cell were cultured in spin tubes with or without 1% DMSO and with or without wtAd5 infection. Cell pellet samples were collected at 48 hours post cell seeding for KAT5 mRNA analysis using RT-qPCR (FIG. 8C). Total RNA was extracted using the RNeasy plus kit (Qiagen) according to manufacturer's instructions, and reverse transcribed to cDNA using Applied Biosystems™ High-Capacity cDNA Reverse Transcription Kit (Applied Biosystems) followed by qPCR with QuantStudio7 flex using primers and probes listed in Table 6. Relative KAT5 mRNA levels shown in the bar graph were calculated following normalization to the uninfected, non-treated control condition for each cell line. The results (FIG. 8C) show that in the absence of DMSO, wtAd5 infection reduces KAT5 mRNA levels in each cell line, with the magnitude ranging from 2.5 to 5-fold. In uninfected cells, KAT5 mRNA is decreased approximately 2-fold by DMSO treatment. In wtAd5-infected cells, DMSO exerts an additive effect and further suppresses KAT5 mRNA levels in most cell lines (exceptions are PCL4 and the parental cells).

TABLE 6
Primers/probe sequences

| Primer, probes | Oligo nucleotides | Source | Identifier |
|---|---|---|---|
| BGH-Froward primer | TCTAGTTGCCAGCCATCTGTTGT | Integrated DNA | |
| | (SEQ ID NO: 1) | Technologies | |
| BGH-Reverse primer | TGGGAGTGGCACCTTCCA | Integrated DNA | |
| | (SEQ ID NO: 2) | Technologies | |
| BGH-Probe | TCCCCCGTGCCTTCCTTGACC | Integrated DNA | |
| | (SEQ ID NO: 3) | Technologies | |
| E1A-Forward primer | GTCCTTCTAACACACCTCCTG | Integrated DNA | |
| | (SEQ ID NO: 4) | Technologies | |
| E1A-Reverse primer | CAGGCTGTGGAATGTATCGAG | Integrated DNA | |
| | (SEQ ID NO: 5) | Technologies | |
| E1A-Probe | TGTGCCCCATTAAACCAGTTGCC | Integrated DNA | |
| | (SEQ ID NO: 6) | Technologies | |
| E2A-Forward primer | TTGCGTCGGTGTTGGAGAT | Integrated DNA | |
| | (SEQ ID NO: 7) | Technologies | |
| E2A-Reverse primer | CAAGGCCAAGATCGTGAAGAA | Integrated DNA | |
| | (SEQ ID NO: 8) | Technologies | |
| E2A-Probe | TGCACCACATTTCGGCCCCA | Integrated DNA | |
| | (SEQ ID NO: 9) | Technologies | |
| HPRTI primer/probe | | Invitrogen | Hs99999909 |
| 20x mix | | | _ml |
| IL-6 primer/probe | | Invitrogen | Hs00174131 |
| 20x mix | | | _ml |
| CCND1 primer/probe | | Invitrogen | Hs00765553 |
| 20x mix | | | _ml |
| KAT5 primer/probe | | Invitrogen | Hs00197310 |
| 20x mix | | | _ml |

### Example 4. Cytokine Expression Analysis

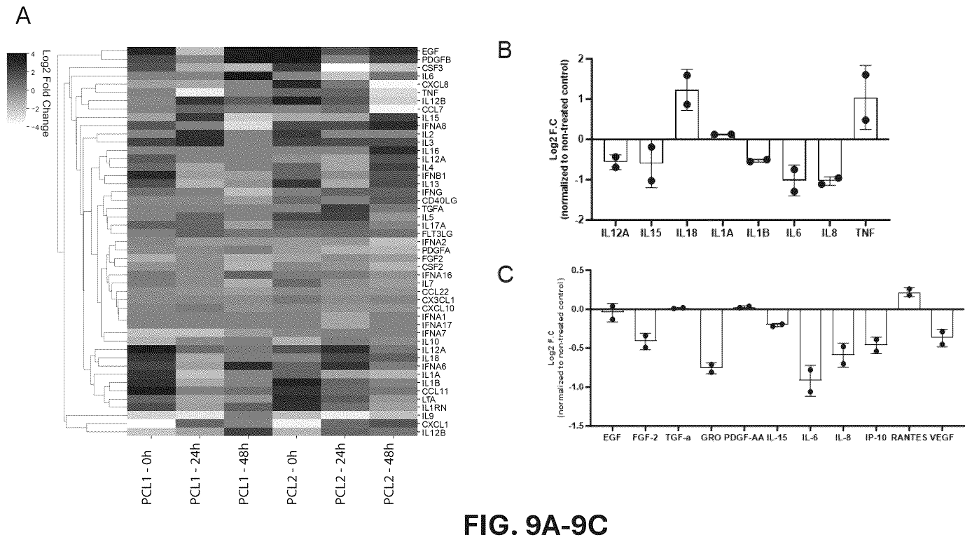

DMSO has been shown to affect the production of pro-inflammatory cytokines; therefore, cytokine expression was evaluated to determine whether the impact of DMSO on rAAV production might be mediated by cytokine production. Differential expression of cytokine genes (FIG. 9A) in PCL1 and PCL2 were derived via RNA-Seq analysis. RNA-Seq analysis is described in Example 2.

Cell pellet samples collected at 24 hpi from the control (no DMSO) and DMSO-treated cultures shown in FIGS. 2A-2G were used for cytokine array analysis. Total RNA extraction was performed using the Qiagen RNAeasy kit. Reverse transcription was performed to generate cDNA (Applied Biosystems), and subjected to TaqMan cytokine array analysis (Applied Biosystems) following the manufacturer's protocol. Array results are shown in FIG. 9B for cytokines with a Ct value less than 30. Values shown are fold change normalized to both a housekeeping gene (HPRT1) and the non-treated control.

For analysis of cytokine secretion, culture supernatants were collected at 0 and 24 hpi from the control (no DMSO) and DMSO-treated wtAd5-infected PCL1 cultures. Luminex assays were performed using multiplex human cytokine/chemokine magnetic bead panel (Millipore) following the manufacturer's protocol by DC3 Therapeutics. Luminex assay results shown in FIG. 9C were for cytokines with values above the limit of quantification (LOQ).

DMSO treatment led to broad changes in cytokine gene expression as depicted in the RNAseq analysis heatmap (FIG. 9A). DMSO treatment reduced mRNA expression of multiple cytokines including IL-6, IL-8, IL-15, IL-12A, IL-1A, IL-1B but expression of IL-18 and TNF were increased. Reductions in IL-6, IL-8 and IL-15 were confirmed by both the cytokine array (FIG. 9B) and Luminex (FIG. 9C) analyses.

### Example 5. Knockdown of KAT5 Reduced CCND1 Expression while not Altering IL-6 Expression

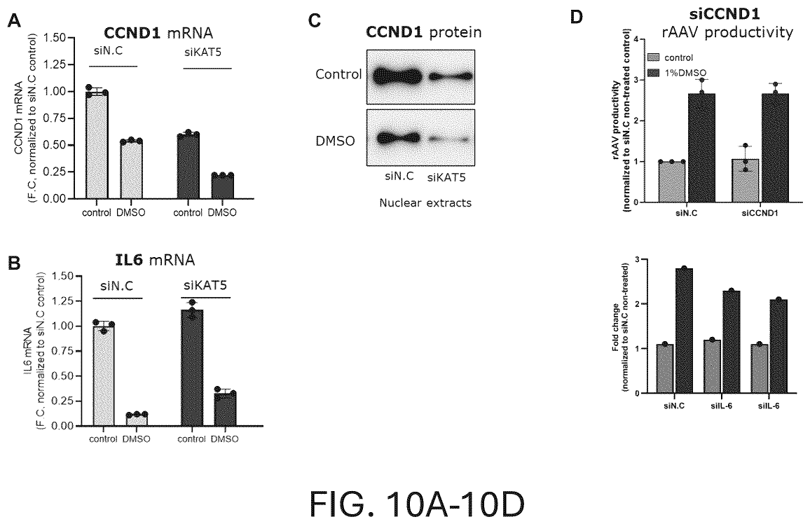

As a histone acetyltransferase, KAT5 may influence the expression of multiple downstream genes. Expression of CCDN1 and IL-6, two genes known to be regulated by KAT5 was assessed in PCL1 following KAT5 gene knockdown in the presence or absence of DMSO. CCND1 and IL-6 mRNA levels were analyzed via qRT-PCR and presented as fold change normalized to the negative control (transfection with siN.C, no DMSO). CCND1 mRNA (FIG. 10A) was reduced two-fold (to 50% of the negative control) following DMSO treatment of cells transfected with the control siRNA (siN.C). In cells transfected with siKAT5, a similar two-fold reduction (to 50%) compared to the negative control was observed in the absence of DMSO while treatment with DMSO reduced CCND1 mRNA an additional two-fold (to ˜25% of the control). CCND1 protein expression as analyzed by Western blot (FIG. 10C) was consistent with the mRNA analysis, showing a reduction with both DMSO and KAT5 knockdown. Overall, these data suggest that both KAT5 knockdown and DMSO reduce the expression of CCND1 and that an additive effect in observed in cells subjected to both treatments.

Though KAT5 knockdown reduced CCND1 mRNA levels, IL-6 mRNA abundance was not affected by KAT5 knockdown (FIG. 10B). Cells transfected with siN.C and siKAT5 showed similar levels of 1L-6 mRNA; however, treatment with DMSO reduced IL-6 mRNA in both cases (˜8 and ˜4-fold, respectively). This result is consistent with the cytokine expression analysis (Example 4). Therefore, expression of IL-6, though reduced by DMSO, is not influenced by KAT5.

To directly assess the effect of reduced CCND1 and IL-6 expression on rAAV production, the genes were knocked down via transfection of PCL1 with siRNA's targeting each gene as previously described in FIG. 6A. rAAV productivity was not changed compared to the control (transfection with siN.C, no DMSO) following knockdown of CCND1 (FIG. 10C). In contrast, rAAV production was increased ˜2.5-fold by DMSO treatment in cells transfected with the control siRNA and siCCND1. A similar result was observed following knockdown of IL-6 (FIG. 10D).

Overall, the results of these experiments show that DMSO treatment reduces the expression of both CCND1 and IL-6 in PCL1; however, KAT5 influences the expression of CCND1 only. Knockdown of CCND1 or IL-6 did not affect rAAV production, therefore, the effect of KAT5 knockdown on rAAV production is likely not mediated by either CCND1 or IL-6 in the PCLs tested. Notably, KAT5 knockdown increases expression of AdE1A protein (see Example 3). This observation is consistent with a report in the literature that the product of the KAT5 gene, TIP60, represses expression of AdE1A (Gupta et al, Oncogene, 2013). Since AdE1A activates transcription of adenoviral genes as well as the AAV p5 promoter which drives transcription of the rep gene, the effect of KAT5 knockdown on AdE1A expression may explain in part how DMSO (and knockdown of KAT5) enhances rAAV production.

### Example 6. AAV Production from Knockdown of Select Additional Genes

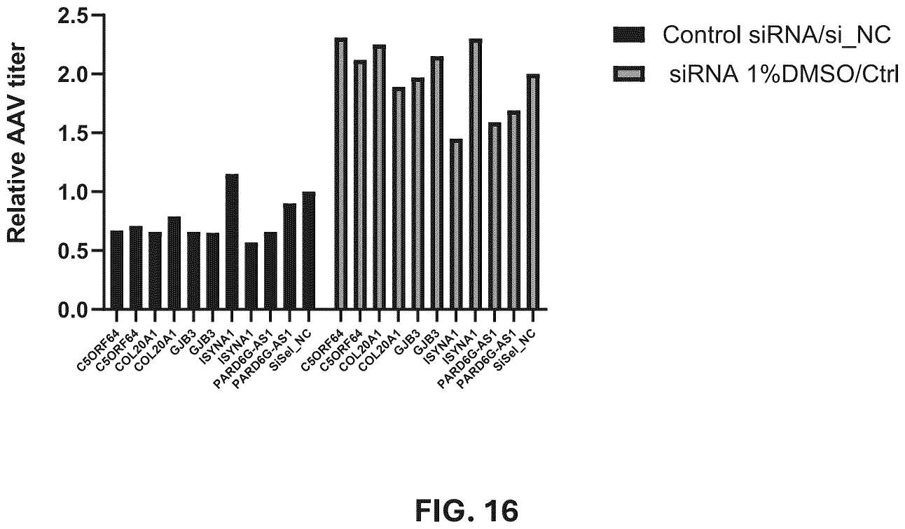

An siRNA screen in PCL1 was performed for additional target genes identified in the RNA-Seq experiments. Two siRNA candidates for each selected target or negative control were transfected into PCL1 cells using Lonza nucleofector 4D. Transfected cells were cultured for 2 days before infecting with wtAd5 with or without the presence of 1% DMSO. rAAV titer was analyzed to assess the impact of target knockdown with samples collected at 2 days post infection. Productivity from control infection condition without DMSO from each target was normalized to that of siNC. Productivity of each target comparing 1% DMSO to control, no DMSO was also determined. As shown in FIG. 16, knockdown of ISYNA1, led to higher AAV titers. Knockdown of GJB3 which was upregulated in DMSO treated cells showed reduced AAV production.

### Example 7. Reconstruction of the AAV Biosynthesis Pathway-Network-Based Analysis

To gain a more detailed understanding of the gene expression changes within the AAV biosynthesis pathway, the reconstruction and integrated transcriptomic data was visualized to perform network-based analyses. In this network, each node represents a gene and edges are established between nodes that share the same process, subsystem, or system. Edge weights were computed as a weighted sum of shared annotations, with higher contributions from shared processes, followed by subsystems, and then systems. Node positioning was determined using the Fruchteman-Reingold force-directed algorithm, resulting in a spatial organization that shows a separation of nodes according to system- and subsystem-level annotations.

Differential gene expression data was then incorporated into this network at each timepoint and for each regulation direction (up- and down-regulated genes), allowing the visualization of transcriptional changes in the context of network topology. The log 2FoldChange values were used to adjust edge weights based on log 2FoldChange similarity between two nodes. The edge weights were multiplied by the similarity of Log 2FoldChange values between connected nodes. Using the network with modified edges, initially, the Louvain method for community detection was applied. To ensure robustness and reproducible clustering, consensus clustering was combined with the Louvain clustering method. The community structures were evaluated across a range of resolution parameters and the optimal resolution was selected based on modularity and normalized mutual information (NMI) relative to known system-level annotations.

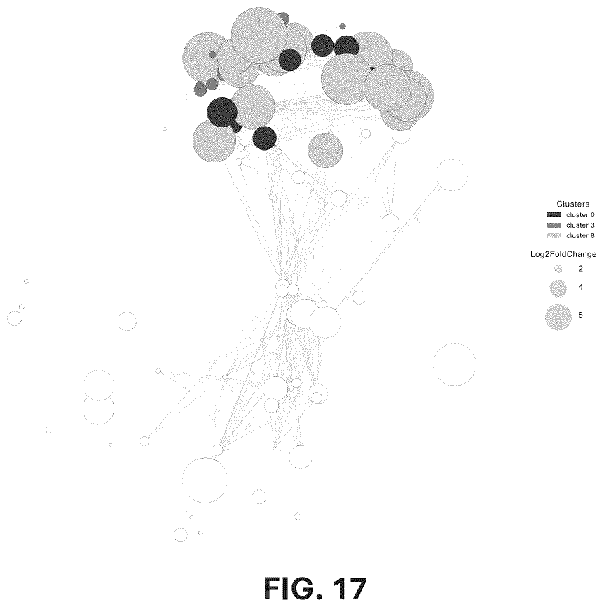

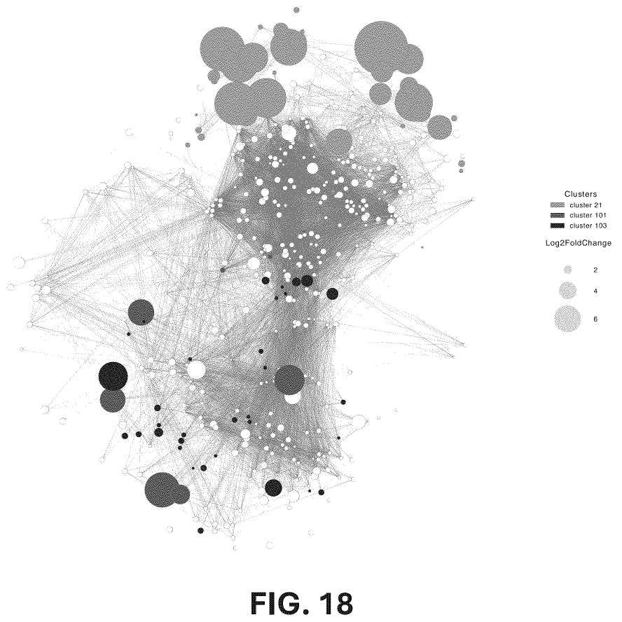

The resulting clusters represent groups of genes that shared both similar functional roles, annotated based on the AAV reconstruction, and similar expression changes across different media conditions. Distinct clusters were observed at early time points (0, 3, and 6 hpi) among up-regulated genes in production media compared to growth media (see FIG. 17 for the 0 hpi and FIG. 18 for the 3 hpi clusters). Down-regulated genes or later time points displayed weaker clustering and smaller log 2FoldChange magnitudes, suggesting that formation of these clusters may have been primarily driven by topological proximity in the reference network rather than the differential expression. These observations suggest that the transcriptional responses to media conditions are most pronounced during the initial stages of AAV biosynthesis.

To functionally characterize the resulting clusters, enrichment analysis was performed using Fisher's exact test to identify significantly overrepresented subsystems and processes. At the early timepoints, clusters with stronger internal connectivity and larger log 2FoldChange magnitudes were enriched for ‘Ad5 binding’ which is a subsystem representing the initial stage of Ad5 infection of producer cells required to initiate AAV biomanufacturing. Majority of the genes within these clusters were also annotated under the process ‘lysosomal exocytosis,’ which is nested within the ‘Ad5 binding’ subsystem and is known to be indirectly involved in viral entry by facilitating lipid composition via ASMase. Additionally, clusters related to ‘DNA repair’ subsystem were identified. Collectively, these results demonstrate that our AAV reconstruction network provides a resource that enables systemic analysis of transcriptomic data in the context of AAV biosynthesis. By integrating expression dynamics with curated biological relationships, this approach enabled the identification of functionally relevant gene clusters, highlighting key time points of regulatory divergence, and offered mechanistic insights into AAV biosynthesis. The genes identified in this example are recited below in Table 7.

TABLE 7
Genes identified in network clustering analysis
at 0 hpi and 3 hpi that are upregulated

| condition | cluster | gene |
|---|---|---|
| 0h_up | 0 | ACTA1 |
| 0h_up | 0 | GCGR |
| 0h_up | 0 | KCNB1 |
| 0h_up | 0 | SYT1 |
| 0h_up | 0 | SYT5 |
| 0h_up | 0 | MAP1B |
| 0h_up | 0 | NPM2 |
| 0h_up | 3 | MYH11 |
| 0h_up | 3 | MYH3 |
| 0h_up | 3 | MYH7B |
| 0h_up | 3 | EXOC3L1 |
| 0h_up | 3 | SCRIB |
| 0h_up | 3 | LAT2 |
| 0h_up | 3 | SYT2 |
| 0h_up | 3 | BAIAP3 |
| 0h_up | 3 | SYTL1 |
| 0h_up | 3 | NR4A3 |
| 0h_up | 8 | ITGAM |
| 0h_up | 8 | MYH15 |
| 0h_up | 8 | ACTC1 |
| 0h_up | 8 | HAP1 |
| 0h_up | 8 | RAB25 |
| 0h_up | 8 | BTK |
| 0h_up | 8 | NKD2 |
| 0h_up | 8 | TRIM72 |
| 0h_up | 8 | NLRP5 |
| 0h_up | 8 | RAB44 |
| 0h_up | 8 | H4C7 |
| 0h_up | 8 | ATP1A4 |
| 0h_up | 8 | ATP1B2 |
| 0h_up | 8 | ATP2B2 |
| 0h_up | 8 | GHSR |
| 0h_up | 8 | PTGER3 |
| 0h_up | 8 | HCAR1 |
| 0h_up | 8 | ADCY4 |
| 3h_up | 21 | SNRNP70 |
| 3h_up | 21 | RIF1 |
| 3h_up | 21 | CXADR |
| 3h_up | 21 | GSK3B |
| 3h_up | 21 | ITGAM |
| 3h_up | 21 | MYH11 |
| 3h_up | 21 | MYH15 |
| 3h_up | 21 | ACTA1 |
| 3h_up | 21 | ACTC1 |
| 3h_up | 21 | EXOC3L1 |
| 3h_up | 21 | NAPA |
| 3h_up | 21 | LIN7C |
| 3h_up | 21 | STX3 |
| 3h_up | 21 | HAP1 |
| 3h_up | 21 | LAT2 |
| 3h_up | 21 | RAB40C |
| 3h_up | 21 | BTK |
| 3h_up | 21 | GCGR |
| 3h_up | 21 | TXLNA |
| 3h_up | 21 | NKD2 |
| 3h_up | 21 | VPS18 |
| 3h_up | 21 | CBL |
| 3h_up | 21 | TRIM72 |
| 3h_up | 21 | SYT2 |
| 3h_up | 21 | NR4A3 |
| 3h_up | 21 | CLTC |
| 3h_up | 21 | WASL |
| 3h_up | 21 | SYNJ2 |
| 3h_up | 21 | GAK |
| 3h_up | 21 | DYNLRB1 |
| 3h_up | 21 | DCTN5 |
| 3h_up | 21 | TNPO2 |
| 3h_up | 21 | ADORA2A |
| 3h_up | 21 | ATP1B2 |
| 3h_up | 21 | CRHR2 |
| 3h_up | 21 | GHSR |
| 3h_up | 21 | NFKBIA |
| 3h_up | 21 | VAV3 |
| 3h_up | 21 | ADCY10 |
| 3h_up | 21 | NPM2 |
| 3h_up | 101 | XRN2 |
| 3h_up | 101 | CCNA1 |
| 3h_up | 101 | BAZ1B |
| 3h_up | 101 | PIAS1 |
| 3h_up | 101 | UNC5A |
| 3h_up | 101 | DAPK1 |
| 3h_up | 101 | TNFSF10 |
| 3h_up | 101 | CD14 |
| 3h_up | 103 | YY1 |
| 3h_up | 103 | POLE3 |
| 3h_up | 103 | POLE |
| 3h_up | 103 | POLD1 |
| 3h_up | 103 | FEN1 |
| 3h_up | 103 | RPA4 |
| 3h_up | 103 | H2BC5 |
| 3h_up | 103 | H4C8 |
| 3h_up | 103 | TERF2 |
| 3h_up | 103 | RNF168 |
| 3h_up | 103 | RAD9A |
| 3h_up | 103 | TOP3A |
| 3h_up | 103 | PIAS4 |
| 3h_up | 103 | TRIM25 |
| 3h_up | 103 | UBA7 |
| 3h_up | 103 | FANCA |
| 3h_up | 103 | SPHK1 |
| 3h_up | 103 | POLH |
| 3h_up | 103 | REV1 |
| 3h_up | 103 | ALKBH5 |
| 3h_up | 103 | RNF111 |
| 3h_up | 103 | NFRKB |
| 3h_up | 103 | INO80 |
| 3h_up | 103 | COPS3 |
| 3h_up | 103 | EME2 |
| 3h_up | 103 | POLN |
| 3h_up | 103 | PAXIP1 |
| 3h_up | 103 | RNF4 |
| 3h_up | 103 | PPP4R2 |
| 3h_up | 103 | RTEL1 |
| 3h_up | 103 | EYA3 |
| 3h_up | 103 | EYA4 |
| 3h_up | 103 | MBD4 |

### Example 8. Decreasing Levels of GAPDH Results in Higher AAV Titer Produced from HEK293 Cells

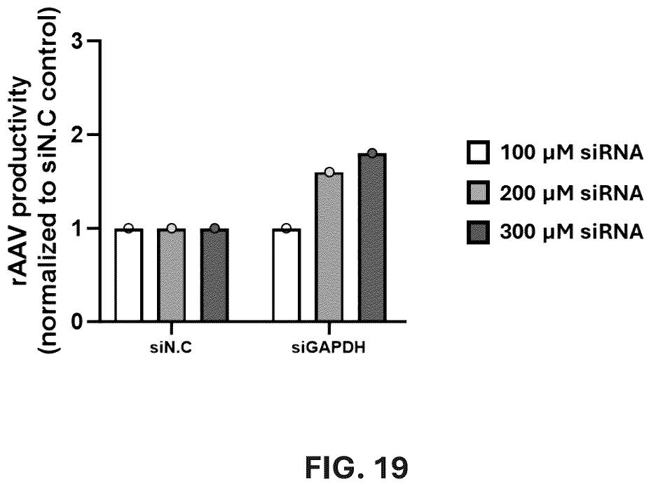

HEK293 cells were transfected with siRNA to GAPDH (siGAPDH) to decrease GAPDH expression or with a control siRNA (siN.C) along with AAV production transfection constructs pAdhelper and a triple-play construct consisting of AAV Rep, Cap, and ITR-transgene. The transfected cells were cultured for three days before harvesting to assess AAV titer. AAV titer was assessed by real-time PCR (qPCR). Results from siGAPDH were normalized to the siN.C control (FIG. 19) Three different concentrations of siRNA in the culture media were tested: 100 μM, 200 μM, and 300 μM per milliliter of HEK293 cell culture. rAAV production increased with GAPDH knockdown and showed trend of siGAPDH dose dependent increase.

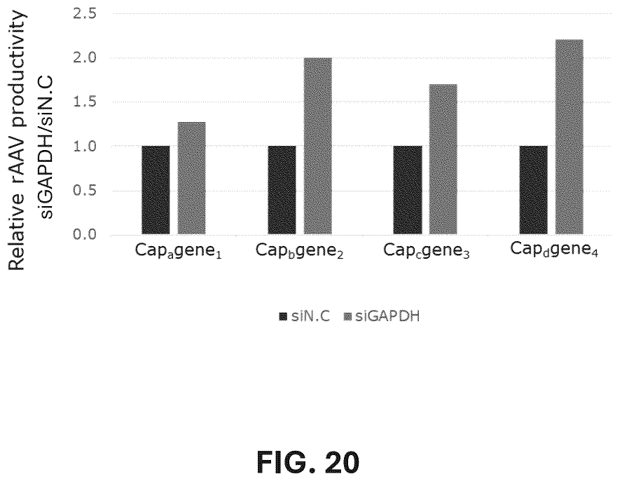

HEK293 cells were then transfected with 100 μM of either siGAPDH or siN.C along with the AAV production transfection constructs pAdhleper and a triple-play construct. Four different AAV constructs comprising different transgenes (Capₐgene1, Cap_{b}gene₂, Cap_{c}gene₃, and Cap_{d}gene₄) were used to assess rAAV productivity from HEK293 cells. Virus production was increased with all four constructs when GAPDH expression was decreased (FIG. 20).

These results demonstrate increased AAV production with a decrease in GAPDH expression in producer cells (e.g., HEK293 cells).

### Example 9. Growth Factor Receptors & RTK-Associated Proteins Modulate AAV Titer Production

### EGFR (Epidermal Growth Factor Receptor)

EGFR is a receptor tyrosine kinase that, upon ligand binding and dimerization, activates multiple cascades (RAS/RAF/MEK/ERK MAPK pathway, PI3K/Akt, JAK/STAT, PLCγ) promoting proliferation, survival, migration, and inflammatory responses. Noh, S. S. & Shin, H. J. Role of virus-induced EGFR trafficking in proviral functions. Biomolecules 13, 1766 (2023). In adenovirus infection, EGFR plays a multifaceted role. The physical process of adenovirus entry (binding to its primary receptor and integrins) can transiently activate EGFR or EGFR-related signaling as a stress response, even without EGF ligand. Id. This “stress-induced” EGFR activation helps trigger the host innate immune response. For example, adenovirus entry rapidly stimulates the Raf/MEK/ERK pathway and immediate early cytokines like IL-8 in epithelial cells. Bruder, J. T. & Kovesdi, I. Adenovirus infection stimulates the Raf/MAPK signaling pathway and induces interleukin-8 expression. J. Virol. 71, 398-404 (1997). However, adenovirus also downregulates EGFR during infection: the adenoviral E3 region proteins (e.g. RIDα/β complex) bind and reroute EGFR to endosomes/lysosomes, causing EGFR degradation. Noh et el. (2023), Biomolecules 13, 1766. This occurs within the early phase and serves to limit NF-κB signaling and inflammatory/apoptotic responses that EGFR might mediate. Id. Additionally, adenovirus E1A and E4orf1 oncoproteins manipulate EGFR pathways: E1A can repress some growth genes, and E4orf1 hijacks EGFR's tyrosine kinase activity in a ligand-independent manner to activate Ras/ERK and PI3K signaling. Kong, K., Kumar, M., Taruishi, M. & Javier, R. T. Adenovirus E4-ORF1 dysregulates epidermal growth factor and insulin/insulin-like growth factor receptors to mediate constitutive Myc expression. J. Virol. 89, 10774-10785 (2015). E4orf1 forms a complex with EGFR (and the membrane scaffold Dlg1) and even links EGFR with insulin/IGF receptors, resulting in constitutive EGFR phosphorylation and downstream signaling. Id. Biological context: Early on, EGFR activation contributes to pro-viral conditions (enhancing cell survival and suppressing antiviral cytokines) but must be balanced; the virus later dampens EGFR to avoid excessive inflammation. Noh et el. (2023), Biomolecules 13, 1766. In HeLa cells, which natively express EGFR, wild-type adenovirus infection would transiently exploit EGFR signaling to promote an environment favorable for viral DNA replication and to suppress interferon/NF-κB responses, then reduce surface EGFR to prevent the cell from undergoing inflammation-induced apoptosis.

Differential expression analysis revealed that EGFR expression was largely stable across cell lines and media conditions, with no significant differences between PCL1 and PCL2 at any time point barring 0 hpi on control media (Log 2FC=−0.43; adjusted p-value=5E-02). Temporal analysis indicated that on control media, EGFR levels were higher at 0 hpi than at 24 hpi in both PCLs, consistent with a transient reduction following infection, with this change being significant in PCL1 (Log 2FC=−0.6; adjusted p=9E10-04). This early suppression aligns with the known adenoviral strategy of dampening EGFR signaling to limit host inflammatory responses. In contrast, PCL2 on supplemented media exhibited a modest rebound in EGFR expression by 48 hpi (Log₂FC=−0.6; adjusted p=1E-02), suggesting that under nutrient-enriched conditions EGFR activity may be partially restored or sustained during the productive phase. Consistent with this, receptor subset enrichment analyses showed that DMSO supplementation of PCL2 enriched for receptors, including EGFR, within the PI3K-Akt signaling pathway at 0 hpi relative to 24 hpi (fold enrichment=1.6; adjusted p-value=4E-02), and within the PI3K-Akt (fold enrichment=3.1; adjusted p-value=5E-03), and MAPK (fold enrichment=2.0; adjusted p-value=2E-02) signaling pathways at 24 hpi relative to 48 hpi, reflecting a temporal re-engagement of growth and survival signaling as infection progresses. Together, these observations support the rationale for EGFR overexpression in PCL1 on control media prior to 48 hpi, as this condition lacks the late-phase recovery seen in PCL2. Enhancing EGFR signaling may therefore offset viral downregulation, sustaining proliferative and anti-apoptotic cascades via RAS/RAF/MEK/ERK, PI3K/Akt, and JAK/STAT pathways that favor continued AAV production.

### IGF1R (Insulin-Like Growth Factor 1 Receptor) & INSR (Insulin Receptor)

IGF1R and INSR are homologous receptor tyrosine kinases that trigger strong PI3K/Akt and MAPK signaling in response to IGF or insulin, generally promoting metabolism, protein synthesis, cell survival, and proliferation. HeLa cells in culture often have IGF1R and can respond to growth factors in serum. Adenovirus E4orf1 protein specifically exploits the InsR/IGF1R axis: E4orf1 binds the cell scaffold protein Dlg1 and forms a complex that includes EGFR and InsR/IGF1R at the plasma membrane. This leads to constitutive activation of PI3K and maintenance of high Akt activity, as well as ligand-independent signaling through Ras/ERK via EGFR. Kong et al. (2015), J. Virol. 89, 10774-10785. One outcome is sustained Myc protein levels in the nucleus, which adenovirus uses to drive transcription of genes advantageous for viral replication. Id. InsR/IGF1R normally can negatively crosstalk with EGFR (they can moderate EGFR activity), but E4orf1 by binding Dlg1 appears to antagonize that restraint, allowing maximal EGFR-Ras signaling. Id. Beyond E4orf1, the strong Akt signaling from IGF1R/InsR can prevent premature apoptosis of the host cell, giving the virus more time to replicate. In infection context: While adenovirus doesn't bind IGF1R directly, it co-opts downstream survival signaling. In a helper-virus scenario for AAV, the presence of adenovirus might activate PI3K/Akt via E4orf1 and serum growth factors, which could enhance cell survival and productivity of viral vectors. On the flip side, if insulin/IGF signaling is blocked, cells may undergo apoptosis faster in response to infection. Thus, IGF1R/InsR pathways are part of the pro-survival program induced by adenovirus early during infection.

Differential expression analysis showed that IGF1R expression remained largely unchanged across cell lines under both control and supplemented media, reflecting stable baseline expression. However, expression was significantly higher in control media relative to supplemented media for PCL1 at 0 (Log 2FC=−0.5; adjusted p-value=1E-02), 24 (Log 2FC=−0.6; adjusted p-value=1E-02), and 48 hpi (Log 2FC=−0.6; adjusted p-value=8E-03), and for PCL2 at 0 hpi (Log 2FC=−0.4; adjusted p-value=2E-02), indicating that supplementation broadly suppresses IGF1R transcription in PCL1 throughout infection and transiently in PCL2 during early infection. Temporal comparisons further revealed that on both control and supplemented media for either PCL, IGF1R levels were higher at 0 hpi than at 24 hpi (−1.3<Log 2FCs<−1.5; adjusted p-values<0.05), consistent with an early infection-associated reduction in receptor expression. Given that IGF1R activity promotes PI3K/Akt and MAPK signaling to support metabolism and survival, this pattern suggests a general downregulation of growth factor signaling following infection, with supplementation modulating the extent of this suppression. Accordingly, attenuation of IGF1R in PCL1 prior to 24 hpi under supplemented conditions may help rebalance this axis, reducing redundant Akt signaling and preventing excessive survival activity that competes with transcriptional reprogramming and viral production. This interpretation aligns with the known role of adenoviral E4orf1 in hijacking the InsR/IGF1R complex to sustain constitutive Akt activation and Myc stabilization, underscoring how partial dampening of IGF1R could relieve viral or metabolic overactivation and enhance process efficiency. Pathway enrichment provided additional context for this transcriptional behavior: on control media, PCL1 was enriched for receptor subsets, including IGF1R, participating in the FoxO (fold enrichment=3.74; adjusted p-value=1E-03), mTOR (fold enrichment=3.32; adjusted p-value=9E-04), and HIF-1 (fold enrichment=3.42; adjusted p-value=3E-03) signaling pathways at 0 hpi relative to 24 hpi, while PCL2 showed enrichment for receptor subsets within PI3K-Akt (fold enrichment=1.6; adjusted p-value=4E-02), HIF-1 (fold enrichment=2.75; adjusted p-value=4E-02), and FoxO (fold enrichment=4.00; adjusted p-value=1E-03) pathways under the same comparison. On supplemented media, PCL1 retained enrichment for mTOR signaling (fold enrichment=3.70; adjusted p-value=3E-06), whereas PCL2 was enriched for PI3K-Akt (fold enrichment=1.55; adjusted p-value=4E-02) and mTOR (fold enrichment=3.10; adjusted p-value=6E-4) pathways at 0 hpi relative to 24 hpi, suggesting that both cell lines transiently engage growth and metabolic signaling programs centered around IGF1R during early infection, though supplementation modifies their amplitude and composition. Given that IGF1R activity drives PI3K/Akt and MAPK cascades to support metabolism and survival, these enrichment trends imply a coordinated downregulation of growth factor-linked signaling following infection, with supplementation tempering but not eliminating this early signaling pulse. Accordingly, attenuation of IGF1R in PCL1 prior to 24 hpi under supplemented conditions could help rebalance this axis, reducing redundant Akt activity and preventing excessive survival signaling that competes with transcriptional reprogramming and viral production, underscoring how partial dampening of IGF1R could relieve viral or metabolic overactivation and enhance process efficiency.

### GRB2 (Growth Factor Receptor-Bound Protein 2)

GRB2 is an adaptor protein (not a receptor) that links activated receptors to the RAS/MAPK pathway. It contains SH2/SH3 domains and typically binds phospho-tyrosines on receptors like EGFR or on focal adhesion kinase, then recruits the SOS guanine nucleotide exchange factor to activate RAS. Bruder et al. (1997), J. Virol. 71, 398-404. In adenovirus infection of HeLa, GRB2 is rapidly engaged as part of the integrin-mediated entry signaling. Adenovirus uses penton base proteins with an RGD motif to bind cellular integrins, which activates focal adhesion kinase (FAK) and other kinases. This leads to recruitment of GRB2-SOS and activation of Ras and Raf. Indeed, within ˜20 minutes of adenoviral attachment, the Raf/MAPK/ERK cascade is turned on, leading to transcription of IL-8 and other early response genes². This occurs even in the presence of cycloheximide (no new proteins made), indicating it's due to pre-existing signaling components like GRB2. Id. The functional outcome is an inflammatory signal (IL-8) that can recruit immune cells, but it may also condition the cell for viral replication. Adenovirus benefits from moderate MAPK activation: it can induce pro-survival genes via NF-κB/AP-1, yet if excessive, inflammation could harm the cell. The virus later uses E3 proteins to tone down these signals. Summary: GRB2 is essential for linking adenovirus entry to intracellular signaling. In HeLa cells, integrin engagement by adenovirus likely triggers GRB2-dependent Ras/MAPK activation, contributing to the early innate response and potentially aiding viral genome transit to the nucleus. Id. By the time AAV is being produced (with adenovirus help), these GRB2-mediated signals might influence cellular stress responses that affect viral vector yields.

Differential expression analysis indicated that GRB2 expression was stable across cell lines and media types, showing no significant differences between PCLs under baseline conditions. Temporal comparisons, however, revealed consistently higher GRB2 expression at 0 hpi relative to 24 hpi on both control and supplemented media for PCL1 and PCL2 (−0.83<Log 2FC<−0.68; p-adjusted values<0.05), as well as greater expression at 24 hpi relative to 48 hpi in both cell lines under each media condition (−0.62<Log 2FC<−0.25; p-adjusted values<0.05), indicating a progressive decline in GRB2 transcript abundance over the course of infection. Pathway enrichment analysis contextualized this temporal trend, showing that at 0 hpi relative to 24 hpi, PCL1 was enriched for receptor subsets including GRB2 that participate in FoxO (fold enrichment=3.74; p-adjusted value=1E-03) and mTOR (fold enrichment=3.32; p-adjusted value=9E-04) signaling on control media and in mTOR (fold enrichment=3.70; p-adjusted value=3E-06) signaling on supplemented media, while PCL2 was enriched for receptor subsets linked to FoxO (fold enrichment=4.00; p-adjusted value=1E-03), PI3K-Akt (fold enrichment=1.57; p-adjusted value=4E-02), and mTOR (fold enrichment=3.56; p-adjusted value=4E-04) signaling under control conditions, and PI3K-Akt (fold enrichment=2.04; p-adjusted value=4E-02) and mTOR (fold enrichment=3.06; p-adjusted value=6E-04) pathways under supplementation. Since GRB2 acts as a key adaptor coupling receptor activation to Ras/MAPK and PI3K/Akt signaling, its early enrichment within these pathways indicates strong integration of growth factor and metabolic inputs during infection onset. The subsequent decline in GRB2 expression, coinciding with Ad5 E3 and E4-mediated suppression of MAPK and NF-κB activity, likely reflects a controlled viral mechanism to reduce host stress and inflammatory signaling. In this context, attenuating GRB2 expression in PCL1 under supplemented conditions prior to 24 hpi could mimic this natural downregulation, tempering MAPK-dependent cytokine and stress responses while preserving the beneficial early Ras- and Akt-mediated survival and metabolic activation that support productive AAV biosynthesis.

### Methods of Example 9

### Library Preparation

The RNA sequencing libraries were prepared using the NEBNext Ultra II RNA Library Prep Kit for Illumina using manufacturer's instructions (New England Biolabs, Ipswich, MA, USA). Briefly, mRNAs were initially enriched with Oligod(T) beads. Enriched mRNAs were fragmented for 15 minutes at 94° C. First strand and second strand cDNA were subsequently synthesized. cDNA fragments were end repaired and adenylated at 3′ends, and universal adapters were ligated to cDNA fragments, followed by index addition and library enrichment by PCR with limited cycles. The sequencing library was validated on the Agilent TapeStation (Agilent Technologies, Palo Alto, CA, USA), and quantified by using Qubit 2.0 Fluorometer (ThermoFisher Scientific, Waltham, MA, USA) as well as by quantitative PCR (KAPA Biosystems, Wilmington, MA, USA).

### Sequencing

The sequencing libraries were multiplexed and clustered onto an Illumina flowcell. After clustering, the flow cell was loaded onto the Illumina NovaSeq 6000 instrument according to the manufacturer's instructions. The samples were sequenced using a 2×150 bp Paired End (PE) configuration and targeting ˜30 million reads/sample. Image analysis and base calling were conducted by the Illumina Control Software. Raw sequence data (.bcl files) generated from Illumina was converted into fastq files and de-multiplexed using Illumina bcl2fastq 2.20 software. One mis-match was allowed for index sequence identification.

The human comprehensive gene annotation and reference genome for GRCh38.p13 release 38 primary assembly was retrieved from Gencode in GTF and fasta format respectively. Ad5 gene annotation and reference genome was retrieved from NCBI (RefSeq accession AC_000008.1; GenInfo accession 56160529; RefSeq assembly GCA_000857865.1; GenBank assembly GCF_000857865.1; Name ViralProj15107). AAV gene annotation and reference genome was provided by Sanofi. Both genome indexing and alignment of trimmed reads were performed using the STAR aligner (v2.7.3a). Prior to alignment, quality control of paired-end fastq reads through sequential application of FastQC (v0.11.9), Trimmomatic (v0.39) and FastQC on trimmed reads ensured removal of low-quality and adapter sequences. Following alignment, both raw and transcript-per-million (TPM) read count quantification from the binary alignment map (BAM) file was performed using HTSeq (v0.11.3), SAMtools (v1.10) and StringTie (version 2.1.1). Alignment, QC, and readcount quantification was performed using a nextflow (v #) pipeline as hosted on a docker instance of Ubuntu 18.04.

### DESeq2

Differential gene expression (DE) analysis was performed via DESeq2 (v1.38.3) using the R (v4.2.2) programming language. Love, M. I., Huber, W. & Anders, S. Moderated estimation of fold change and dispersion for RNA-seq data with DESeq2. Genome Biol. 15, 550 (2014). Prior to testing, all genes with 0 recorded counts across samples are removed from the analysis. Using the raw count matrix and Ensembl annotations (version 104) output from the rnaseq_docker_nextflow pipeline (commit 179f36b) as input to the testing framework, and following normalization by relative log expression (RLE) scaling factors, DE testing was performed between choice of (i) media and (ii) cell line at each timepoint post AAV transfection, and (iii) between each timepoint post AAV transfection in a given media and cell line. The resulting 24 sets of DEGs (Bonferroni adjusted p value<0.05) were subsequently interrogated in ligand receptor enrichment analyses.

### Gene Background

To ensure biological relevance of results in the context of each DESeq2 comparison, a gene background (i.e., all expressed genes) was curated consisting of all non-DE genes with baseMean expression within or greater than the baseMean expression range of each DESeq2 comparison.

### Manual Curation of the Ad5 Immune Response Network

The Ad5 immune response network was constructed by integrating host signaling and viral targeting information to contextualize the cellular processes modulated during adenovirus type 5 (Ad5) infection. A total of 45 signal transduction, cell growth and death, and immune system networks were downloaded from the KEGG database and iteratively merged to form a unified host signaling framework encompassing innate immune, stress-response, apoptotic, and metabolic regulation pathways. Kanehisa, M., Furumichi, M., Tanabe, M., Sato, Y. & Morishima, K. KEGG: new perspectives on genomes, pathways, diseases and drugs. Nucleic Acids Res. 45, D353-D361 (2017). Within this framework, known adenoviral targets were curated through literature review, focusing on experimentally validated interactions between Ad5-encoded proteins (E1B, E3, and E4) and host signaling components. These curated viral-host interactions were overlaid onto the merged KEGG signaling network to generate an Ad5-specific immune response network capturing the breadth of host pathways directly or indirectly targeted by viral proteins. Visualization and manual refinement were performed in Cytoscape (v3.9.1). Shannon, P. et al. Cytoscape: a software environment for integrated models of biomolecular interaction networks. Genome Res. 13, 2498-2504 (2003). Individual pathway maps were extracted from the global network as necessary to highlight specific receptor-linked signaling routes or viral interference points referenced in the main text.

### Ligand Receptor Analysis

Following the application of the DESEQ2 analysis pipeline DEG results were subset for genes annotated as a participating ligand or receptor of an LR pair as annotated in CellChat (Jin, S. et al. Inference and analysis of cell-cell communication using CellChat. Nat. Commun. 12, 1088 (2021)), NeuronChat (Zhao, W., Johnston, K., Ren, H., Xu, X. & Nie, Q. Inferring neuron-neuron communications from single-cell transcriptomics through NeuronChat. Nature Communications 14, (2023)), Stadtmauer et al (Stadtmauer, D. J. et al. Cell type and cell signalling innovations underlying mammalian pregnancy. Nat. Ecol. Evol. 9, 1469-1486 (2025)) and Uniprot (UniProt Consortium. UniProt: the universal protein knowledgebase in 2021. Nucleic Acids Res. 49, D480-D489 (2021)). Subsequent filtering of all mapped LR pairs on the manually curated Ad5 immune response network through shared receptor EntrezIDs yielded a network-specific set of LR pairs. To account for receptors in the Ad5 immune response network that were not encompassed in this set for downstream enrichment analyses, the Ad5 immune response network was subset for uniprot identifiers associated with the uniprot keyword KW-0675 corresponding to receptor molecular function.

Corresponding enriched receptors of LR pairs and enriched pathways were found using the enrichr function of the clusterProfiler R package (v4.16.0) with default arguments. Yu, G., Wang, L.-G., Han, Y. & He, Q.-Y. clusterProfiler: an R package for comparing biological themes among gene clusters. OMICS 16, 284-287 (2012). For receptor enrichment analyses, a total of 3 ORA were run on the receptors of LR pairs for each comparison of PCL, media supplementation, and hpi corresponding to i) bi-directional ii) up- and iii) down-regulated DE genes. In all ORAs, background was set to receptors of the Ad5 immune response network, and the set of input DE receptors was filtered for a DE ligand in the corresponding LR pairs. Additionally, LR pairs with a complex ligand were required to exhibit consistent expression directionality across all components of the ligand complex. For pathway enrichment analyses, this process was repeated using non-receptor DE genes of the Ad5 immune response network as input and the same network filtered for receptors as background. In all ORAs, pathway selection was constrained to those constituting the Ad5 immune response network. ORA results were filtered for a nominal p-value<0.05, Benjamini-Hochberg adjusted p-value<0.05, and q-value<0.2. Adjusted p-values were computed using the Benjamini-Hochberg procedure, and q-values were calculated using Storey's method implemented in the qvalue R package (v2.40.0). Dabney, A. R. & Storey, J. D. Bioconductor's qvalue package. hist (hedenfalk) (2006).

82024800v1

1. An engineered cell line in which the expression or activity of at least one of the transcription factor genes listed in Tables 1-4, one of the transcription factor target genes listed in Tables 1-4, or one of ADM2, AURKA, BCL2, CCND1, CHD1, CREB3L3, RIGI/DDX58, ECSIT, EHHADH, ERBB2, FAS, FBXW7, FGFR3, FOXL2, GATA1, HDAC5, HOXA5, HSP90B1, IL6, ITGA2B, KAT5, LAS1L, LIF, NFKB1, NFKB2, NFKB1A, NFE2L2, NGEF, NME1, NR3C1, POLN, PPAR-A, PTEN, RELB, RPA1, RXRA, SMAD4, SS18, TCF7, TGM2, THEM6, TNFAIP3, TP63, TRPC1, DBH, ITGAM, HHEX, TXNIP, ASNS, PLEKH02, CDKN1A, C5orf64, COL20A1, GJB3, ISYNA1, PARD6G-AS1, ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, YY1, EGFR, IGF1R, INSR, and GRB2 is altered as compared to a control cell line.

2-4. (canceled)

5. The engineered cell line of claim 1, wherein the cell line is modified by a modulator that increases or decreases the expression and/or activity of at least one gene, and/or the cell line comprises a genetic modification, optionally wherein the modulator comprises
(a) a nuclease, optionally wherein the nuclease is selected from the group consisting of a zinc finger nuclease (ZFN), a meganuclease, a transcription activator-like effector nuclease (TALEN), and a clustered regularly interspaced short palindromic repeats (CRISPR) system;
(b) a double stranded RNA (dsRNA), a small interfering RNA (siRNA), a small hairpin RNA (shRNA), a microRNA (miRNA), or an antisense RNA oligonucleotide (ASO);
(c) a template for homologous arm mediated recombination;
(d) an inhibitor of at least one gene selected from the group consisting of: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, TNFAIP3, EGFR, IGF1R, INSR, and GRB2, optionally wherein the inhibitor comprises a synthetic small molecule inhibitor;
(e) an agonist of at least one gene selected from the group consisting of: ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1, optionally wherein the agonist comprises a synthetic small molecule agonist.

6-14. (canceled)

15. The engineered cell line of claim 1, wherein the cell line is a mammalian cell line or an insect cell line,
optionally wherein the mammalian cell line is a CHO cell line, Vero cell line, HeLa cell line, MDCK cell line, BHK cell line, A549 cell line, amniocyte cell line, or HEK293 cell line, or optionally wherein the insect cell line is an Sf9 cell line or a Hi5 cell line.

16-18. (canceled)

19. The engineered cell line of claim 1, wherein the cell line is a host cell line for the production of viral particles, and/or the cell line is a producer cell line, optionally wherein
the producer cell line further comprises stably integrated nucleic acid sequences encoding for AAV rep and/or cap, AAV ITR, and a gene of interest,
the producer cell line further comprises AAV helper function encoding sequences of an AAV helper virus, optionally wherein the AAV helper function encoding sequences comprise at least one of: E1a, E1b, E2A, L4, E4, VA RNA,
the AAV helper virus is an adenovirus, optionally wherein the adenovirus is Ad5 or Ad2, and/or
the titer of rAAV produced from the engineered cell line is at least about 1.5-fold higher compared to the titer of rAAV produced from a cell line in which the expression of the gene is not altered.

20-33. (canceled)

34. A method for increasing the production of rAAV particles, wherein the method comprises culturing a cell in the presence of a modulator that alters the expression or activity of at least one gene, wherein
(a) the expression of at least one of the genes DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, and TNFAIP3 is reduced as compared to a control cell line; and/or
(b) in which the expression of at least one of the genes ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1 is increased as compared to a control cell line, under conditions that allow for production and/or secretion of the recombinant viral particles,
optionally wherein the method increases recombinant viral titer by at least 1.5-fold compared to a method comprising a control cell,
optionally wherein the modulator comprises an inhibitor or agonist of a pathway regulated by any one of the genes listed in (a) or (b),
optionally wherein the modulator comprises a template for homologous arm-mediated recombination.

35. (canceled)

36. The method of claim 34, wherein the modulator comprises
(a) a synthetic small molecule inhibitor of at least one of: DBH, ECSIT, EHHADH, FAS, FGFR3, FOXL2, HDAC5, HOAX1, HSP90B1, ISYNA1, ITGAM, KAT5, NFKB1, NFKBIA, POLN, RELB, RIGI/DDX58, SS18, THEM6, TNFAIP3, EGFR, IGF1R, INSR, and GRB2; and/or
(b) a synthetic small molecule agonist of at least one gene selected from the group consisting of: ASNS, BCL2, CDKN1A, COL20A1, GATA1, GJB3, LAS1L, LIF, NFKB2, NGEF, NME1, NR3C1, RPA1, SMAD4, TRPC1, TXNIP, PARD6G-AS1, PLEKHO2, and ACTA1, ACTC1, ADCY10, ADCY4, ADORA2A, ALKBH5, ATP1A4, ATP1B2, ATP2B2, BAIAP3, BAZ1B, BTK, CBL, CCNA1, CD14, CLTC, COPS3, CRHR2, CXADR, DAPK1, DCTN5, DYNLRB1, EME2, EXOC3L1, EYA3, EYA4, FANCA, FEN1, GAK, GCGR, GHSR, GSK3B, H2BC5, H4C7, H4C8, HAP1, HCAR1, INO80, KCNB1, LAT2, LIN7C, MAP1B, MBD4, MYH11, MYH15, MYH3, MYH7B, NAPA, NKD2, NLRP5, NPM2, NR4A3, NFRKB, PAXIP1, PIAS1, PIAS4, POLE, POLE3, POLD1, POLH, PPP4R2, PTGER3, RAB25, RAB40C, RAB44, RAD9A, REV1, RIF1, RNF111, RNF168, RNF4, RPA4, RTEL1, SCRIB, SNRNP70, SPHK1, STX3, SYT1, SYT2, SYT5, SYTL1, SYNJ2, TERF2, TNFSF10, TNPO2, TOP3A, TRIM25, TRIM72, TXLNA, UBA7, UNC5A, VAV3, VPS18, WASL, XRN2, and YY1,
optionally wherein the modulator comprises DMSO, DMF, NMP, or dihydrolevoglucosenone (Cyrene), optionally wherein the concentration of DMSO is at least about 0.5%,
optionally wherein the modulator comprises an inhibitor or agonist of a pathway regulated by any one of the genes listed in (a) or (b),
optionally wherein the modulator comprises a template for homologous arm-mediated recombination.

37-44. (canceled)

45. The method of claim 31, wherein the cell line is a mammalian cell line or an insect cell line,
optionally wherein the mammalian cell line is a CHO cell line, Vero cell line, HeLa cell line, MDCK cell line, BHK cell line, A549 cell line, amniocyte cell line, or HEK293 cell line,
optionally wherein the insect cell line is an Sf9 cell line or a Hi5 cell line, and/or
optionally wherein the rAAV comprises an AAV serotype selected from the group consisting of: AAV1, AAV2, AAV3, AAV4, AAV5, AAV6, AAV7, AAV8, AAVrh8, AAVrh8R, AAV9, AAV10, AAVrh10, AAV11, AAV12, AAV13, AAV DJ, a goat AAV, a bovine AAV, or a mouse AAV, AAV2/207m8, or AAV LK03, or hybrids, or variants or chimeras thereof.

46-57. (canceled)

58. A recombinant viral particle obtained from the engineered host cell line of claim 1,
optionally wherein the recombinant viral particle is a recombinant adeno-associated virus (rAAV) particle.

59. (canceled)

60. A method for producing a recombinant adeno-associated virus (rAAV) particle, the method comprising: transiently transfecting the host cell line or producer cell line of claim 19 with: (i) nucleic acids encoding AAV rep and/or AAV cap, (ii) nucleic acids encoding AAV ITRs, (iii) nucleic acid encoding a gene of interest,
optionally wherein one or more plasmids comprise the nucleic acids,
optionally further comprising transfecting the host cell line with a plasmid comprising AAV helper function encoding sequences of an AAV helper virus,
optionally wherein the AAV helper virus is an adenovirus, optionally wherein the adenovirus is Ad5 or Ad2,
optionally wherein the rAAV comprises an AAV serotype selected from the group consisting of: AAV1, AAV2, AAV3, AAV4, AAV5, AAV6, AAV7, AAV8, AAVrh8, AAVrh8R, AAV9, AAV10, AAVrh10, AAV11, AAV12, AAV13, AAV DJ, a goat AAV, a bovine AAV, or a mouse AAV, AAV2/207m8, or AAV LK03, or hybrids, or variants or chimeras thereof.

61-68. (canceled)

69. An engineered human embryonic kidney (HEK) 293 cell line in which the expression of glyceradehyde-3-phosphate dehydrogenase (GAPDH) is downregulated compared to a control HEK293 cell line, wherein the HEK293 cell line is a host HEK293 cell line for the production of viral particles,
optionally wherein the GAPDH is downregulated by a modulator that decreases the expression and/or activity of the GAPDH,
optionally wherein the cell line comprises a genetic modification, and/or
optionally wherein the modulator comprises:
(a) a nuclease, optionally wherein the nuclease is selected from the group consisting of a zinc finger nuclease (ZFN), a meganuclease, a transcription activator-like effector nuclease (TALEN), and a clustered regularly interspaced short palindromic repeats (CRISPR) system,
(b) a double stranded RNA (dsRNA), a small interfering RNA (siRNA), a small hairpin RNA (shRNA), a microRNA (miRNA), or an antisense RNA oligonucleotide (ASO),
(c) a template for homologous arm-mediated recombination, and/or
(d) an inhibitor of GAPDH, optionally wherein the inhibitor comprises a synthetic small molecule inhibitor.

70-77. (canceled)

78. The engineered cell line of claim 69, wherein the cell line is a producer cell line,
optionally wherein the producer cell line further comprises stably integrated nucleic acid sequences encoding for AAV rep and/or cap, AAV ITR, and a gene of interest,
optionally wherein the producer cell line further comprises AAV helper function encoding sequences of an AAV helper virus,
optionally wherein the AAV helper virus is an adenovirus, optionally wherein the adenovirus is Ad5 or Ad2,
optionally wherein the AAV helper function encoding sequences comprise at least one of: E1a, E1b, E2A, L4, E4, and VA, and/or
optionally wherein the titer of rAAV produced from the engineered cell line is at least about 1.5-fold higher compared to the titer of rAAV produced from a cell line in which the expression of the gene is not altered.

79-87. (canceled)

88. A method for increasing the production of rAAV particles, wherein the method comprises culturing a HEK293 cell in the presence of a modulator, wherein the expression or activity of glyceraldehyde-3-phosphate dehydrogenase (GAPDH) is decreased as compared to a control HEK293 cell line, under conditions that allow for production and/or secretion of the recombinant viral particles,
wherein the modulator optionally comprises:
(a) a synthetic small molecule inhibitor of GAPDH;
(b) an inhibitor of a pathway regulated by GAPDH;
(c) dimethylsulfoxide (DMSO), dimethylformamide (DMF), (N-methylpyrrolidone (NMP), dihydrolevoglucosenone (Cyrene), DC-5163, 1,2,3,4,6-penta-O-galloyl-β-D-glucopyranose (PGG), or 4-octyl itaconate (4-OI), optionally wherein the concentration of DMSO is at least about 0.5%;
(d) a nuclease, optionally wherein the nuclease is selected from the group consisting of a zinc finger nuclease (ZFN), a meganuclease, a transcription activator-like effector nuclease (TALEN), and a clustered regularly interspaced short palindromic repeats (CRISPR) system;
(e) a double stranded RNA (dsRNA), a small interfering RNA (siRNA), a small hairpin RNA (shRNA), a microRNA (miRNA), or an antisense RNA oligonucleotide (ASO); and/or
(f) a template for homologous arm-mediated recombination;
optionally wherein the method increases recombinant viral titer by at least 1.5-fold compared to a method comprising a control HEK293 cell.

89-97. (canceled)

98. An engineered HEK293 host cell line produced by a method comprising knocking down the expression or activity of glyceraldehyde-3-phosphate dehydrogenase (GAPDH), wherein the engineered HEK293 cell line is for the production of viral particles,
optionally wherein the knockdown is carried out using at least one of: homology-directed recombination, RNA-based knock-down, and a CRISPR-Cas enzymatic method,
optionally comprising stably integrated nucleic acid sequences encoding for AAV rep and/or cap, AAV ITR, and a gene of interest and optionally AAV helper function encoding sequences of an AAV helper virus,
optionally wherein the AAV helper virus is an adenovirus, optionally wherein the adenovirus is Ad5 or Ad2,
optionally wherein the AAV helper function encoding sequences comprise at least one of: E1a, E1b, E2A, L4, E4, and VA, and/or
optionally wherein the rAAV comprises an AAV serotype selected from the group consisting of: AAV1, AAV2, AAV3, AAV4, AAV5, AAV6, AAV7, AAV8, AAVrh8, AAVrh8R, AAV9, AAV10, AAVrh10, AAV11, AAV12, AAV13, AAV DJ, a goat AAV, a bovine AAV, or a mouse AAV, AAV2/207m8, or AAV LK03, or hybrids, or variants or chimeras thereof.

99-103. (canceled)

104. A recombinant viral particle obtained from the engineered HEK293 host cell line produced by the method according to claim 98,
optionally wherein the recombinant viral particle is a recombinant adeno-associated virus (rAAV) particle.

105. (canceled)

106. A method for producing a recombinant adeno-associated virus (rAAV) particle, the method comprising: transiently transfecting the host HEK293 cell line of claim 69 with one or more plasmids comprising: (i) nucleic acids encoding AAV rep and/or AAV cap, (ii) nucleic acids encoding AAV ITRs, and (iii) nucleic acid encoding a gene of interest,
optionally comprising transfecting the host HEK293 cell line with a plasmid comprising AAV helper function encoding sequences of an AAV helper virus,
optionally wherein the AAV helper virus is an adenovirus, optionally wherein the adenovirus is Ad5 or Ad2.

107-109. (canceled)

110. A method for producing a recombinant adeno-associated virus (rAAV) particle, the method comprising: stably transfecting the producer HEK293 cell line of claim 78 with: (i) nucleic acids encoding AAV rep and/or AAV cap, (ii) nucleic acids encoding AAV ITRs, and (iii) nucleic acid encoding a gene of interest,
optionally comprising transfecting the producer HEK293 cell line with a plasmid comprising AAV helper function encoding sequences of an AAV helper virus,
optionally wherein the AAV helper virus is an adenovirus, optionally wherein the adenovirus is Ad5 or Ad2.

111-113. (canceled)

114. The method according to claim 106, wherein the rAAV comprises an AAV serotype selected from the group consisting of: AAV1, AAV2, AAV3, AAV4, AAV5, AAV6, AAV7, AAV8, AAVrh8, AAVrh8R, AAV9, AAV10, AAVrh10, AAV11, AAV12, AAV13, AAV DJ, a goat AAV, a bovine AAV, or a mouse AAV, AAV2/207m8, or AAV LK03, or hybrids, or variants or chimeras thereof.


SEQUENCE LISTING (10 sequences)

SEQ ID NO: 1 - DNA, 23 bp, synthetic construct
  source 1..23: mol_type = other DNA
tctagttgccagccatctgttgt

SEQ ID NO: 2 - DNA, 18 bp, synthetic construct
  source 1..18: mol_type = other DNA
tgggagtggcaccttcca

SEQ ID NO: 3 - DNA, 21 bp, synthetic construct
  source 1..21: mol_type = other DNA
tcccccgtgccttccttgacc

SEQ ID NO: 4 - DNA, 21 bp, synthetic construct
  source 1..21: mol_type = other DNA
gtccttctaacacacctcctg

SEQ ID NO: 5 - DNA, 21 bp, synthetic construct
  source 1..21: mol_type = other DNA
caggctgtggaatgtatcgag

SEQ ID NO: 6 - DNA, 23 bp, synthetic construct
  source 1..23: mol_type = other DNA
tgtgccccattaaaccagttgcc

SEQ ID NO: 7 - DNA, 19 bp, synthetic construct
  source 1..19: mol_type = other DNA
ttgcgtcggtgttggagat

SEQ ID NO: 8 - DNA, 21 bp, synthetic construct
  source 1..21: mol_type = other DNA
caaggccaagatcgtgaagaa

SEQ ID NO: 9 - DNA, 20 bp, synthetic construct
  source 1..20: mol_type = other DNA
tgcaccacatttcggcccca

SEQ ID NO: 10 - protein, 4 aa, synthetic construct
YRPW